SEQUENCE LISTING (62 sequences)

SEQ ID NO: 1 - DNA, 1458 bp, synthetic construct
  source 1..1458: mol_type = other DNA
atggaactcttcttcatatgtggactagtactcttctccaccctatcactaatatccctcttcctcctccacaaccacagttctgctcgggggtacaggctgcccccgggcagaatgggatggcccttcataggcgagtcatacgagtttttagcaaacgggtggaaagggtacccggaaaagtttatatttagcaggttggccaagtataaaccgaatcaagtatttaagacgtcgatcctaggagaaaaagtcgcggtaatgtgtggcgcgacatgtaacaagttcttgttctcgaacgagggcaaattagtaaatgcttggtggccgaattcggttaataagatcttcccttcttctactcaaacttcttccaaggaagaagctaagaagatgcggaaacttctccctacattctttaaacccgaggcactacaacgatacatacccatcatggacgaaattgcgatccgacacatggaggacgaatgggaaggcaaatccaaaatcgaagtattcccactcgcaaaacgctacacattttggctagcgtgccgtctattcctaagcatagacgacccggtacacgtagccaaattcgctgacccgttcaacgacattgcctcagggatcatatcgatcccaatagacctccccggcacaccattcaaccggggaattaaggcctcgaatgtcgtgagacaggaattgaagaccataataaagcagaggaaattggacctgtccgacaacaaggcgtccccgacacaggatatattgtcacacatgttattaactcccgacgaagacgggcggtatatgaatgaattggacattgctgataaaattctcgggttgttaattggaggacatgatactgcaagtgctgcttgtacttttgttgtgaagtttcttgctgaactccctcatatttacgacggtgtttacaaagagcaaatggagatagcaaagtcgaaaaaagaaggagagcgattaaattgggaggacatacaaaagatgaaatattcatggaatgtggcctgtgaagtcatgcgtttagcacctcctcttcaaggcgcttttcgtgaagccctctctgattttatgtacgccggtttccaaattcccaagggttggaagttatattggagcgcaaactcaacacataggaacccagaatgcttcccagagccggaaaaattcgacccagcaaggttcgatgggagcggtccggccccatacacgtacgtaccgttcggaggagggccgagaatgtgcccaggaaaagagtatgcaaggctagaaatattggtgttcatgcacaacattgtcaagagatttaagtgggaaaaacttattcctgatgaaaccattgttgttaatcccatgccgaccccggctaaaggcctacccgtccgccttcgtcctcattccaaacccgtaactgtatctgcttaa

SEQ ID NO: 2 - protein, 485 aa, synthetic construct
MELFFICGLVLFSTLSLISLFLLHNHSSARGYRLPPGRMGWPFIGESYEFLANGWKGYPEKFIFSRLAKYKPNQVFKTSILGEKVAVMCGATCNKFLFSNEGKLVNAWWPNSVNKIFPSSTQTSSKEEAKKMRKLLPTFFKPEALQRYIPIMDEIAIRHMEDEWEGKSKIEVFPLAKRYTFWLACRLFLSIDDPVHVAKFADPFNDIASGIISIPIDLPGTPFNRGIKASNVVRQELKTIIKQRKLDLSDNKASPTQDILSHMLLTPDEDGRYMNELDIADKILGLLIGGHDTASAACTFVVKFLAELPHIYDGVYKEQMEIAKSKKEGERLNWEDIQKMKYSWNVACEVMRLAPPLQGAFREALSDFMYAGFQIPKGWKLYWSANSTHRNPECFPEPEKFDPARFDGSGPAPYTYVPFGGGPRMCPGKEYARLEILVFMHNIVKRFKWEKLIPDETIVVNPMPTPAKGLPVRLRPHSKPVTVSA

SEQ ID NO: 3 - DNA, 1509 bp, synthetic construct
  source 1..1509: mol_type = other DNA
atggagctaattaccttactaagtgctcttcttgttcttgctatagtgagtttatctacatttttcgtcctttactataatactcctactaaggacggcaaaactctccctcccggtcgtatgggctggccttttataggcgagtcctacgacttttttgccgccggttggaaagggaagcccgagagcttcattttcgaccggttgaagaaatttgctaaggggaacctgaacggtcagttcaggacgagcttgtttgggaacaagtcgattgtggtggcgggggctgctgctaacaagcttcttttctcgaatgaaaagaagcttgttaccatgtggtggcccccgtctattgataaggccttcccgtcgactgcacagttgagtgcgaacgaggaggccttattgatgaggaagttttttccttcttttttgattagaagggaggcgctccagcgctacatccctattatggacgactgcacccgtcgtcacttcgcgacgggtgcgtggggtccgtcggacaagatcgaggccttcaatgtgacccaagactacacgttttgggtcgcctgcagagtcttcatgagcatagacgctcaggaagaccctgagacggtagactccctctttaggcactttaacgtgcttaaagcgggaatctactcaatgcacatcgatctcccgtggacgaacttccaccacgcgatgaaggcgtcccacgccatcaggagcgccgtggagcaaatcgcgaagaaaagaagggcggaattggccgagggaaaggcgttcccgacacaagatatgctgtcttacatgctcgaaacgccaattacatcggcggaggatagcaaggacgggaaagcgaagtatttgaatgacgccgatatcgggacgaagatacttggtcttcttgttggtggccatgacacaagtagtacagttattgccttctttttcaagttcatggctgaaaatcctcatgtttatgaggctatttacaaagaacaaatggaggtagcggccacaaaagcgccgggggagcttctaaattgggatgacttgcagaaaatgaagtactcgtggtgtgcgatttgcgaggttatgcgtttgactccccctgtccaaggcgcctttcgccaagccatcaccgacttcacccataatggttaccttattcccaagggttggaagatatactggagtacacactcaacacacagaaatcccgaaatcttcccacaaccagagaaattcgacccaacaagattcgaaggaaacgggccaccagcgttctcattcgtgccattcggaggaggcccgagaatgtgtccgggtaaagaatatgcaaggctacaagtgcttacatttgtgcaccacattgtgaccaaattcaagtgggaacaaattctacctaatgaaaagatcattgttagccctatgccgtacccggagaagaatcttccgcttcgtatgattgctcggtctgaatccgccaccctcgcttaa

SEQ ID NO: 4 - protein, 502 aa, synthetic construct
MELITLLSALLVLAIVSLSTFFVLYYNTPTKDGKTLPPGRMGWPFIGESYDFFAAGWKGKPESFIFDRLKKFAKGNLNGQFRTSLFGNKSIVVAGAAANKLLFSNEKKLVTMWWPPSIDKAFPSTAQLSANEEALLMRKFFPSFLIRREALQRYIPIMDDCTRRHFATGAWGPSDKIEAFNVTQDYTFWVACRVFMSIDAQEDPETVDSLFRHFNVLKAGIYSMHIDLPWTNFHHAMKASHAIRSAVEQIAKKRRAELAEGKAFPTQDMLSYMLETPITSAEDSKDGKAKYLNDADIGTKILGLLVGGHDTSSTVIAFFFKFMAENPHVYEAIYKEQMEVAATKAPGELLNWDDLQKMKYSWCAICEVMRLTPPVQGAFRQAITDFTHNGYLIPKGWKIYWSTHSTHRNPEIFPQPEKFDPTRFEGNGPPAFSFVPFGGGPRMCPGKEYARLQVLTFVHHIVTKFKWEQILPNEKIIVSPMPYPEKNLPLRMIARSESATLA

SEQ ID NO: 5 - DNA, 1569 bp, synthetic construct
  source 1..1569: mol_type = other DNA
atggagtatttgccgtacattgcaacatcaattgcgtgcatagtaatactaagatgggcattgaacatgatgcaatggctatggttcgaaccgaggcggttggagaaattacttagaaaacaaggacttcaaggaaattcatataagtttttatttggagatatgaaggaaagttctatgttgagaaatgaagctttagcaaagcctatgcctatgccttttgataatgactactttcctcgtattaatccttttgttgatcaacttcttaacaaatatggtatgaattgtttcttgtggatggggcctgttccggctattcaaatcggagaaccagagttagttagggaagctttcaaccggatgcacgagtttcaaaagcccaaaactaaccctttgagtgctttactcgccaccggacttgttagctacgagggcgacaaatgggccaagcaccgccgccttatcaacccctcttttcatgttgaaaagctcaagcttatgattcctgcattccgcgagagcattgtggaggtggtcaatcaatgggagaagaaagtacctgaaaacggctctgctgaaatagatgtatggccgtctcttactagtttaaccggagatgttatctcaagagctgcctttggcagcgtgtatggcgatggaagaaggattttcgaacttctagctgttcagaaagaactcgttttaagtctgctcaagttttcgtacatccctggatacacgtatttgccaacagagggaaacaagaagatgaaggcggtgaacaatgagatacaaagactactcgaaaacgtgattcaaaacagaaagaaggcgatggaagccggagaagcagcaaaagatgatctgttgggtttactgatggattccaattacaaggagagtatgcttgaaggcggcgggaaaaacaaaaaattgatcatgagttttcaagatcttattgacgagtgtaagctcttcttcttagctgggcacgagacgactgctgtgttacttgtgtggactttgattttgttgtgtaagcaccaagactggcaaaccaaagctcgcgaagaagttttggctacttttggaatgtcggaacccactgattatgatgccttaaaccgtctcaagattgtgacaatgatactaaatgaggtcctaagattgtacccaccggttgtttcaaccaaccgaaaactattcaagggcgaaacaaaactcggaaacttggtaataccaccaggtgtcggtatctcactattaaccatccaagcaaaccgtgacccgaaagtttggggggaggatgcaagtgagttccgacctgatagatttgcagaagggctagtgaaggcgactaagggcaatgtcgcgtttttccccttcggttggggtcctaggatttgtattggccaaaattttgcgctgaccgagtcaaagatggcggttgctatgatattgcaacgcttcactttcgacctttcaccgtcttacactcatgctccgtcgggccttattactcttaacccgcaatatggggctcctctcatgtttcgtagacgttaa

SEQ ID NO: 6 - protein, 522 aa, synthetic construct
MEYLPYIATSIACIVILRWALNMMQWLWFEPRRLEKLLRKQGLQGNSYKFLFGDMKESSMLRNEALAKPMPMPFDNDYFPRINPFVDQLLNKYGMNCFLWMGPVPAIQIGEPELVREAFNRMHEFQKPKTNPLSALLATGLVSYEGDKWAKHRRLINPSFHVEKLKLMIPAFRESIVEVVNQWEKKVPENGSAEIDVWPSLTSLTGDVISRAAFGSVYGDGRRIFELLAVQKELVLSLLKFSYIPGYTYLPTEGNKKMKAVNNEIQRLLENVIQNRKKAMEAGEAAKDDLLGLLMDSNYKESMLEGGGKNKKLIMSFQDLIDECKLFFLAGHETTAVLLVWTLILLCKHQDWQTKAREEVLATFGMSEPTDYDALNRLKIVTMILNEVLRLYPPVVSTNRKLFKGETKLGNLVIPPGVGISLLTIQANRDPKVWGEDASEFRPDRFAEGLVKATKGNVAFFPFGWGPRICIGQNFALTESKMAVAMILQRFTFDLSPSYTHAPSGLITLNPQYGAPLMFRRR

SEQ ID NO: 7 - DNA, 2283 bp, synthetic construct
  source 1..2283: mol_type = other DNA
atgtggaggttaaaaatagcagaaggtggaaatgacccgtatttgtatagcacaaacaattttgtaggacgtcaaacttgggaatttgatagcgagtacggtactcctgaagctataaaagaagtagaagaagctcgacaaattttttacaaaaatcgatttcaagttaagccttgtggcgatcttctatggcgttttcagttcctaagagagaaaaacttcaagcaaacaataccgcaagtgaaggtgggtgatggggaggaggtcacctacgaagccgcctcaacgacgttaaagcgttccgtcaacttactcacggccctgcaggccgacgacggtcactggcctgctgaaattgctggccctcaatttttcctccctcctttggtgttttgcttgtacatcaccggacatctcaacgttgttttcaatgttcatcaccgtgaagaaattcttcgtagcatttattatcaccagaatgaggatggagggtgggggttgcacattgaaggacacagcaccatgttctgtacggcgttgaactacatatgtttgcggatgctaggagtcggtcctgatgaaggagacgacaacgcttgccctagggctcgtaaatggatcctcgaccatggtagtgtcactcatatcccttcttggggaaagacttggctttctatactcggtttgtttgattggtccggaagtaacccgatgccacctgagttttggattctgcctactttcatgcctatgtatccagcgaaaatgtggtgttactgtcgaatggtgtacatgccgatgtcgtacttatacgggaagaggttcgttggtccgattacacctctaatcaaacagctcagagaggaacttttcagtgaaccgtttgaagaaatcaagtggaaaaaagtccgtcatctgtgtgcaccggaggatctctactacccgcatccattgattcaagacttaatgtgggacagtctttacttattcaccgagcctcttcttactcgctggccgttcaacaatttgatacgacagaaggccttacaagtgacgatggatcatatacattacgaagatgagaacagtcgatacataaccataggatgcgttgaaaaggttttgtgtatgttggcctgttgggttgaagacccaaatggtgtttgttacaaaaaacatcttgctagagttcccgattatatatggattgccgaggatggccttaaaatgcagagttttggaagtcaacagtgggactgtggctttgctgtgcaagcattactagcttcgaatatgagtcttgatgaaatcggacctgcccttaagaaaggccacttctttatcaaagagtctcaggtgaaagataatccctcgggtgatttcaagagcatgcaccgtcatatctcgaagggatcgtggacgttttctgaccaagatcatggttggcaggtctctgactgcactgcagaaggccttaagtgctgcttgatcttatcaaccatgccgccagaaattgttggagaaaagatggaccctgagaggctctacgactctgtcaatgtcctgctttctctacagagtgaaaatggaggtctatctgcttgggaaccagctggagcacaagcttggttagagcttctaaatccaacggaattcttcgcagacattgtgatcgagcatgagtatgttgaatgtactggtgcatcaattcaagctctggtattattcaagaaaatgtaccctggtcaccgaaagaaagagatcgaaaatttcatagccaaggccgcgaaatacctcgaggacacccaatatccaaacggctcttggtatggaaattggggtgtgtgtttcacgtatgggacgtggtttgcgctaggagggctagcggcagcgggcaaaacatacgcgaattgtgctgcgatgcgaaaaggtgttgaattccttcttaagtcacaaaaggaggacggtgggtggggcgaaagctatgtttcatgcccgaaaaaggacttcgtgccgctggaaggaccatccaatctaactcaaaccgcatgggcgttgatgggtctaatttacgcacgacagatggagagggatccgacaccgctacaccaagcagcaaagcttttgatcaattcacaactcgaaaacggagatttccctcaacaggaaataacaggagtattcatgaagaattgcatgctacactatccaatgtacaggactatttatccactgtgggctattgcagaatataggacgcatgttcctttgaggcttagttaa

SEQ ID NO: 8 - protein, 760 aa, synthetic construct
MWRLKIAEGGNDPYLYSTNNFVGRQTWEFDSEYGTPEAIKEVEEARQIFYKNRFQVKPCGDLLWRFQFLREKNFKQTIPQVKVGDGEEVTYEAASTTLKRSVNLLTALQADDGHWPAEIAGPQFFLPPLVFCLYITGHLNVVFNVHHREEILRSIYYHQNEDGGWGLHIEGHSTMFCTALNYICLRMLGVGPDEGDDNACPRARKWILDHGSVTHIPSWGKTWLSILGLFDWSGSNPMPPEFWILPTFMPMYPAKMWCYCRMVYMPMSYLYGKRFVGPITPLIKQLREELFSEPFEEIKWKKVRHLCAPEDLYYPHPLIQDLMWDSLYLFTEPLLTRWPFNNLIRQKALQVTMDHIHYEDENSRYITIGCVEKVLCMLACWVEDPNGVCYKKHLARVPDYIWIAEDGLKMQSFGSQQWDCGFAVQALLASNMSLDEIGPALKKGHFFIKESQVKDNPSGDFKSMHRHISKGSWTFSDQDHGWQVSDCTAEGLKCCLILSTMPPEIVGEKMDPERLYDSVNVLLSLQSENGGLSAWEPAGAQAWLELLNPTEFFADIVIEHEYVECTGASIQALVLFKKMYPGHRKKEIENFIAKAAKYLEDTQYPNGSWYGNWGVCFTYGTWFALGGLAAAGKTYANCAAMRKGVEFLLKSQKEDGGWGESYVSCPKKDFVPLEGPSNLTQTAWALMGLIYARQMERDPTPLHQAAKLLINSQLENGDFPQQEITGVFMKNCMLHYPMYRTIYPLWAIAEYRTHVPLRLS

SEQ ID NO: 9 - DNA, 2097 bp, synthetic construct
  source 1..2097: mol_type = other DNA
atgtcaccccacaacacctgcactctacaaataacccgagccctcctcagccgcctccacatcctcttccactccgccctcgtcgcctccgtcttctactaccgcttttccaacttctcctctggcccggcatgggccctcatgactttcgccgagctcaccctcgccttcatctgggccctcacccaggccttccgctggcggcccgtcgtccgggccgtcttcgggcccgaggagattgacccggcccagctcccgggtctggacgtgttcatatgcacggcagacccgaggaaggagccggtgatggaggtgatgaactcggtggtgtcggcattggcgttggattatccggcagagaagctggcggtttacttgtcggacgacggcgggtcgcccttgactagggaggttattagggaggctgccgtgtttgggaagtactgggtcgggttttgtgggaagtataatgttaagacgaggtgtcctgaggcctattttagttcgttttgtgatggtgaaagagttgatcataatcaggattatttgaacgacgagctttccgtcaagtcgaaatttgaagcgtttaagaagtatgtgcaaaaagcaagtgaagacgccaccaaatgtattgttgtcaatgatcgtccttcttgtgttgagattattcatgacagcaagcagaacggagagggtgaagtgaaaatgccgcttcttgtttacgtagccagggaaaaaagaccgggttttaatcaccatgctaaagccggagccattaatacacttcttcgagtgtcgggtttactgagcaatagccctttctttttggtgttggattgtgatatgtactgtaatgatccaacgtctgcgcgtcaagctatgtgcttccatcttgacccgaaactagctccctctctcgcgtttgtgcaataccctcaaattttctacaacaccagcaaaaacgacatctatgatggtcaggccagagcagcttttaagactaaatatcaaggcatggatggtcttagagggccggttatgagtggcacggggtatttcttgaagaggaaagcattgtacggaaaaccacacgaccaagatgaattactcagggagcagccaacgaaggcctttggctcctctaagatattcatcgcgtcccttggtgaaaatacctgtgttgccttgaaaggattgagtaaagacgagttgttgcaagagactcaaaaattggctgcttgtacatacgaatcaaacacgttatggggtagcgaggttggatactcgtacgactgcttgttggagagcacatactgtgggtacttattacactgcaaaggatggatctcagtatatctatacccgaaaaagccgtgtttcttggggtgtgcaacagtggacatgaatgatgccatgcttcagataatgaaatggacttctggattgattggcgttggcatatcaaagttcagcccgttcacatacgccatgtctcggatctccattatgcaaagtctttgctatgcttacttcgctttttcgggcctatttgctgtcttcttcttgatctatggcgttgttcttccgtattccctcttgcagggtgttccgctcttccccaaggcaggagatccatggcttttggcatttgcgggagtattcatatcctcgcttcttcagcacctgtacgaggttctctcaagcggagaaacagtgaaagcgtggtggaacgagcaaagaatctggatcataaaatcaatcaccgcctgtctgtttggtcttctggacgctatgcttaacaaaattggcgtcttaaaggctagtttcagactgacaaacaaggctgtcgacaaacaaaaactcgataaatacgagaagggcaggttcgatttccaaggcgcacaaatgttcatggtccctctcatgattctggtggtattcaatttggtctcgttctttggcggcttaagaagaaccgtcattcataaaaactacgaagacatgttcgcgcagcttttcctctcgttgttcattctagctcttagctatcctatcatggaggagattgtccgaaaagctagaaaaggtcgctcttaa

SEQ ID NO: 10 - protein, 698 aa, synthetic construct
MSPHNTCTLQITRALLSRLHILFHSALVASVFYYRFSNFSSGPAWALMTFAELTLAFIWALTQAFRWRPVVRAVFGPEEIDPAQLPGLDVFICTADPRKEPVMEVMNSVVSALALDYPAEKLAVYLSDDGGSPLTREVIREAAVFGKYWVGFCGKYNVKTRCPEAYFSSFCDGERVDHNQDYLNDELSVKSKFEAFKKYVQKASEDATKCIVVNDRPSCVEIIHDSKQNGEGEVKMPLLVYVAREKRPGFNHHAKAGAINTLLRVSGLLSNSPFFLVLDCDMYCNDPTSARQAMCFHLDPKLAPSLAFVQYPQIFYNTSKNDIYDGQARAAFKTKYQGMDGLRGPVMSGTGYFLKRKALYGKPHDQDELLREQPTKAFGSSKIFIASLGENTCVALKGLSKDELLQETQKLAACTYESNTLWGSEVGYSYDCLLESTYCGYLLHCKGWISVYLYPKKPCFLGCATVDMNDAMLQIMKWTSGLIGVGISKFSPFTYAMSRISIMQSLCYAYFAFSGLFAVFFLIYGVVLPYSLLQGVPLFPKAGDPWLLAFAGVFISSLLQHLYEVLSSGETVKAWWNEQRIWIIKSITACLFGLLDAMLNKIGVLKASFRLTNKAVDKQKLDKYEKGRFDFQGAQMFMVPLMILVVFNLVSFFGGLRRTVIHKNYEDMFAQLFLSLFILALSYPIMEEIVRKARKGRS

SEQ ID NO: 11 - DNA, 1479 bp, synthetic construct
  source 1..1479: mol_type = other DNA
atgggttcaaatacagaagcaactgaaatacccaaaatgcccttgaaaatagtcttccttacacttcctatagccggacacatgctccacattgtagacaccgcaagcacatttgccatacatggagtcgagtgtaccataatcactacccctgcaaatgtccctttcatcgaaaaatcaatctctgcaaccaacaccacaattcgacagttcctcagtatccgcctcgtcgatttcccccatgaagctgtcggccttcctcccggtgtcgaaaacttcagtgcagtcacgtgtccggatatgagacccaaaatatcgaaaggactttcgatcatacaaaaaccaactgaagacttaatcaaggaaatatcacctgattgtattgtttctgacatgttttacccttggacttctgatttcgcccttgaaataggtgttccaagggtggtttttcgcggttgtgggatgtttcccatgtgttgttggcatagtattaagtcacatttaccacatgagaaggttgacagagatgatgaaatgattgttcttcctacattgcctgatcatatagagatgagaaaatctacattacctgattgggtaaggaaaccaactgggtacagttatttgatgaagatgattgatgcggccgaattgaagagttatggagtaattgttaatagttttagtgatttagagagggattatgaggagtattttaagaatgtcaccgggttaaaggtgtggaccgtcggtccgatttcgttacatgtgggtcggaatgaggagttagaagggtcagatgagtgggtcaaatggctagatgggaaaaaactagactcggttatttatgttagttttggtggggtggcgaagtttccaccccaccagctgagagaaatcgcggccggattagaatcatctggccacgattttgtttgggtggtgagggcgagtgacgaaaatggcgaccaagctgaagcggatgagtggtccctacaaaaatttaaagagaaaatgaagaaaactaaccatgggttggttatagagagttgggtcccacaacttatgtttttggaacataaggctatcggaggaatgttgacacatgttggttggggtacaatgttggaagggattacagcgggtttaccgttggtgacgtggccattgtatgccgagcagttttacaatgagaggttggtggttgatgtgttgaagattggagttggtgttggggtgaaagagttctgtgggttggatgatattggcaagaaggagaccattggtagggagaatatcgaggcatcggtgagattagtgatgggcgatggcgaggaggcggctgccatgagactgcgggtgaaggagttgagtgaggcgtctatgaaggcggttcgagaaggtggttcatctaaggctaatatacacgatttccttaacgagctgtctacgttgagatcgttaaggcaggcttga

SEQ ID NO: 12 - protein, 492 aa, synthetic construct
MGSNTEATEIPKMPLKIVFLTLPIAGHMLHIVDTASTFAIHGVECTIITTPANVPFIEKSISATNTTIRQFLSIRLVDFPHEAVGLPPGVENFSAVTCPDMRPKISKGLSIIQKPTEDLIKEISPDCIVSDMFYPWTSDFALEIGVPRVVFRGCGMFPMCCWHSIKSHLPHEKVDRDDEMIVLPTLPDHIEMRKSTLPDWVRKPTGYSYLMKMIDAAELKSYGVIVNSFSDLERDYEEYFKNVTGLKVWTVGPISLHVGRNEELEGSDEWVKWLDGKKLDSVIYVSFGGVAKFPPHQLREIAAGLESSGHDFVWVVRASDENGDQAEADEWSLQKFKEKMKKTNHGLVIESWVPQLMFLEHKAIGGMLTHVGWGTMLEGITAGLPLVTWPLYAEQFYNERLVVDVLKIGVGVGVKEFCGLDDIGKKETIGRENIEASVRLVMGDGEEAAAMRLRVKELSEASMKAVREGGSSKANIHDFLNELSTLRSLRQA

SEQ ID NO: 13 - DNA, 1464 bp, synthetic construct
  source 1..1464: mol_type = other DNA
atgaagtcaccactaaagttgtacttcctgccatacatatcaccaggccatatgatcccactttccgaaatggctcggttattcgccaaccaagggcaccacgtgaccatcatcaccaccacctcgaacgccaccctcctccaaaaatacaccaccgccaccctgtctctacatcttattcccctccctaccaaagaggccggccttccagacggcctcgaaaacttcatttctgtcaacgatcttgaaaccgctggcaaactctactacgctctttccctcctgcaacccgtcattgaggagtttatcacgtctaacccgcccgattgtatcgtgtccgacatgttctatccctggactgcggacctggcgtcccaactccaggtcccgcgtatggtctttcatgcagcgtgtatattcgctatgtgcatgaaagagtcaatgcggggccctgacgccccgcatctgaaggtcagctctgattatgagctgtttgaagtcaaggggctaccggacccggtttttatgacccgggcccagctccctgactacgtgcgtaccccaaacgggtacacacagctcatggagatgtggcgagaagcggaaaagaaaagttacggtgttatggttaataatttttacgaacttgacccggcttataccgagcattatagtaagattatgggccataaggtctggaatattgggcctgcggcccaaattcttcaccgtggttctggtgataaaatcgagagggttcacaaagccgttgttggtgaaaaccaatgcttgagttggctcgacactaaggaacctaactcggttttttacgtctgctttgggagcgcgattaggttccctgatgatcagctctacgaaattgctagcgcgctagaatcatctggcgcgcagtttatatgggccgttcttggaaaagactcggataattcagactcgaactcagactcagaatggctgcctgcagggttcgaggaaaaaatgaaggaaacgggtagagggatgataatacgaggttgggccccacaggtgttgatattggaccacccgtctgtaggcgggtttatgactcactgtggctggaactcgacaattgagggggttagcgcgggggtggggatggtgacatggccgttgtatgcggaacaattttacaatgagaagttaataacacaagtgcttaagataggggtggaggccggggtggaggagtggaacttgtgggtggatgttgggaggaaattggtgaagagagagaagatcgaggcggcaattagggcggtgatgggtgaggccggggtggagatgaggaggaaggcgaaagagttgagtgtcaaggctaagaaggcggtgcaggatggtgggtcgtctcaccgtaatttaatggctttgatcgaagatctgcagaggattagagatgataaaatgagtaaggttgctaattag

SEQ ID NO: 14 - protein, 487 aa, synthetic construct
MKSPLKLYFLPYISPGHMIPLSEMARLFANQGHHVTIITTTSNATLLQKYTTATLSLHLIPLPTKEAGLPDGLENFISVNDLETAGKLYYALSLLQPVIEEFITSNPPDCIVSDMFYPWTADLASQLQVPRMVFHAACIFAMCMKESMRGPDAPHLKVSSDYELFEVKGLPDPVFMTRAQLPDYVRTPNGYTQLMEMWREAEKKSYGVMVNNFYELDPAYTEHYSKIMGHKVWNIGPAAQILHRGSGDKIERVHKAVVGENQCLSWLDTKEPNSVFYVCFGSAIRFPDDQLYEIASALESSGAQFIWAVLGKDSDNSDSNSDSEWLPAGFEEKMKETGRGMIIRGWAPQVLILDHPSVGGFMTHCGWNSTIEGVSAGVGMVTWPLYAEQFYNEKLITQVLKIGVEAGVEEWNLWVDVGRKLVKREKIEAAIRAVMGEAGVEMRRKAKELSVKAKKAVQDGGSSHRNLMALIEDLQRIRDDKMSKVAN

SEQ ID NO: 15 - DNA, 1407 bp, synthetic construct
  source 1..1407: mol_type = other DNA
atgtcggatcaaaatgataaaaaggtcgaaataatagtatttccataccatggccaaggtcacatgaacaccatgctacaattcgccaaacgaattgcgtggaaaaacgccaaagttacaatcgctacgacattgtccaccactaataaaatgaagtccaaggtcgagaatgcctggggcacttctataaccttggactccatttacgatgactctgacgagtcgcagataaaattcatggaccgtatggccaggtttgaggctgctgcagcctcgagcctgtccaaactcctggtccagaaaaaagaagaagctgacaacaaagtcttgttggtttacgacgggaatttgccgtgggcgctggatatcgcccacgagcatggcgtgcgtggggccgcgttttttccacagtcgtgtgcgacggtcgccacgtactactcgttgtatcaagagacgcaggggaaggagctagagacggagttgccggcggtgtttccgccgttggagttgatacaacggaatgtaccgaatgtgtttggattgaagtttccggaggcggttgtggctaagaatgggaaggagtatagtccttttgtgttgtttgtgttgaggcagtgtattaaccttgagaaggctgatttgctgcttttcaatcagtttgataagttggttgaacctggggaggttctgcaatggatgtcgaagatattcaacgtaaagacaatcggaccgacacttccatcttcatacatcgacaaacgaatcaaagacgacgtggactacggtttccacgcattcaacctcgacaacaactcctgcatcaattggcttaactccaaacccgctcgctctgtcatctacatagcatttgggagcagcgtccactacagcgttgagcaaatgaccgaaatagccgaggccttaaagagccaaccgaacaatttcctttgggcagtccgagaaaccgaacaaaagaaactccctgaagacttcgtccaacaaacctcggaaaaagggttaatgctctcatggtgccctcaattagatgttttggtgcatgaatcaatcagttgttttgtgacacattgtggttggaactcgattacagaggcacttagcttcggggtaccaatgctgtcagtgccacagtttttggaccagcctgttgatgctcactttgtggaacaggtttggggtgctggaattacggtcaagaggagcgaagacggtttggttactcgagacgaaattgttcggtgcttggaggtgttaaataatggcgaaaaggcggaggaaattaaggcgaatgtggcgaggtggaaggttttggctaaggaagctttggatgaaggtggtagttctgataagcacattgacgaaattattgagtgggtttcatctttctaa

SEQ ID NO: 16 - protein, 468 aa, synthetic construct
MSDQNDKKVEIIVFPYHGQGHMNTMLQFAKRIAWKNAKVTIATTLSTTNKMKSKVENAWGTSITLDSIYDDSDESQIKFMDRMARFEAAAASSLSKLLVQKKEEADNKVLLVYDGNLPWALDIAHEHGVRGAAFFPQSCATVATYYSLYQETQGKELETELPAVFPPLELIQRNVPNVFGLKFPEAVVAKNGKEYSPFVLFVLRQCINLEKADLLLFNQFDKLVEPGEVLQWMSKIFNVKTIGPTLPSSYIDKRIKDDVDYGFHAFNLDNNSCINWLNSKPARSVIYIAFGSSVHYSVEQMTEIAEALKSQPNNFLWAVRETEQKKLPEDFVQQTSEKGLMLSWCPQLDVLVHESISCFVTHCGWNSITEALSFGVPMLSVPQFLDQPVDAHFVEQVWGAGITVKRSEDGLVTRDEIVRCLEVLNNGEKAEEIKANVARWKVLAKEALDEGGSSDKHIDEIIEWVSSF

SEQ ID NO: 17 - DNA, 1344 bp, synthetic construct
  source 1..1344: mol_type = other DNA
atgtctgccaaaatgttgcacgtagttatgtacccatggttcgcatacggtcacatgatcccatttttacatttatcgaacaaattagccgaaaccggtcacaaagtcacgtacatactccccccaaaagcgctaacccgcttacaaaacctcaacctaaatccgacccaaatcacgttccggaccatcacggtcccccgagttgatgggttacccgctggtgccgagaacgtgaccgatattccggatattactctgcatactcatttggccacggcgctggatcgaacccgacccgaatttgagacgattgtcgagttgattaagccggatgtgataatgtatgacgtggcgtattgggtgccagaggtggcggtgaagtatggggcgaagagtgttgcgtatagtgtggtgtcggcggcaagtgtgtcgctgagtaagacggtggttgatcggatgacgccgttggagaaaccgatgacggaggaggagaggaagaagaagtttgctcagtatcctcacttaattcagctttatggtccttttggtgaaggtatcaccatgtacgaccgtctaacaggcatgcttagcaagtgtgacgctatagcttgtaggacctgccgtgagattgaaggcaagtattgccaatatttatccactcaatatgaaaagaaagtcacccttaccggcccggttcttcccgagccggaagtcggggccacactggaggccccttggtccgagtggcttagtcggttcaagcttggttcggttttattttgtgcctttgggagccaattttacttggacaaggaccagttccaggaaatcatcctcgggcttgaaatgacaaatttaccctttctgatggctgttcagccccctaagggttgcgccactatcgaggaggcgtaccctgaggggtttgctgagcgggtcaaggaccgaggagtcgtgacaagccagtgggtgcaacagctggttatactggcccacccagcggttgggtgctttgtgaaccattgcgcgtttgggacaatgtgggaggccttattgagcgaaaagcagttggtgatgatccctcaactaggtgaccaaatactgaacaccaaaatgttggccgatgaattgaaagtcggggttgaagtcgagagaggaatcggtgggtgggtgtctaaggagaatttgtgtaaggcgatcaagtccgtcatggacgaggatagtgaaattggcaaggacgtgaaacaaagtcatgaaaaatggagggcgactttgtcgagcaaagatttaatgtcgacttatattgatagtttcatcaaagatttacaagcactcgtcgagtga

SEQ ID NO: 18 - protein, 447 aa, synthetic construct
MSAKMLHVVMYPWFAYGHMIPFLHLSNKLAETGHKVTYILPPKALTRLQNLNLNPTQITFRTITVPRVDGLPAGAENVTDIPDITLHTHLATALDRTRPEFETIVELIKPDVIMYDVAYWVPEVAVKYGAKSVAYSVVSAASVSLSKTVVDRMTPLEKPMTEEERKKKFAQYPHLIQLYGPFGEGITMYDRLTGMLSKCDAIACRTCREIEGKYCQYLSTQYEKKVTLTGPVLPEPEVGATLEAPWSEWLSRFKLGSVLFCAFGSQFYLDKDQFQEIILGLEMTNLPFLMAVQPPKGCATIEEAYPEGFAERVKDRGVVTSQWVQQLVILAHPAVGCFVNHCAFGTMWEALLSEKQLVMIPQLGDQILNTKMLADELKVGVEVERGIGGWVSKENLCKAIKSVMDEDSEIGKDVKQSHEKWRATLSSKDLMSTYIDSFIKDLQALVE

SEQ ID NO: 19 - DNA, 1314 bp, synthetic construct
  source 1..1314: mol_type = other DNA
atgggtactaaagagttacacatagtaatgtacccatggctagcatttggtcatttcataccataccttcatctctctaacaaactcgctcaaaaaggccataaaatcactttcttacttcctcatagagccaaacttcaacttgactcccaaaatttatatccctcacttattaccctcgtaccaattaccgtcccacaggtcgacacccttcctctcggggccgaatcgactgctgatatcccccttagtcagcacggtgacctctccatcgccatggaccgtactcgacccgagattgagtctatcttgtctaaacttgacccaaaaccggacctgattttcttcgatatggcgcagtgggtgcctgtcatagcgtctaagcttgggatcaagtctgtttcgtataatatcgtttgcgccatttcgttggaccttgttcgagattggtataagaaggatgatggaagtaatgtgcctagttggacattgaagcatgacaagtcatcccatttcggggagaatattagtattctcgagcgagcgctgattgcgctcgggacgcctgatgccataggcatcaggtcgtgtcgggagatagagggggagtactgtgacagcatagcggaacgatttaagaaaccggtcttactaagcgggacgaccttacctgaaccatccgacgacccacttgacccaaaatgggtcaagtggctcggaaagttcgaggaaggttcggttattttttgctgcctagggagtcagcacgtgttagacaagccccagctccaggagctggcgctggggcttgaaatgacggggttgccattcttcctagcgattaaaccaccgctaggatacgcaaccctagacgaggtactacccgaggggttttcagaacgggttcgagatcgaggggtggctcatgggggatgggtacaacagcctcagatgctggcacacccttctgtagggtgctttttgtgtcactgtgggtcgtcgtcgatgtgggaggcattagtgagtgatacgcagctcgtattgtttcctcaaataccagatcaagctctaaacgcggttttaatggcggataaacttaaggtcggggtgaaggtcgagagagaggacgacggaggggtgtcgaaagaggtttggagtagagcaataaagagtgtgatggataaggagagtgaaattgctgcggaagtgaagaagaatcatactaagtggagagatatgttgattaatgaagaatttgtgaatgggtacattgacagtttcattaaggatctacaagatcttgttgagaagtag

SEQ ID NO: 20 - protein, 437 aa, synthetic construct
MGTKELHIVMYPWLAFGHFIPYLHLSNKLAQKGHKITFLLPHRAKLQLDSQNLYPSLITLVPITVPQVDTLPLGAESTADIPLSQHGDLSIAMDRTRPEIESILSKLDPKPDLIFFDMAQWVPVIASKLGIKSVSYNIVCAISLDLVRDWYKKDDGSNVPSWTLKHDKSSHFGENISILERALIALGTPDAIGIRSCREIEGEYCDSIAERFKKPVLLSGTTLPEPSDDPLDPKWVKWLGKFEEGSVIFCCLGSQHVLDKPQLQELALGLEMTGLPFFLAIKPPLGYATLDEVLPEGFSERVRDRGVAHGGWVQQPQMLAHPSVGCFLCHCGSSSMWEALVSDTQLVLFPQIPDQALNAVLMADKLKVGVKVEREDDGGVSKEVWSRAIKSVMDKESEIAAEVKKNHTKWRDMLINEEFVNGYIDSFIKDLQDLVEK

SEQ ID NO: 21 - DNA, 1473 bp, synthetic construct
  source 1..1473: mol_type = other DNA
atggaggaatcaaaggaggaagtacatgtagcattcttcccattcatgacaccaggtcactcaatcccaatgctagacttggtacgtttgttcattgctcgtggtgtcaaaactactgtcttcactactcctcttaatgctcctaatatttccaaatacctcaacattatccaagattcctcatcaaacaaaaacaccatttatgtaactccttttccttctaaagaagccggtttaccggaaggtgtggaaagccaggatagtaccacttcccccgaaatgaccctcaagttctttgttgctatggaattacttcaagacccccttgatgtttttttaaaagaaaccaaacctcattgtcttgttgctgataatttcttcccttacgccaccgacatcgcttctaagtatggcattcctaggtttgtttttcagttcactggcttctttcctatgtctgtcatgatggccttaaatcgtttccaccctcaaaactctgtatcatctgatgacgacccctttcttgttcccagtttaccccatgacatcaaattgactaagtcacaattgcaacgagagtacgagggtagtgatggtattgacaccgctctttctaggctctgtaatggcgccggtagagctttgtttactagttatggtgtcatttttaacagcttctaccaactcgaacctgattatgttgattattataccaacaccatggggaaacgatccagggtttggcatgtgggcccagtgtcgttatgcaaccgtcgacacgtggagggtaaatctggtagggggagaagtgcttcaattagtgagcatttgtgcttagagtggctcaatgccaaagaaccaaattcagtgatatatgtatgttttggtagtctcacatgtttctccaatgagcaactcaaagaaatcgcaaccgccttagaaaggtgtgaagagtattttatatgggtgttgaagggtggcaaagataatgagcaagagtggttgccacaagggtttgaagagagggttgaagggaaaggactaatcatacgggggtgggccccacaagtgttgattttagaccatgaagccataggcgggtttgtgacacactgtggttggaactcgacactagaaagtatatcagcgggggtgcccatggtgacatggcccatatatgcagagcaattttataatgagaaattggtgacggatgtactgaaggtgggggttaaagtagggtcaatgaagtggagtgagacgacgggggcgactcatttaaagcatgaggaaatagaaaaagcattgaagcaaataatggtgggagaagaggtgttagagatgagaaaaagagcaagtaagttgaaagagatggcttataatgctgttgaagaaggaggctcttcttattctcacctcacttccttaatcgacgaccttatggcttccaaagctgtgctacaaaaattttga

SEQ ID NO: 22 - protein, 490 aa, synthetic construct
MEESKEEVHVAFFPFMTPGHSIPMLDLVRLFIARGVKTTVFTTPLNAPNISKYLNIIQDSSSNKNTIYVTPFPSKEAGLPEGVESQDSTTSPEMTLKFFVAMELLQDPLDVFLKETKPHCLVADNFFPYATDIASKYGIPRFVFQFTGFFPMSVMMALNRFHPQNSVSSDDDPFLVPSLPHDIKLTKSQLQREYEGSDGIDTALSRLCNGAGRALFTSYGVIFNSFYQLEPDYVDYYTNTMGKRSRVWHVGPVSLCNRRHVEGKSGRGRSASISEHLCLEWLNAKEPNSVIYVCFGSLTCFSNEQLKEIATALERCEEYFIWVLKGGKDNEQEWLPQGFEERVEGKGLIIRGWAPQVLILDHEAIGGFVTHCGWNSTLESISAGVPMVTWPIYAEQFYNEKLVTDVLKVGVKVGSMKWSETTGATHLKHEEIEKALKQIMVGEEVLEMRKRASKLKEMAYNAVEEGGSSYSHLTSLIDDLMASKAVLQKF

SEQ ID NO: 23 - DNA, 1689 bp, synthetic construct
  source 1..1689: mol_type = other DNA
atggctgtggaggttcaccgccgggctcccgcgccccatggccggggcaccggggagaagggccgcgtgcaggccggggacgcgctgccgctgccgatccgccacaccaacctcatcttctcggcgctcttcgccgcctccctcgcatacctcatgcgccgctggagggagaagatccgcaactccacgccgctccacgtcgtggggctcaccgagatcttcgccatctgcggcctcgtcgcctccctcatctacctcctcagcttcttcggcatcgccttcgtgcagtccgtcgtatccaacagcgacgacgaggacgaggacttcctcatcgcggctgcagcatcccaggcccccccgccgccctcctccaagcccgcgccgcagcagtgcgccctgctgcagagcgccggagtcgcgcccgagaaaatgcccgaggaggacgaggaaatcgtcgccggggtcgtcgcagggaagatcccctcctacgtgctcgagaccaggctaggcgactgccgcagggcagccgggatccgccgcgaggcgctgcgccggatcaccggcagggagatcgacggccttcccctcgacggcttcgactacgactcgattctcggacagtgctgcgagatgcccgtcgggtacgtgcagctgccggtcggcgtcgcggggccgctcgtcctcgacggccgccgcatatacgtcccgatggccaccacggagggctgcctaatcgccagcaccaaccgcggatgcaaggccattgccgagtccggaggcgcatccagcgtcgtgtaccgcgacgggatgacccgcgcccccgtagcccgcttcccctccgcacgacgcgccgcagagctcaagggcttcctggagaatccggccaactacgacaccctgtccgtggtctttaacagatcaagcagatttgcaaggctgcagggggtcaagtgcgccatggctgggaggaacttgtacatgaggttcacctgcagcaccggggatgccatggggatgaacatggtctccaagggcgtccaaaatgtgctcgactatctgcaggaggacttccctgacatggacgttgtcagcatctcaggcaacttttgttccgacaagaaatcagctgctgtaaactggattgaaggccgtggaaagtccgtggtttgtgaggcagtaatcagagaggaagttgtccacaaggttctcaagaccaacgttcagtcactcgtggagttgaatgtgatcaagaaccttgctggctcagcagttgctggtgctcttgggggtttcaacgcccacgcaagcaacatcgtaacggctatcttcattgccactggtcaggatcctgcacagaatgtggagagctcacagtgtatcactatgttggaagctgtaaatgatggcagagaccttcacatctccgttacaatgccatctatcgaggtgggcacagttggtggaggcacgcagctggcctcacagtcggcctgcttggacctactgggcgtcaaaggcgccaacagggaatctccggggtcgaacgctaggctgctggccacggtggtggctggtgccgtcctagctggggagctgtccctcatctccgcccaagctgccggccatctggtccagagccacatgaaatacaacagatccagcaaggacatgtccaagatcgcctgctga

SEQ ID NO: 24 - protein, 562 aa, synthetic construct
MAVEVHRRAPAPHGRGTGEKGRVQAGDALPLPIRHTNLIFSALFAASLAYLMRRWREKIRNSTPLHVVGLTEIFAICGLVASLIYLLSFFGIAFVQSVVSNSDDEDEDFLIAAAASQAPPPPSSKPAPQQCALLQSAGVAPEKMPEEDEEIVAGVVAGKIPSYVLETRLGDCRRAAGIRREALRRITGREIDGLPLDGFDYDSILGQCCEMPVGYVQLPVGVAGPLVLDGRRIYVPMATTEGCLIASTNRGCKAIAESGGASSVVYRDGMTRAPVARFPSARRAAELKGFLENPANYDTLSVVFNRSSRFARLQGVKCAMAGRNLYMRFTCSTGDAMGMNMVSKGVQNVLDYLQEDFPDMDVVSISGNFCSDKKSAAVNWIEGRGKSVVCEAVIREEVVHKVLKTNVQSLVELNVIKNLAGSAVAGALGGFNAHASNIVTAIFIATGQDPAQNVESSQCITMLEAVNDGRDLHISVTMPSIEVGTVGGGTQLASQSACLDLLGVKGANRESPGSNARLLATVVAGAVLAGELSLISAQAAGHLVQSHMKYNRSSKDMSKIAC

SEQ ID NO: 25 - DNA, 1275 bp, synthetic construct
  source 1..1275: mol_type = other DNA
atggcgcccgagaaaatgcccgaggaggacgaggaaatcgtcgccggggtcgtcgcagggaagatcccctcctacgtgctcgagaccaggctaggcgactgccgcagggcagccgggatccgccgcgaggcgctgcgccggatcaccggcagggagatcgacggccttcccctcgacggcttcgactacgactcgattctcggacagtgctgcgagatgcccgtcgggtacgtgcagctgccggtcggcgtcgcggggccgctcgtcctcgacggccgccgcatatacgtcccgatggccaccacggagggctgcctaatcgccagcaccaaccgcggatgcaaggccattgccgagtccggaggcgcatccagcgtcgtgtaccgcgacgggatgacccgcgcccccgtagcccgcttcccctccgcacgacgcgccgcagagctcaagggcttcctggagaatccggccaactacgacaccctgtccgtggtctttaacagatcaagcagatttgcaaggctgcagggggtcaagtgcgccatggctgggaggaacttgtacatgaggttcacctgcagcaccggggatgccatggggatgaacatggtctccaagggcgtccaaaatgtgctcgactatctgcaggaggacttccctgacatggacgttgtcagcatctcaggcaacttttgttccgacaagaaatcagctgctgtaaactggattgaaggccgtggaaagtccgtggtttgtgaggcagtaatcagagaggaagttgtccacaaggttctcaagaccaacgttcagtcactcgtggagttgaatgtgatcaagaaccttgctggctcagcagttgctggtgctcttgggggtttcaacgcccacgcaagcaacatcgtaacggctatcttcattgccactggtcaggatcctgcacagaatgtggagagctcacagtgtatcactatgttggaagctgtaaatgatggcagagaccttcacatctccgttacaatgccatctatcgaggtgggcacagttggtggaggcacgcagctggcctcacagtcggcctgcttggacctactgggcgtcaaaggcgccaacagggaatctccggggtcgaacgctaggctgctggccacggtggtggctggtgccgtcctagctggggagctgtccctcatctccgcccaagctgccggccatctggtccagagccacatgaaatacaacagatccagcaaggacatgtccaagatcgcctgctga

SEQ ID NO: 26 - protein, 424 aa, synthetic construct
MAPEKMPEEDEEIVAGVVAGKIPSYVLETRLGDCRRAAGIRREALRRITGREIDGLPLDGFDYDSILGQCCEMPVGYVQLPVGVAGPLVLDGRRIYVPMATTEGCLIASTNRGCKAIAESGGASSVVYRDGMTRAPVARFPSARRAAELKGFLENPANYDTLSVVFNRSSRFARLQGVKCAMAGRNLYMRFTCSTGDAMGMNMVSKGVQNVLDYLQEDFPDMDVVSISGNFCSDKKSAAVNWIEGRGKSVVCEAVIREEVVHKVLKTNVQSLVELNVIKNLAGSAVAGALGGFNAHASNIVTAIFIATGQDPAQNVESSQCITMLEAVNDGRDLHISVTMPSIEVGTVGGGTQLASQSACLDLLGVKGANRESPGSNARLLATVVAGAVLAGELSLISAQAAGHLVQSHMKYNRSSKDMSKIAC

SEQ ID NO: 27 - DNA, 1212 bp, synthetic construct
  source 1..1212: mol_type = other DNA
atgggggcgctgtcgcggccggaggaggtggtggcgctggtcaagctgagggtggcggcggggcagatcaagcgccagatcccggccgaggaacactgggccttcgcctacgacatgctccagaaggtctcccgcagcttcgcgctcgtcatccagcagctcggacccgaactccgcaatgccgtgtgcatcttctacctcgtgctccgggccctggacaccgtcgaggacgacaccagcatccccaacgacgtgaagctgcccatccttcgggatttctaccgccatgtctacaaccccgactggcgttattcatgtggaacaaaccactacaaggtgctgatggataagttcagactcgtctccacggctttcctggagctaggcgaaggatatcaaaaggcaattgaagaaatcactaggcgaatgggagcaggaatggcaaaatttatatgccaggaggttgaaacgattgatgactataatgagtactgccactatgtagcagggctagtaggctatggactttccaggctctttcatgctgctgggacagaagatctggcttcagatcaactttcgaattcaatgggtttgtttcttcagaaaaccaatataataagggattatttggaggatataaatgagataccaaagtgccgtatgttttggcctcgagaaatatggagtaaatatgcagataaacttgaggacctcaagtatgaggaaaattcagaaaaagcagtgcaatgcttgaatgatatggtgactaatgctttggtccacgccgaagactgtcttcaatacatgtctgcgttgaaggataatactaattttcggttttgtgcaatacctcagataatggcaattgggacatgtgctatttgctacaataatgtgaaagtctttagaggagttgttaagatgaggcgtgggctcactgcacgaataattgatgagacaaaatcaatgtcagatgtctattctgctttctatgagttctcttcattgctagagtcaaagattgacgataacgacccaagttctgcactaacacggaagcgtgtagaggcaataaagaggacttgcaagtcatccggtttactaaagagaaggggatacgacctggaaaagtcaaagtataggcatatgttgatcatgcttgcacttctgttggtggctattatcttcggtgtactgtacgccaagtga

SEQ ID NO: 28 - protein, 403 aa, synthetic construct
MGALSRPEEVVALVKLRVAAGQIKRQIPAEEHWAFAYDMLQKVSRSFALVIQQLGPELRNAVCIFYLVLRALDTVEDDTSIPNDVKLPILRDFYRHVYNPDWRYSCGTNHYKVLMDKFRLVSTAFLELGEGYQKAIEEITRRMGAGMAKFICQEVETIDDYNEYCHYVAGLVGYGLSRLFHAAGTEDLASDQLSNSMGLFLQKTNIIRDYLEDINEIPKCRMFWPREIWSKYADKLEDLKYEENSEKAVQCLNDMVTNALVHAEDCLQYMSALKDNTNFRFCAIPQIMAIGTCAICYNNVKVFRGVVKMRRGLTARIIDETKSMSDVYSAFYEFSSLLESKIDDNDPSSALTRKRVEAIKRTCKSSGLLKRRGYDLEKSKYRHMLIMLALLLVAIIFGVLYAK

SEQ ID NO: 29 - DNA, 2325 bp, synthetic construct
  source 1..2325: mol_type = other DNA
atgaaaaacatgatgaattataaattaaaactctgttctgtctcaaaaaactcaaaaggagtctctctctcacctacaccacacctaaccaaaccccctacgattcacacagagagagatcttcttcttccttcttcttccttcttctttcttcttctttcttcttctagctacaacatctacaacgccatgtcctcttcttcttcttcgtcaacctccatgatcgatctcatggcagcaatcatcaaaggagagcctgtaattgtctccgacccagctaatgcctccgcttacgagtccgtagctgctgaattatcctctatgcttatagagaatcgtcaattcgccatgattgttaccacttccattgctgttcttattggttgcatcgttatgctcgtttggaggagatccggttctgggaattcaaaacgtgtcgagcctcttaagcctttggttattaagcctcgtgaggaagagattgatgatgggcgtaagaaagttaccatctttttcggtacacaaactggtactgctgaaggttttgcaaaggctttaggagaagaagctaaagcaagatatgaaaagaccagattcaaaatcgttgatttggatgattacgcggctgatgatgatgagtatgaggagaaattgaagaaagaggatgtggctttcttcttcttagccacatatggagatggtgagcctaccgacaatgcagcgagattctacaaatggttcaccgaggggaatgacagaggagaatggcttaagaacttgaagtatggagtgtttggattaggaaacagacaatatgagcattttaataaggttgccaaagttgtagatgacattcttgtcgaacaaggtgcacagcgtcttgtacaagttggtcttggagatgatgaccagtgtattgaagatgactttaccgcttggcgagaagcattgtggcccgagcttgatacaatactgagggaagaaggggatacagctgttgccacaccatacactgcagctgtgttagaatacagagtttctattcacgactctgaagatgccaaattcaatgatataaacatggcaaatgggaatggttacactgtgtttgatgctcaacatccttacaaagcaaatgtcgctgttaaaagggagcttcatactcccgagtctgatcgttcttgtatccatttggaatttgacattgctggaagtggacttacgtatgaaactggagatcatgttggtgtactttgtgataacttaagtgaaactgtagatgaagctcttagattgctggatatgtcacctgatacttatttctcacttcacgctgaaaaagaagacggcacaccaatcagcagctcactgcctcctcccttcccaccttgcaacttgagaacagcgcttacacgatatgcatgtcttttgagttctccaaagaagtctgctttagttgcgttggctgctcatgcatctgatcctaccgaagcagaacgattaaaacaccttgcttcacctgctggaaaggatgaatattcaaagtgggtagtagagagtcaaagaagtctacttgaggtgatggccgagtttccttcagccaagccaccacttggtgtcttcttcgctggagttgctccaaggttgcagcctaggttctattcgatatcatcatcgcccaagattgctgaaactagaattcacgtcacatgtgcactggtttatgagaaaatgccaactggcaggattcataagggagtgtgttccacttggatgaagaatgctgtgccttacgagaagagtgaaaactgttcctcggcgccgatatttgttaggcaatccaacttcaagcttccttctgattctaaggtaccgatcatcatgatcggtccagggactggattagctccattcagaggattccttcaggaaagactagcgttggtagaatctggtgttgaacttgggccatcagttttgttctttggatgcagaaaccgtagaatggatttcatctacgaggaagagctccagcgatttgttgagagtggtgctctcgcagagctaagtgtcgccttctctcgtgaaggacccaccaaagaatacgtacagcacaagatgatggacaaggcttctgatatctggaatatgatctctcaaggagcttatttatatgtttgtggtgacgccaaaggcatggcaagagatgttcacagatctctccacacaatagctcaagaacaggggtcaatggattcaactaaagcagagggcttcgtgaagaatctgcaaacgagtggaagatatcttagagatgtatggtaa

SEQ ID NO: 30 - protein, 774 aa, synthetic construct
MKNMMNYKLKLCSVSKNSKGVSLSPTPHLTKPPTIHTERDLLLPSSSFFFLLLSSSSYNIYNAMSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 31 - DNA, 909 bp, synthetic construct
  source 1..909: mol_type = other DNA
atggctgaagcatcctcatttcttgcacagaaaaggtatgcggtcgtgacaggagcaaacaaaggactaggactagaaatatgcggacagcttgcttcacagggggtgacggtactgctgacatccagagatgaaaaacgaggcttagaagccattgaggagcttaagaaatcggggattaattcggaaaatcttgaatatcatcagctggatgttactaagccagctagtttcgcttctctggccgatttcatcaaggccaaatttggcaagcttgatatcctggtgaacaatgcagggatcagcggtgttattgtagattatgcagctttaatggaagccattcgccgtcgaggggcagagatcaattacgatggagtgatgaaacagacctacgagctagcagaggaatgcttgcaaacaaattactatggtgtgaaaagaaccattaatgctctccttccgctacttcagttttccgattcaccaaggatcgtcaatgtttcctccgatgttggcctccttaagaaaatacccggcgagagaatcagagaagccttaggcgacgtggaaaaacttacggaagaaagcgtggacgggattttagacgagtttctaagagatttcaaggaaggcaagatcgcagagaaaggttggcctacgtttaagagcgcctattcaatctcaaaggcggcgctcaattcgtacacgagggttttagcacggaaatacccgtcgatcatcatcaactgtgtctgcccgggtgtcgtcaaaaccgatatcaatcttaaaatgggccacttgacggttgaagaaggcgcggccagtcccgtgaggttagcactcatgccccttggttcgccttccggcctgttctatactcgaaacgaagtaactccatttgaatga

SEQ ID NO: 32 - protein, 302 aa, synthetic construct
MAEASSFLAQKRYAVVTGANKGLGLEICGQLASQGVTVLLTSRDEKRGLEAIEELKKSGINSENLEYHQLDVTKPASFASLADFIKAKFGKLDILVNNAGISGVIVDYAALMEAIRRRGAEINYDGVMKQTYELAEECLQTNYYGVKRTINALLPLLQFSDSPRIVNVSSDVGLLKKIPGERIREALGDVEKLTEESVDGILDEFLRDFKEGKIAEKGWPTFKSAYSISKAALNSYTRVLARKYPSIIINCVCPGVVKTDINLKMGHLTVEEGAASPVRLALMPLGSPSGLFYTRNEVTPFE

SEQ ID NO: 33 - DNA, 1350 bp, synthetic construct
  source 1..1350: mol_type = other DNA
atggttcttagtcgattggattttccgtccgatttcatttttggctccggcacgtcagcttctcaggtagaaggtgcagcactagaggatgggaagacttcgactgcatttgaaggattcttaactcgcatgagtggaaatgatttgagcaaaggagttgaaggctactacaaatacaaggaagacgtccagttaatggtgcaaacaggactagatgcatacagattctccatttcatggtcaagactaattcccggtggaaaaggacccgtcaacccaaaaggtttacaatattataataactttatcgacgaactcatcaaaaatggaatacaaccgcacgttactctgctgcatttcgacataccggacacacttatgactgcttataatggattgaagggtcaagaatttgtggaagatttcacggcatttgctgacgtgtgcttcaaggaatttggtgaccgagttttgtattggacgacggtcaatgaagcaaataattttgcaagtctaacactcgatgagggcaattttatgccgtctactgaaccgtacattagaggtcacaatatcattcttgctcatgcatccgcggtaaaactataccgagaaaaatataagaaaacccaaaatggattcataggcttgaatttatatgcaagctggtattttcccgagaccgatgacgaacaagattcaattgccgctcaaagagccattgattttactattggatggataatgcaaccattgatatacggagaatatccagaaacattgaagaaacaagtgggagaaagactgccaacatttacaaaagaagagtcaacgttcgttaaaaattcgtttgacttcattggagtgaattgctacgtcggcactgctgttaaggatgaccctgacagctgtaacagtaaaaataaaactattattactgacatgtctgctaaactttctcctaaaggtgaactaggaggagcgtatatgaagggattgttggaatacttcaaaagagattacggcaatccgccaatttacattcaagaaaatggttattggacaccgcgtgaattaggagtgaacgatgcgtcaaggatcgaataccatactgcttctcttgctagcatgcacgatgctatgaagaatggggcaaatgtaaagggatatttccaatggtcatttttggatctcttggaggtgttcaaatacagctatggcctctaccatgtcgatttggaagacccgacccgagaaagacgacccaaggcatccgccaattggtacgcggagttcttgaagggttgcgctacttctaacgggaatgctaaagttgaaactccgttgtaa

SEQ ID NO: 34 - protein, 385 aa, synthetic construct
MVLSRLDFPSDFIFGSGTSASQVEGAALEDGKTSTAFEGFLTRMSGNDLSKGVEGYYKYKEDVQLMVQTGLDAYRFSISWSRLIPGGKGPVNPKGLQYYNNFIDELIKNGIQPHVTLLHFDIPDTLMTAYNGLKGQEFVEDFTAFADVCFKEFGDRVLYWTTVNEANNFASLTLDEGNFMPSTEPYIRGHNIILAHASAVKLYREKYKKTQNGFIGLNLYASWYFPETDDEQDSIAAQRAIDFTIGWIMQPLIYGEYPETLKKQVGERLPTFTKEESTFVKNSFDFIGVNCYVGTAVKDDPDSCNSKNKTIITDMSAKLSPKGELGGAYMKGLLEYFKRDYGNPPIYIQENGYWTPRELGVNDASRIEYHTASLASMHDAMKNGA

SEQ ID NO: 35 - DNA, 1380 bp, synthetic construct
  source 1..1380: mol_type = other DNA
atggaaccttcaaaaatggaagtgaaaataatatcgtccgaaaccatcaaaccgtcatctccgacaccatcccaccttcgaaaatatacactttctttgctcgaccaaaaatacacgcctatcgttgttccggccattctattctatgagcgcccacaaggggtggcgccattggatatggaccgtctcagaacatgcctctcacagacacttaccgcgttttaccctttagccggacgagctgaatctcgagacgttataatatgtaatgacgaaggtatccccttcgttgaggctcatgtcgattgtgaactttcgagtgttgttaagtcgctttcgtccctagggagtgatttgcggtctttttacccgcctagggacggtttactcgaggggggaattcagtttgctattcagatgaatgtgtttagttgtggcgggtttgcgttcgcgtggtattgcacgcataacgttactgacgggacctcgactgctaacttttttaggtattggactgcgctgtatgctcaacgtagtgagtacgcagtccaagacctaatggatttcaattccgtcgtcactgcctttccccctgtgccgccccgtgtaccgcaggaggaaaaaccggtgacaacggaattgaaacccgagaaacaagagggacaagaaaaggaggaaaagaaaaaatcgtcatttaatttcagttttcaatctcacatcgtggcgaggagtttcttgataaagagcaaggcggtcgcagagttgaaggccaagtcggtaagcgaggaagtgccatatccgagtcggttcgaggccgtgtcggctttcctatggaaatcgatagtgtcaagctcgacaacagaagggaagacgatgatcaatatgcccgtaaacttgagaccacgggtggacccgccattacccttggactccgtaggtaacattttcgaaaatgcactcgtacagtccgagaaaaaagcggagctccacgaattcgttgcaaggatccgtggatcaatctcgaaaatgaaagattttgccacggaatatcaaggcgaaaagcgggaagaagctaaggacgcacattggaaaagattcataaaagcggttatcgagtgtaaggggaaagacgcctacgtaatttcgccttggtataagtcgtccgggtttacggacatagatttcgggtttgggaccccgatacgggtcgtacccatggacgatgtcgtaaatcataatcaaaggaacacgataatgttgatggagtttgttgattccgacggtgatggatttgaagcttggatgttcctggaggaggaatgtatcaagtttttggagtccaacccggaatttcttgcctttgcttccccaaacttttaa

SEQ ID NO: 36 - protein, 459 aa, synthetic construct
MEPSKMEVKIISSETIKPSSPTPSHLRKYTLSLLDQKYTPIVVPAILFYERPQGVAPLDMDRLRTCLSQTLTAFYPLAGRAESRDVIICNDEGIPFVEAHVDCELSSVVKSLSSLGSDLRSFYPPRDGLLEGGIQFAIQMNVFSCGGFAFAWYCTHNVTDGTSTANFFRYWTALYAQRSEYAVQDLMDFNSVVTAFPPVPPRVPQEEKPVTTELKPEKQEGQEKEEKKKSSFNFSFQSHIVARSFLIKSKAVAELKAKSVSEEVPYPSRFEAVSAFLWKSIVSSSTTEGKTMINMPVNLRPRVDPPLPLDSVGNIFENALVQSEKKAELHEFVARIRGSISKMKDFATEYQGEKREEAKDAHWKRFIKAVIECKGKDAYVISPWYKSSGFTDIDFGFGTPIRVVPMDDVVNHNQRNTIMLMEFVDSDGDGFEAWMFLEEECIKFLESNPEFLAFASPNF

SEQ ID NO: 37 - DNA, 56 bp, synthetic construct
  source 1..56: mol_type = other DNA
ggggacaagtttgtacaaaaaagcaggcttaatgtggaggttaaaaatagcagaag

SEQ ID NO: 38 - DNA, 54 bp, synthetic construct
  source 1..54: mol_type = other DNA
ggggaccactttgtacaagaaagctgggtattaactaagcctcaaaggaacatg

SEQ ID NO: 39 - DNA, 55 bp, synthetic construct
  source 1..55: mol_type = other DNA
ggggacaagtttgtacaaaaaagcaggcttaatggaactcttcttcatatgtgga

SEQ ID NO: 40 - DNA, 54 bp, synthetic construct
  source 1..54: mol_type = other DNA
ggggaccactttgtacaagaaagctgggtattaagcagatacagttacgggttt

SEQ ID NO: 41 - DNA, 56 bp, synthetic construct
  source 1..56: mol_type = other DNA
ggggacaagtttgtacaaaaaagcaggcttaatggagctaattaccttactaagtg

SEQ ID NO: 42 - DNA, 50 bp, synthetic construct
  source 1..50: mol_type = other DNA
ggggaccactttgtacaagaaagctgggtattaagcgagggtggcggatt

SEQ ID NO: 43 - DNA, 56 bp, synthetic construct
  source 1..56: mol_type = other DNA
ggggacaagtttgtacaaaaaagcaggcttaatgcatagtaatagtaagatgggta

SEQ ID NO: 44 - DNA, 53 bp, synthetic construct
  source 1..53: mol_type = other DNA
ggggaccactttgtacaagaaagctgggtattaacgtctacgaaacatgagag

SEQ ID NO: 45 - DNA, 51 bp, synthetic construct
  source 1..51: mol_type = other DNA
ggggacaagtttgtacaaaaaagcaggcttaatgtcaccccacaacacctg

SEQ ID NO: 46 - DNA, 54 bp, synthetic construct
  source 1..54: mol_type = other DNA
ggggaccactttgtacaagaaagctgggtattaagagcgaccttttctagcttt

SEQ ID NO: 47 - DNA, 55 bp, synthetic construct
  source 1..55: mol_type = other DNA
ggggacaagtttgtacaaaaaagcaggcttaatgggttcaaatacagaagcaact

SEQ ID NO: 48 - DNA, 52 bp, synthetic construct
  source 1..52: mol_type = other DNA
ggggaccactttgtacaagaaagctgggtatcaagccttccttaacgatctc

SEQ ID NO: 49 - DNA, 55 bp, synthetic construct
  source 1..55: mol_type = other DNA
ggggacaagtttgtacaaaaaagcaggcttaatgaagtcaccactaaagttgtac

SEQ ID NO: 50 - DNA, 57 bp, synthetic construct
  source 1..57: mol_type = other DNA
ggggaccactttgtacaagaaagctgggtactaattagcaaccttactcattttatc

SEQ ID NO: 51 - DNA, 57 bp, synthetic construct
  source 1..57: mol_type = other DNA
ggggacaagtttgtacaaaaaagcaggcttaatgtcggatcaaaatgataaaaaggt

SEQ ID NO: 52 - DNA, 56 bp, synthetic construct
  source 1..56: mol_type = other DNA
ggggaccactttgtacaagaaagctgggtattagaaagatgaaacccactcaataa

SEQ ID NO: 53 - DNA, 53 bp, synthetic construct
  source 1..53: mol_type = other DNA
ggggacaagtttgtacaaaaaagcaggcttaatgtctgccaaaatgttgcacg

SEQ ID NO: 54 - DNA, 52 bp, synthetic construct
  source 1..52: mol_type = other DNA
ggggaccactttgtacaagaaagctgggtatcactcgacgagtgcttgtaaa

SEQ ID NO: 55 - DNA, 56 bp, synthetic construct
  source 1..56: mol_type = other DNA
ggggacaagtttgtacaaaaaagcaggcttaatgggtactaaagagttacacatag

SEQ ID NO: 56 - DNA, 54 bp, synthetic construct
  source 1..54: mol_type = other DNA
ggggaccactttgtacaagaaagctgggtactacttctcaacaagatcttgtag

SEQ ID NO: 57 - DNA, 53 bp, synthetic construct
  source 1..53: mol_type = other DNA
ggggacaagtttgtacaaaaaagcaggcttaatggaggaatcaaaggaggaag

SEQ ID NO: 58 - DNA, 55 bp, synthetic construct
  source 1..55: mol_type = other DNA
ggggaccactttgtacaagaaagctgggtatcaaaatttttgtagcacagctttg

SEQ ID NO: 59 - DNA, 55 bp, synthetic construct
  source 1..55: mol_type = other DNA
ggggacaagtttgtacaaaaaagcaggcttaatggaagtgaaaattgtacgtagg

SEQ ID NO: 60 - DNA, 51 bp, synthetic construct
  source 1..51: mol_type = other DNA
ggggaccactttgtacaagaaagctgggtattagctgggcgtggcatattc

SEQ ID NO: 61 - DNA, 25 bp, synthetic construct
  source 1..25: mol_type = other DNA
tcgcgttaacgctagcatggatctc

SEQ ID NO: 62 - DNA, 25 bp, synthetic construct
  source 1..25: mol_type = other DNA
acatcagagattttgagacacgggc

(19) United States
(12) Patent Application Publication
(10) Pub. No.: US 2025/0346914 A1
(43) Pub. Date: Nov. 13, 2025

(54) SAPONARIOSIDE BIOSYNTHETIC ENZYMES

(71) Applicant: PLANT BIOSCIENCE LIMITED, Norwich, Norfolk (GB)

(72) Inventors: Anne OSBOURN, Norwich, Norfolk (GB); Seohyun JO, Norwich, Norfolk (GB)

(21) Appl. No.: 18/871,655

(22) PCT Filed: Jun. 27, 2023
(86) PCT No.: PCT/EP2023/067395

§ 371 Date: Dec. 4, 2024

(30) Foreign Application Priority Data
Jun. 29, 2022 (GB) 2209501.2

(51) Int. Cl. C12N 15/82; C12N 9/10; C12N 9/90

(52) U.S. Cl. CPC C12N 15/8251; C12N 9/1051; C12N 9/90; C12Y 204/01017; C12Y 504/99039

(57) ABSTRACT

This invention relates to methods of producing triterpenoids using one or more of (i) Saponaria officinalis β-amyrin synthase (SobAS) (ii) S. officinalis C28 oxidase (SoC28) (iii) S. officinalis C28C16 oxidase (SoC28C16)(iv) S. officinalis C23 oxidase (SoC23); (v) S. officinalis QA 3-O glucuronosyl transferase SoCSL; (vi) S. officinalis QA-GlcA SoC3Gal; (vii) S. officinalis QA-GlcA-Gal x SoC3Xy; (vii) S. officinalis QA-Tri fucosyl transferase SoC28Fu (ix) S. officinalis QA-TriF rhamnosyl transferase SoC28Rha (x) S. officinalis QA-TriFR xyl SoC28Xul1; (xi) S. officinalis QA-TriFRX xyl SoC28Xyl2; (xii) S. officinalis QA-TriFRXX quinovosyl SoGH1 and (xiii) A. officinalis QA-TroF(Q)RXX acetyl SoBAHD1 polypeptide. Methods, host cells, isolated polypeptides, nucleic acids, and plants are provided.

### FIELD

This invention relates to the biosynthesis of complex triterpenoid saponins and intermediates, such as quillaic acid, and to genes and polypeptides involved in this biosynthesis.

### BACKGROUND

Saponaria officinalis (family Caryophyllaceae), commonly known as soapwort, is a perennial flowering plant native to Europe and Asia that has been used as a traditional source of soap [1]. The well-known detergent property of soapwort is due to the high content of amphiphilic saponins present in the plant extract. The ancient Greeks, Romans and Egyptians used soapwort extracts to clean and wash clothing and later, the first American colonists brought soapwort plants from Europe to North America for their household uses [2]. In addition to their detergent properties, soapwort extracts have been used in folk medicine to treat conditions such as syphilis, gout, rheumatism and jaundice [3]. Soapwort extracts also play an important role in the Middle Eastern culture as the extracts have been used to make tahini halvah, a traditional Middle Eastern dessert [4]. Today, soapwort extracts are still used in cosmetics, nutraceutical and phytomedicinal products [5]. Additionally, saponin layer of soapwort extracts have been investigated for their potential use in bioremediation, as food surfactants, for their anti-fungicidal and immunotoxicity activities [6-9]

S. officinalis is a rich source for saponins with various aglycone cores, such as quillaic acid, gypsogenin and gypsogenic acid. The major saponins found in soapwort extracts are reported as saponariosides A and B (SpA, SpB) [1]. SpA and SpB are similar in chemical structure. They are both composed of quillaic acid aglycone, a C-30 triterpenoid, decorated with a branched trisaccharide at the C-3 position, and a linear tetrasaccharide at the C28 position. The first sugar of the tetrasaccharide chain, β-D-fucose, is linked to a β-D-quinovose with an acetyl group attached. The only chemical difference between SpA and SpB is the addition of a β-D-xylose on the quinovose moiety on the C28 sugar chain in SpA.

Interestingly, QS-21, a triterpenoid saponin found in Quillaja saponaria shares a striking chemical resemblance to SpA and SpB (FIG. 1). QS-21 is a complex triterpenoid saponin synthesised by the Chilean tree Quillaja saponaria (order Fabales). Biochemically, QS-21 consists of a C-30 triterpenoid quillaic acid backbone. This scaffold is decorated with a branched trisaccharide at the C-3 position and a linear tetrasaccharide at the C28 position. The terminal sugar of the tetrasaccharide may be either β-D-apiose or β-D-xylose. Finally, the β-D-fucose sugar within the tetrasaccharide also features a C-18 acyl chain which is glycosylated with an arabinose sugar. QS-21 is a potent immunostimulatory agent capable of enhancing antibody responses and boosting specific T-cell responses, giving it significant adjuvant potential (Del Giudice et al. Seminars in Immunology, 2018. 39: p. 14-21; Marciani, D. J. Trends in Pharmacological Sciences, 2018. 39 (6): p. 573-585). The AS01 adjuvant is a liposomal formulation of QS-21 and 3-O-desacyl-4′-monophosphoryl lipid A and is currently licenced as part of the GlaxoSmithKline ‘Shingrix’ vaccine against herpes zoster and ‘Mosquirix’ against malaria (Del Giudice et al. supra).

Despite the promising commercial potential of saponariosides and their intermediates, nothing is known of their biosynthetic pathway. The biosynthesis of saponariosides can be conceptually divided into two stages: (i) the biosynthesis of the quillaic acid core and (ii) the decoration of quillaic acid (FIG. 2). However, the actual order can be different in planta and further details are unknown. Many plant natural products are present in low abundance in the plant, and chemical synthesis is often non-viable due to the complex chemical structures. Knowledge of the biosynthetic pathway may allow for metabolic engineering in alternative host system, allowing for large-scale production of the compound of interest.

### SUMMARY

The present inventors have identified and characterised the genes involved in the biosynthesis of complex triterpenoid saponins in the soapwort plant (Saponaria officinalis). These include genes encode enzymes involved in the biosynthesis of QA and glycosyl transferases involved in the glycosylation of QA. Expression of one or more of these genes may be useful in the production of QA and glycosylation products of QA.

A first aspect of the invention provides a method for the production of a triterpenoid comprising one or more of;
(i) contacting 2,3-oxidosqualene (OS) with a Saponaria officinalis β-amyrin synthase (SobAS) comprising an amino acid sequence having at least 80% sequence identity to SEQ ID NO 8, such that said OS is converted into β-amyrin;
(ii) either;
   (a) contacting β-amyrin with a SoC28 oxidase polypeptide comprising an amino acid sequence having at least 80% sequence identity to SEQ ID NO 2, such that the C28 position of said β-amyrin is oxidised to a carboxylic acid to produce oleanolic acid and contacting oleanolic acid with a SoC28C16 oxidase polypeptide comprising an amino acid sequence having at least 50% sequence identity to SEQ ID NO 4, such that the C16 position of said oleanolic acid is oxidised to an alcohol, thereby producing echinocystic acid; or
   (b) contacting β-amyrin with a SoC28C16 oxidase polypeptide comprising an amino acid sequence having at least 50% sequence identity to SEQ ID NO 4, such that the C28 position of said β-amyrin is oxidised to a carboxylic acid and the C16 position of said β-amyrin is oxidised to an alcohol, thereby producing echinocystic acid;
(iii) contacting echinocystic acid with a SoC23 oxidase polypeptide comprising an amino acid sequence having at least 50% sequence identity to SEQ ID NO 6, such that the C-23 position of said echinocystic acid is oxidised to an aldehyde, thereby producing quillaic acid (QA);
(iv) contacting QA with Saponaria officinalis QA 3-O glucuronosyl transferase (“SoCSL”) polypeptide comprising an amino acid sequence having at least 60% sequence identity to SEQ ID NO: 10, such that said QA is converted into QA-GlcA;
(v) contacting QA-GlcA with a Saponaria officinalis QA-GlcA galactosyl transferase (“SoC3Gal”) polypeptide comprising an amino acid sequence having at least 50% sequence identity to SEQ ID NO: 12, such that said QA-GlcA is converted into QA-GlcA-Gal;
(vi) contacting QA-GlcA-Gal with a Saponaria officinalis QA-GlcA-Gal xylosyl transferase (“SoC3Xyl”) polypeptide comprising an amino acid sequence having at least 50% sequence identity to SEQ ID NO: 14, such that said QA-GlcA-Gal is converted into QA-Tri (QA-GlcA-Gal-Xyl);
(vii) contacting QA-Tri with a Saponaria officinalis QA-Tri fucosyl transferase (“SoC28Fu”) polypeptide comprising an amino acid sequence having at least 60% sequence identity to SEQ ID NO: 16, such that said QA-Tri is converted into QA-TriF;
(viii) contacting QA-TriF with a Saponaria officinalis QA-TriF rhamnosyl transferase (“SoC28Rha”) polypeptide comprising an amino acid sequence having at least 50% sequence identity to SEQ ID NO: 18, such that said QA-TriF is converted into QA-TriFR;
(ix) contacting QA-TriFR with a Saponaria officinalis QA-TriFR xylosyl transferase (“SoC28Xyl1”) polypeptide comprising an amino acid sequence having at least 50% sequence identity to SEQ ID NO: 20, such that said QA-TriFR is converted into QA-TriFRX; and/or
(x) contacting QA-TriFRX with a Saponaria officinalis QA-TriFRX xylosyl transferase (“SoC28Xyl2”) polypeptide comprising an amino acid sequence having at least 50% sequence identity to SEQ ID NO: 22, such that said QA-TriFRX is converted into QA-TriFRXX,
(xi) contacting QA-TriFRXX with a Saponaria officinalis QA-TriFRXX quinovosyl transferase (“SoGH1”) polypeptide comprising an amino acid sequence having at least 50% sequence identity to SEQ ID NO: 34, such that said QA-TriFRXX is converted into QA-TriF(Q)RXX, and/or
(xii) contacting QA-TriF(Q)RXX with a Saponaria officinalis QA-TriF(Q)RXX acetyl transferase (“SoBAHD1”) polypeptide comprising an amino acid sequence having at least 50% sequence identity to SEQ ID NO: 36, such that said QA-TriF(Q)RXX is converted into saponarioside B (SpB).

A method of the first aspect may comprise;
contacting β-amyrin with a Saponaria officinalis C28 oxidase (SoC28 oxidase) to oxidise the C28 position of the β-amyrin to a carboxylic acid to form oleanolic acid,
wherein the amino acid sequence of the SoC28 oxidase has at least 80% sequence identity to SEQ ID NO: 2;
contacting the oleanolic acid with a Saponaria officinalis C28C16 oxidase (SoC28C16 oxidase) to oxidise the C16 position of the oleanolic acid to an alcohol to form echinocystic acid,
wherein the amino acid sequence of the SoC28C16 oxidase has at least 50% sequence identity to SEQ ID NO: 4; and
contacting the echinocystic acid with a Saponaria officinalis C-23 oxidase (SoC23 oxidase) to oxidise the C16 position of echinocystic acid to an aldehyde to form quillaic acid (QA),
wherein the amino acid sequence of the SoC23 oxidase having at least 50% sequence identity to SEQ ID NO: 6.

A method of the first aspect may comprise;
contacting β-amyrin with a Saponaria officinalis C28C16 oxidase (SoC28C16 oxidase) to oxidise the C28 position of the β-amyrin to a carboxylic acid and the C16 position to an alcohol to form echinocystic acid, wherein the amino acid sequence of the SoC2816 oxidase has at least 50% sequence identity to SEQ ID NO: 4; and
contacting the echinocystic acid with a Saponaria officinalis C-23 oxidase (SoC23 oxidase) to oxidise the C16 position of echinocystic acid to an aldehyde to form quillaic acid (QA), wherein the amino acid sequence of the SoC23 oxidase having at least 50% sequence identity to SEQ ID NO: 6.

A method of the first aspect may comprise or further comprise;
contacting QA with a Saponaria officinalis QA 3-O glucuronosyl transferase (“SoCSL”) to covalent attach D-glucuronic acid (“GlcA”) to the 3-O position of quillaic acid to form 3-O-{β-D-glucopyranosiduronic acid}-quillaic acid (“QA-GlcA” or “QA-Mono”);
wherein the amino acid sequence of the SoCSL has at least 60% sequence identity to SEQ ID NO: 10;
contacting QA-GlcA with Saponaria officinalis QA-GlcA galactosyl transferase (“SoC3Gal”) to covalently attach D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-GlcA-Gal or QA-Di”);
wherein the amino acid sequence of the SoC3Ga has at least 50% sequence identity to SEQ ID NO: 12; and
contacting QA-GlcA-Gal with a Saponaria officinalis QA-GlcA-Gal xylosyl transferase (“SoC3Xyl”) to covalently attach D-Xylose (“Xyl”) via a 1,3 linkage to QA-GlcA-Gal to form 3-O-{β-D-xylopyranosyl-(1->3)-[β-D-galactopyranosyl-(1->2)]-β-D-glucopyranosiduronic acid}-quillaic acid (“QA-Tri” or “QA-GlcA-Gal-Xyl”);
wherein the amino acid sequence of SoC3Xyl has at least 50% sequence identity to SEQ ID NO: 14.

A method of the first aspect may comprise or further comprise;
contacting 3-O-{β-D-xylopyranosyl-(1->3)-[β-D-galactopyranosyl-(1->2)]-β-D-glucopyranosiduronic acid}-quillaic acid (QA-Tri) with a Saponaria officinalis QA-Tri fucosyl transferase (“SoC28Fu”) to attach fucose to the 28-O position of QA-Tri to form 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF)
wherein the amino acid sequence of SoC28Fu has at least 60% sequence identity to SEQ ID NO: 16;
contacting QA-TriF with a Saponaria officinalis QA-TriF rhamnosyl transferase (“SoC28Rha”) to covalently attach rhamnose via a 1, 2 linkage to QA-TriF to form 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR);
wherein the amino acid sequence of SoC28Rha has at least 50% sequence identity to SEQ ID NO: 18;
contacting QA-TriFR with a Saponaria officinalis QA-TriFR xylosyl transferase (“SoC28Xyl1”) to covalently attach xylose via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX), wherein the amino acid sequence of SoC28Xyl1 has at least 50% sequence identity to SEQ ID NO: 20;
contacting QA-TriFRX with a Saponaria officinalis QA-TriFRX-xylosyl transferase (“SoC28Xyl2”) to covalently attach xylose via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX, wherein the amino acid sequence of SoC28Xyl2 has at least 50% sequence identity to SEQ ID NO: 22.

A method of the first aspect may comprise or further comprise;
contacting QA-TriFRXX with a Saponaria officinalis QA-TriFRXX quinovosyl transferase (“SoGH1”) to covalently attach quinovose via a 1,4 linkage to QA-TriFRXX to form 3-O-{β-D-xylopyranosyl-(1→3)-[β-D-galactopyranosyl-(1 →2)]-β-D-glucopyranosiduronic acid}-28-O-{β-D-xylopyranosyl-(1→3)-3-D-xylopyranosyl-(1→4)-α-I-rhamnopyranosyl-(1→2)-[β-d-quinovopyranosyl-(1→4)]-β-d-fucopyranosyl ester}-quillaic acid (QA-TriF(Q)RXX), wherein the amino acid sequence of SoGH1 has at least 50% sequence identity to SEQ ID NO: 34; and/or
contacting QA-TriF(Q)RXX with a Saponaria officinalis QA-TriF(Q)RXX acetyl transferase (“SoBAHD1”) to covalently attach an acetyl group to QA-TriF(Q)RXX to form saponarioside B, wherein the amino acid sequence of SoBAHD1 has at least 50% sequence identity to SEQ ID NO: 36.

A second aspect of the invention provides method of converting a host from a phenotype whereby the host is unable to carry out triterpenoid biosynthesis from β-amyrin to a phenotype whereby the host is able to carry out said triterpenoid biosynthesis, the method comprising;
expressing a heterologous nucleic acid within the host or one or more cells thereof, following an earlier step of introducing the nucleic acid into the host or an ancestor of either,
wherein the heterologous nucleic acid encodes one or more or all of the following polypeptides
(i) a SoC28 oxidase capable of oxidising β-amyrin at the C28 position to a carboxylic acid (“SoC28 oxidase”); said SoC28 oxidase having at least 80% sequence identity to SEQ ID NO:2;
(ii) a SoC28C16 oxidase capable of oxidising β-amyrin at the C28 position to a carboxylic acid and at the C16 position to an alcohol (“SoC28C16 oxidase”); said C28C16 oxidase having at least 50% sequence identity to SEQ ID NO: 4; and
(iii) a SoC23 oxidase capable of oxidising echinocystic acid at the C-23 position to an aldehyde (“SoC23 oxidase”), SoC23 oxidase having at least 50% sequence identity to SEQ ID NO: 6,
(iv) a Saponaria officinalis QA 3-O glucuronosyl transferase (“SoCSL”) capable of attaching D-glucuronic acid (“GlcA”) to the 3-O position of quillaic acid to form 3-O-{β-D-glucopyranosiduronic acid}-quillaic acid (“QA-Mono” or “QA-GlcA”); said SoCSL having at least 60% sequence identity to SEQ ID NO: 10;
(v) Saponaria officinalis QA-GlcA galactosyl transferase (“SoC3Gal”) capable of attaching D-Galactose (“Gal”) via a β-1->2 linkage to QA-GlcA to form 3-O-{[β-D-galactopyranosyl-(1->2)]-3-D-glucopyranosiduronic acid}-quillaic acid (“QA-Di” or “QA-GlcA-Gal”); wherein the amino acid sequence of the SoC3Gal has at least 50% sequence identity to SEQ ID NO: 12;
(vi) a Saponaria officinalis QA-GlcA-Gal xylosyl transferase (“SoC3Xyl”) capable of attaching D-Xylose (“Xyl”) via a 1,3 linkage to QA-GlcA-Gal to form 1, 3-O-{β-D-xylopyranosyl-(1->3)-[β-D-galactopyranosyl-(1->2)]-β-D-glucopyranosiduronic acid}-quillaic acid (“QA-GlcA-Gal-Xyl” or “QA-Tri”), wherein the amino acid sequence of SoC3Xyl has at least 50% sequence identity to SEQ ID NO: 14;
(vii) a Saponaria officinalis QA-Tri fucosyl transferase (“SoC28Fu”) capable of attaching fucose (“Fuc”) to the 28-O position of QA-Tri to form 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF), wherein the amino acid sequence of SoC28Fu has at least 50% sequence identity to SEQ ID NO: 16;
(viii) a Saponaria officinalis QA-TriF rhamnosyl transferase (“SoC28Rha”) capable of attaching rhamnose (“Rha”) via a 1, 2 linkage to QA-TriF to form 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR); said SoC28Rha having at least 50% sequence identity to SEQ ID NO: 18;
(ix) Saponaria officinalis QA-TriFR xylosyl transferase (“SoC28Xyl1”) capable of attaching D-Xylose (“Xyl”) via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX); wherein the amino acid sequence of the C28Xyl1 has at least 50% sequence identity to SEQ ID NO: 20;
(x) a Saponaria officinalis QA-TriFRX xylosyl transferase (“C28Xyl2”) for attachment of D-Xylose (“Xyl”) via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX), wherein the amino acid sequence of C28Xyl2 has at least 50% sequence identity to SEQ ID NO: 22,
(xi) a Saponaria officinalis QA-TriFRXX quinovosyl transferase (“SoGH1”) for attachment of quinovose via a 1, 4 linkage to QA-TrFRXX to form 3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[3-dD-quinovopyranosyl-(1→4)]-3-dD-fucopyranosyl ester}-quillaic acid (QA-TriF(Q)RXX), wherein the amino acid sequence of SoGH1 has at least 50% sequence identity to SEQ ID NO: 34, and/or
(xii) a Saponaria officinalis QA-TriF(Q)RXX acetyl transferase (“SoBAHD1”) for attachment of an acetyl group to QA-TriF(Q)RXX to form saponarioside B, wherein the amino acid sequence of SoBAHD1 has at least 50% sequence identity to SEQ ID NO: 36.

The heterologous nucleic acid in methods of the second aspect may encode the following polypeptides;
(i) a SoC28 oxidase capable of oxidising β-amyrin at the C28 position to a carboxylic acid to form oleanolic acid; said SoC28 oxidase having at least 80% sequence identity to SEQ ID NO: 2;
(ii) a SoC28C16 oxidase capable of oxidising β-amyrin at the C28 position to a carboxylic acid and the C16 position to an alcohol (“C28C16 oxidase”) to form echinocystic acid; said SoC28C16 oxidase having at least 50% sequence identity to SEQ ID NO: 4; and
(iii) a SoC23 oxidase capable of oxidising echinocystic acid at the C-23 position to an aldehyde to form quillaic acid (QA), said SoC23 oxidase having at least 50% sequence identity to SEQ ID NO: 6.

The heterologous nucleic acid in methods of the second aspect may further encode the following polypeptides;
(iv) a Saponaria officinalis QA 3-O glucuronosyl transferase (“SoCSL”) for attachment of D-glucuronic acid (“GlcA”) to the 3-O position of quillaic acid to form 3-O-{β-D-glucopyranosiduronic acid}-quillaic acid (“QA-GlcA”); wherein the amino acid sequence of the SoCSL has at least 60% sequence identity to SEQ ID NO: 10;
(v) Saponaria officinalis QA-GlcA galactosyl transferase (“SoC3Gal”) for attachment D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-GlcA-Gal”); wherein the amino acid sequence of the SoC3Gal has at least 50% sequence identity to SEQ ID NO: 12; and
(vi) a Saponaria officinalis QA-GlcA-Gal xylosyl transferase (“SoC3Xyl”) for attachment of D-Xylose (“Xyl”) via a 1,3 linkage to QA-GlcA-Gal to form 3-O-{β-D-xylopyranosyl-(1->3)-[3-D-galactopyranosyl-(1->2)]-β-D-glucopyranosiduronic acid}-quillaic acid (“QA-GlcA-Gal-Xyl” or “QA-Tri”), wherein the amino acid sequence of SoC3Xyl has at least 50% sequence identity to SEQ ID NO: 14.

The heterologous nucleic acid in methods of the second aspect may further encode the following polypeptides;
(vii) a Saponaria officinalis QA-Tri fucosyl transferase (“SoC28Fu”) for the attachment of fucose(“Fuc”) to the 28-O position of QA-Tri to form 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF); said SoC28Fu having at least 60% sequence identity to SEQ ID NO: 16;
(viii) a Saponaria officinalis QA-TriF rhamnosyl transferase (“SoC28Rha”) for the attachment of rhamnose (“Rha”) via a 1, 2 linkage to QA-TriF to form 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR); said SoC28Rha having at least 50% sequence identity to SEQ ID NO: 18;
(ix) Saponaria officinalis QA-TriFR xylosyl transferase (“SoC28Xyl1”) for attachment of D-Xylose (“Xyl”) via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX); wherein the amino acid sequence of the SoC28Xyl1 has at least 50% sequence identity to SEQ ID NO: 20; and
(x) a Saponaria officinalis QA-TriFRX xylosyl transferase (“SoC28Xyl2”) for attachment of D-Xylose (“Xyl”) via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX); wherein the amino acid sequence of SoC28Xyl2 has at least 80% sequence identity to SEQ ID NO:22.

The heterologous nucleic acid in methods of the second aspect may further encode the following polypeptides;
(xi) a Saponaria officinalis QA-TriFRXX quinovosyl transferase (“SoGH1”) for attachment of quinovose (Q) via a 1, 4 linkage to QA-TrFRXX to form 3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[β-D-quinovopyranosyl-(1→4)]-β-D-fucopyranosyl ester}-quillaic acid (QA-TriF(Q)RXX), wherein the amino acid sequence of SoGH1 has at least 50% sequence identity to SEQ ID NO: 34, and/or
(xii) a Saponaria officinalis QA-TriF(Q)RXX acetyl transferase (“SoBAHD1”) for attachment of an acetyl group to QA-TriF(Q)RXX to form saponarioside B, wherein the amino acid sequence of SoBAHD1 has at least 50% sequence identity to SEQ ID NO: 36.

A third aspect of the invention provides host cell containing or transformed with a heterologous nucleic acid which comprises a plurality of nucleotide sequences, each of which encodes a polypeptide which in combination have triterpenoid biosynthesis activity,
wherein the plurality of nucleotide sequences encode one or more of following polypeptides
(i) a SoC28 oxidase capable of oxidising β-amyrin at the C28 position to a carboxylic acid (“SoC28oxidase”); said SoC28 oxidase having at least 80% sequence identity to SEQ ID NO: 2;
(ii) a SoC28C16 oxidase capable of oxidising β-amyrin at the C28 position to a carboxylic acid and the C16 position to an alcohol (“C28C16 oxidase”); said C28C16 oxidase having at least 50% sequence identity to SEQ ID NO: 4; and
(iii) a SoC23 oxidase capable of oxidising echinocystic acid at the C-23 position to an aldehyde (“SoC23 oxidase”), said SoC23 oxidase having at least 50% sequence identity to SEQ ID NO: 6;
(iv) a Saponaria officinalis QA 3-O glucuronosyl transferase (“SoCSL”) for attachment of D-glucuronic acid (“GlcA”) to the 3-O position of quillaic acid to form 3-O-{β-D-glucopyranosiduronic acid}-quillaic acid (“QA-GlcA”); said SoQA-GlcT having at least 60% sequence identity to SEQ ID NO: 10;
(v) Saponaria officinalis QA-GlcA galactosyl transferase (“SoC3Gal”) for attachment D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-GlcA-Gal”); wherein the amino acid sequence of the SoC3Gal has at least 50% sequence identity to SEQ ID NO: 12; and
(vi) a Saponaria officinalis QA-GlcA-Gal xylosyl transferase (“SoC3Xyl”) for attachment of D-Xylose (“Xyl”) via a 1,3 linkage to QA-GlcA-Gal to form 3-O-{β-D-xylopyranosyl-(1->3)-[3-D-galactopyranosyl-(1->2)]-β-D-glucopyranosiduronic acid}-quillaic acid (“QA-GlcA-Gal-Xyl” or “QA-Tri”), wherein the amino acid sequence of SoC3Xyl has at least 50% sequence identity to SEQ ID NO: 14;
(vii) a Saponaria officinalis QA-Tri fucosyl transferase (“SoC28Fu”) for the attachment of fucose(“Fuc”) to the 28-O position of QA-Tri to form 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF); said SoC28Fu having at least 50% sequence identity to SEQ ID NO: 16;
(viii) a Saponaria officinalis QA-TriF rhamnosyl transferase (“SoC28Rha”) for the attachment of rhamnose (“Rha”) via a 1, 2 linkage to QA-TriF to form 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR); said SoC28Rha having at least 50% sequence identity to SEQ ID NO: 18;
(ix) Saponaria officinalis QA-TriFR xylosyl transferase (“SoC28Xyl1”) for attachment of D-Xylose (“Xyl”) via a 1,4 linkage to QA-TriFR to form 3-O-{β-D-xylopyranosyl-(1->3)-[3-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX)”); wherein the amino acid sequence of the SoC28Xyl1 has at least 50% sequence identity to SEQ ID NO: 20;
(x) a Saponaria officinalis QA-TriFRX xylosyl transferase (“SoC28Xyl2”) for attachment of D-Xylose (“Xyl”) via a 1,3 linkage to QA-TriFRX to form 3-O-{β-D-xylopyranosyl-(1->3)-[3-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX), wherein the amino acid sequence of SoC28Xyl2 has at least 50% sequence identity to SEQ ID NO: 22
(xi) a Saponaria officinalis QA-TriFRXX quinovosyl transferase (“SoGH1”) for attachment of quinovose (Q) via a 1, 4 linkage to QA-TrFRXX to form 3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[β-D-quinovopyranosyl-(1→4)]-3-D-fucopyranosyl ester}-quillaic acid (QA-TriF(Q)RXX), wherein the amino acid sequence of SoGH1 has at least 50% sequence identity to SEQ ID NO: 34, and/or
(xii) a Saponaria officinalis QA-TriF(Q)RXX acetyl transferase (“SoBAHD1”) for attachment of an acetyl group to QA-TriF(Q)RXX to form saponarioside B, wherein the amino acid sequence of SoBAHD1 has at least 50% sequence identity to SEQ ID NO: 36.

The plurality of nucleotide sequences in host cells of the third aspect may encode the following polypeptides;
(i) a SoC28 oxidase capable of oxidising β-amyrin thereof at the C28 position to a carboxylic acid to form oleanolic acid; said SoC28 oxidase having at least 80% sequence identity to SEQ ID NO: 2;
(ii) a SoC28C16 oxidase capable of oxidising β-amyrin at the C28 position to a carboxylic acid and/or the C16 position to an alcohol (“C28C16 oxidase”); and
(iii) a SoC23 oxidase capable of oxidising echinocystic acid at the C-23 position to an aldehyde to form quillaic acid (QA), said SoC23 oxidase having at least 50% sequence identity to SEQ ID NO: 6.

The plurality of nucleotide sequences in host cells of the third aspect may further encode the following polypeptides;
(iv) a Saponaria officinalis QA 3-O glucuronosyl transferase (“SoCSL”) for attachment of D-glucuronic acid (“GlcA”) to the 3-O position of quillaic acid to form 3-O-{β-D-glucopyranosiduronic acid}-quillaic acid (“QA-GlcA”); said SoQA-GlcT having at least 60% sequence identity to SEQ ID NO: 10;
(v) Saponaria officinalis QA-GlcA galactosyl transferase (“SoC3Gal”) for attachment D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-GlcA-Gal”); wherein the amino acid sequence of the QA-GlcA-Gal has at least 50% sequence identity to SEQ ID NO: 12; and
(vi) a Saponaria officinalis QA-GlcA-Gal xylosyl transferase (“SoC3Xyl”) for attachment of D-Xylose (“Xyl”) via a 1,3 linkage to QA-GlcA-Gal to form 3-O-{β-D-xylopyranosyl-(1->3)-[3-D-galactopyranosyl-(1->2)]-β-D-glucopyranosiduronic acid}-quillaic acid (“QA-GlcA-Gal-Xyl” QA-Tri), wherein the amino acid sequence of SoC3Xyl has at least 50% sequence identity to SEQ ID NO: 14.

The plurality of nucleotide sequences in host cells of the third aspect may further encode the following polypeptides;
(vii) a Saponaria officinalis QA-Tri fucosyl transferase (“SoC28Fu”) for the attachment of fucose(“Fuc”) to the 28-O position of QA-Tri to form 3-O-{β-D-xylopyranosyl-(1->3)-[3-D-galactopyranosyl-(1->2)]-β-D-glucopyranosiduronic acid}-28-O-{β-D-fucopyranosyl ester}-quillaic acid (QA-TriF); said SoC28Fu having at least 60% sequence identity to SEQ ID NO: 16;
(viii) a Saponaria officinalis QA-TriF rhamnosyl transferase (“SoC28Rha”) for the attachment of rhamnose (“Rha”) via a 1, 2 linkage to QA-TriF to form 3-O-{β-D-xylopyranosyl-(1->3)-[β-D-galactopyranosyl-(1->2)]-3-D-glucopyranosiduronic acid}-28-O-{α-L-rhamnopyranosyl-(1->2)-β-D-fucopyranosyl ester}-quillaic acid (QA-TriFR); said SoC28Rha having at least 50% sequence identity to SEQ ID NO: 18;
(ix) Saponaria officinalis QA-TriFR xylosyl transferase (“SoC28Xyl1”) for attachment of D-Xylose (“Xyl”) via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX); wherein the amino acid sequence of the SoC28Xyl1 has at least 50% sequence identity to SEQ ID NO: 20; and
(x) a Saponaria officinalis QA-TriFRX xylosyl transferase (“SoC28Xyl2”) for attachment of D-Xylose (“Xyl”) via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX); wherein the amino acid sequence of SoC28Xyl2 has at least 80% sequence identity to SEQ ID NO:22.

The plurality of nucleotide sequences in host cells of the third aspect may further encode the following polypeptides;

(xi) a Saponaria officinalis QA-TriFRXX quinovosyl transferase (“SoGH1”) for attachment of quinovose (Q) via a 1, 4 linkage to QA-TrFRXX to form 3-O-{β-d-xylopyranosyl-(1→3)-[β-d-galactopyranosyl-(1→2)]-β-d-glucopyranosiduronic acid}-28-O-{β-d-xylopyranosyl-(1→3)-β-d-xylopyranosyl-(1→4)-α-I-rhamnopyranosyl-(1→2)-[β-d-quinovopyranosyl-(1→4)]-β-d-fucopyranosyl ester}-quillaic acid (QA-TriF(Q)RXX), wherein the amino acid sequence of SoGH1 has at least 50% sequence identity to SEQ ID NO: 34, and/or
(xii) a Saponaria officinalis QA-TriF(Q)RXX acetyl transferase (“SoBAHD1”) for attachment of an acetyl group to QA-TriF(Q)RXX to form saponarioside B, wherein the amino acid sequence of SoBAHD1 has at least 50% sequence identity to SEQ ID NO: 36.

A fourth aspect of the invention provides a method of producing a host cell comprising transforming or transfecting a host cell with a heterologous nucleic acid which comprises a plurality of nucleotide sequences as set out in the second and third aspects.

A fifth aspect provides a process for producing a transgenic plant which method comprises the steps of:
(a) performing a method of the fourth aspect, wherein the host cell is a plant cell, and
(b) regenerating a plant from the transformed plant cell.

A sixth aspect provides a transgenic plant which is obtainable by the method of the fifth aspect, or which is a clone, or selfed or hybrid progeny or other descendant of said transgenic plant, wherein expression of said heterologous nucleic acid imparts an increased ability to carry out the biosynthesis compared to a wild-type plant otherwise corresponding to said transgenic plant.

A seventh aspect provides a method of producing a triterpenoid in a heterologous host, which method comprises culturing a host cell as set out in the third aspect and purifying the triterpenoid therefrom.

An eighth aspect provides a method of producing a triterpenoid in a heterologous host, which method comprises growing a plant of the sixth aspect and then harvesting it and purifying the triterpenoid therefrom.

A triterpenoid of the seventh and eighth aspects may be QA or a glycosylated QA, such as QA-Tri, QA-TriFRXX or QA-F(Q-Ac)RXX or an intermediate or derivative thereof

The SobAS, SoC28 oxidase, SoC23 oxidase, SoC28C16 oxidase, SoCSL, SoC3Gal, SoC3Xyl, SoC28Fu, SoC28Rha, SoC28Xyl1, SoC28Xyl2, SoGH1 and SoBAHD1 of the first to the eighth aspects may be obtained or derived from Saponaria officinalis.

Other aspects and embodiments of the invention are described in more detail below.

### BRIEF DESCRIPTION OF THE FIGURES

FIG. 1 shows the chemical structure of (A) saponariosides A/B from S. officinalis and (B) QS-21 from Q. saponaria.

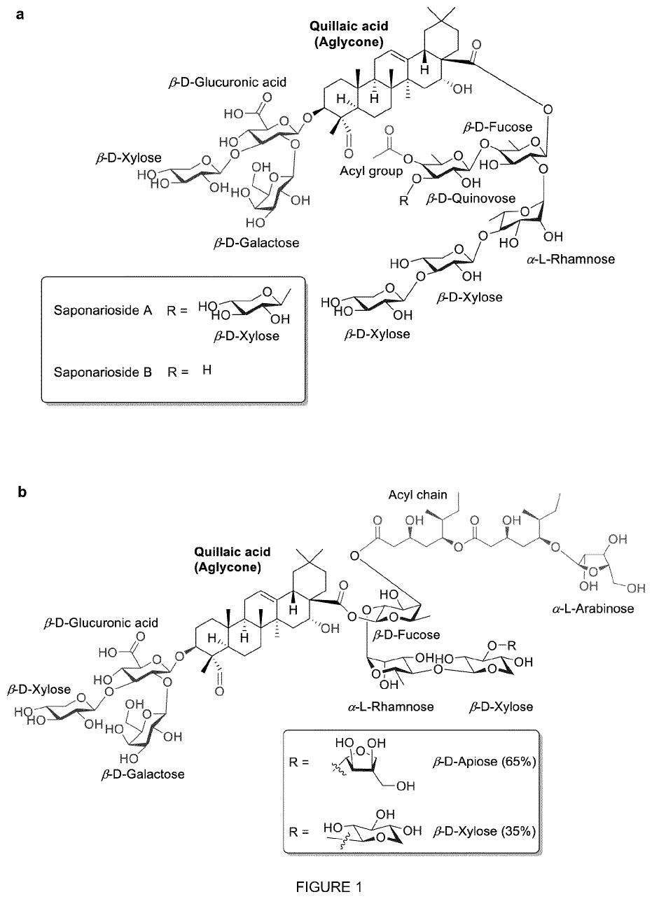

FIG. 2 shows the predicted biosynthetic pathway of saponariosides A/B. (A) Biosynthesis of the aglycone quillaic acid from 2,3-oxidosqualene. (B) Predicted order of quillaic acid decoration. QA, quillaic acid; F, fucose; Rh, rhamnose; X, xylose; Q, quinovose; A, acetyl moiety; GA, glucuronic acid; Gal, galactose; Ar, arabinose. (C) Simplified chemical structures of saponarioside A and B. GlcA, glucuronic acid; Xyl, xylose; Gal, galactose; Fuc, fucose; Rha, rhamnose; Qui, quinovose; Acyl, acetyl moiety.

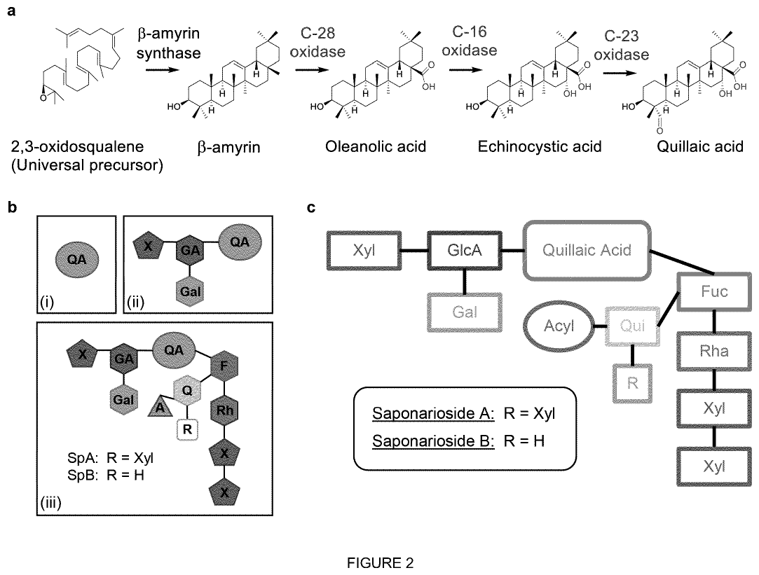

FIG. 3 shows the expression profile of candidate soapwort genes across different soapwort organs. Pearson's correlation coefficient (PCC) to SobAS1 was used to identify candidate genes of interest. The heatmap shows the raw RNA-Seq read counts normalized to the library size and rlog-transformed. The functional soapwort genes are labelled in bold and absolute transcript read counts of candidate genes are also shown.

FIG. 4 shows the characterization of SobAS. N. benthamiana leaves transiently expressing AstHMGR and SobAS were extracted and analysed using GC/MS. (A) The total ion chromatograms (TIC) and the mass spectra are shown. The extract from N. benthamiana leaves expressing only AstHMGR was used as a negative control. β-amyrin peak was identified by comparison with commercial standard. Peak 1, β-amyrin. (B) The function of SobAS1.

FIG. 5 shows the characterization of SoCYP716A378, SoCYP716A379 and SoCYP72A984. (A) Pathway depicting the functions of SobAS1, SoCYP716A378, SoCYP716A379 and SoCYP72A984 in converting oxidosqualene to Quillaic acid (4). N. benthamiana leaves transiently expressing various combinations of CYPs genes were extracted and analysed using GC-MS or HPLC-MS. The extract from N. benthamiana leaves expressing only AstHMGR was used as a negative control (control) and highlighted peaks were identified by comparison with commercial standards (β-amyrin (1), oleanolic acid (2), echinocystic acid (3) and quillaic acid (4)). (B) GC-MS total ion chromatograms (TIC) and relevant mass spectra for N. benthamiana leaves transiently co-expressing tHMGR and SobAS1 with either SoCYP716A378 or SoCYP716A379. The activity of SoCYP716A378 produced a single peak of (2) while the activity of SoCYP716A379 produced an additional peak of (3). (C) HPLC-MS extracted ion chromatograms (EIC) and relevant mass spectra for N. benthamiana leaves transiently co-expressing tHMGR, SobAS1 and SoCYP716A379, with and without SoCYP72A984. EIC displayed are for m/z 485.3267, the calculated mass of [M-H]⁻ adduct of quillaic acid (4). The additional activity of SoCYP72A984 produced a major new peak corresponding to (4)

FIG. 6 shows the characterization of SoCSL1. (A) Structure of 3-O-{β-D-glucopyranosiduronic acid}-quillaic acid (QA-Mono, (5)), the product of SoCSL1 when acting in combination with the S. officinalis enzymes required for production of quillaic acid (QA, (4)). Modification performed by SoCSL1 has been highlighted and a table showing relevant calculated adducts and fragments of (5) included. (B) N. benthamiana leaves transiently co-expressing various genes were extracted and analysed using HPLC-MS, representative extracted ion chromatograms (EIC) and MS/MS spectra are shown. EIC displayed are for m/z 661.3588, the calculated mass of the [M-H]⁻ adduct of (5). The negative controls used were extracts from N. benthamiana leaves co-expressing only AstHMGR (tHMGR control) or co-expressing the S. officinalis genes required to produce (4) (tHMGR, SobAS1, SoCYP716A379 and SoCYP72A984) (QA). The additional activity of SoCSL1 produced a peak corresponding to (5), identified by comparison with an authentic standard.

FIG. 7 shows the characterization of SoUGT73DL1. (A) Structure of 3-O-{[β-D-galactopyranosyl-(1->2)]-β-D-glucopyranosiduronic acid}-quillaic acid (QA-Di, (6)), the product of SoUGT73DL1 when acting in combination with the S. officinalis enzymes required for production of 3-O-{β-D-glucopyranosiduronic acid}-quillaic acid (QA-mono, (5)). Modification performed by SoUGT73DL1 has been highlighted and a table showing relevant calculated adducts and fragments of (6) included. (B) N. benthamiana leaves transiently co-expressing various genes were extracted and analysed using HPLC-MS, representative (n=6) extracted ion chromatograms (EIC) and MS/MS spectra are shown. EIC displayed are for m/z 823.4116, the calculated mass of the [M-H]⁻ adduct of (6). The negative controls used were extracts from N. benthamiana leaves co-expressing only AstHMGR (tHMGR control) or co-expressing the S. officinalis genes required to produce (5) (tHMGR, SobAS1, SoCYP716A379, SoCYP72A984 and SoCSL1) (QA-mono). The additional activity of SoUGT73DL1 produced a peak corresponding to (6), identified by comparison with an authentic standard.

FIG. 8 shows the characterization of SoUGT73CC6. (A) Structure of 3-O-{β-D-xylopyranosyl-(1->3)-[β-D-galactopyranosyl-(1->2)]-β-D-glucopyranosiduronic acid}-quillaic acid (QA-Tri, (7)), the product of SoUGT73CC6 when acting in combination with the S. officinalis enzymes required for production of 3-0-{[β-D-galactopyranosyl-(1->2)]-3-D-glucopyranosiduronic acid}-quillaic acid (QA-Di, (6)). Modification performed by SoUGT73CC6 has been highlighted and a table showing relevant calculated adducts and fragments of (7) included. (B) N. benthamiana leaves transiently co-expressing various genes were extracted and analysed using HPLC-MS, representative (n=6) extracted ion chromatograms (EIC) and MS/MS spectra are shown. EIC displayed are for m/z 955.4539, the calculated mass of the [M-H]⁻ adduct of (7). The negative controls used were extracts from N. benthamiana leaves co-expressing only AstHMGR (tHMGR control) or co-expressing the S. officinalis genes required to produce (6) (tHMGR, SobAS1, SoCYP716A379, SoCYP72A984, SoCSL and SoUGT73DL1) (QA-Di). The additional activity of SoUGT73CC6 produced a peak corresponding to (7), identified by comparison with an authentic standard.

FIG. 9 shows the characterization of SoUGT74CD1 and SoSDR. (A) Structure of 3-O-{β-D-xylopyranosyl-(1->3)-[β-D-galactopyranosyl-(1->2)]-3-D-glucopyranosiduronic acid}-28-O-{β-D-fucopyranosyl ester}-quillaic acid (QA-TriF, (8)), the product of SoUGT74CD1 when acting in combination with the S. officinalis enzymes required for production of 3-O-{β-D-xylopyranosyl-(1->3)-[β-D-galactopyranosyl-(1->2)]-3-D-glucopyranosiduronic acid}-quillaic acid (QA-Tri, (7)). Modification performed by SoUGT74CD1 has been highlighted and a table showing relevant calculated adducts and fragments of (8) included. (B) N. benthamiana leaves transiently co-expressing various genes were extracted and analysed using HPLC-MS, representative (n=6) extracted ion chromatograms (EIC) and MS/MS spectra are shown. EIC displayed are for m/z 1101.5118, the calculated mass of the [M-H]⁻ adduct of (8). The negative controls used were extracts from N. benthamiana leaves co-expressing only AstHMGR (tHMGR control) or co-expressing the S. officinalis genes required to produce (7) (tHMGR, SobAS1, SoCYP716A379, SoCYP72A984, SoCSL, SoUGT73DL1 and SoUGT73CC6) (QA-Tri). The additional activity of SoUGT74CD1 produced a peak corresponding to (8), identified by comparison with an authentic standard. Two additional smaller peaks (8′) and (8″), were also observed with the addition of SoUGT74CD1. Addition of SoSDR in combination with SoUGT74CD1 increased yields of peak (8) significantly but was not capable of producing (8) in the absence of SoUGT74CD1.

FIG. 10 shows the characterization of SoUGT79T1. (A) Structure of 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR, (9)), the product of SoUGT79T1 when acting in combination with the S. officinalis enzymes required for production of 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF, (8)). Modification performed by SoUGT79T1 has been highlighted and a table showing relevant calculated adducts and fragments of (9) included. (B) N. benthamiana leaves transiently co-expressing various genes were extracted and analysed using HPLC-MS, representative (n=6) extracted ion chromatograms (EIC) and MS/MS spectra are shown. EIC displayed are for m/z 1247.5679, the calculated mass of the [M-H]⁻ adduct of (9). The negative controls used were extracts from N. benthamiana leaves co-expressing only AstHMGR (tHMGR control) or co-expressing the S. officinalis genes required to produce (8) (tHMGR, SobAS1, SoCYP716A379, SoCYP72A984, SoCSL, SoUGT73DL1, SoUGT73CC6 and SoUGT74CD1) (QA-TriF). The additional activity of SoUGT79T1 produced a peak corresponding to (9) based on MSMS spectra, and later confirmed by comparison of downstream products to authentic standards.

FIG. 11 shows the characterization of SoUGT79L3. (A) Structure of 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX, (10 )), the product of SoUGT79L3 when acting in combination with the S. officinalis enzymes required for production of 3-O-{β-D-xylopyranosyl-(1->3)-[3-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR, (9)). Modification performed by SoUGT79L3 has been highlighted and a table showing relevant calculated adducts and fragments of (10) included. (B) N. benthamiana leaves transiently co-expressing various genes were extracted and analysed using HPLC-MS, representative (n=6) extracted ion chromatograms (EIC) and MS/MS spectra are shown. EIC displayed are for m/z 1379.6119, the calculated mass of the [M-H]⁻ adduct of (10). The negative controls used were extracts from N. benthamiana leaves co-expressing only AstHMGR (tHMGR control) or co-expressing the S. officinalis genes required to produce (9) (tHMGR, SobAS1, SoCYP716A379, SoCYP72A984, SoCSL, SOUGT73DL1, SoUGT73CC6, SoUGT74CD1 and SoUGT79T1) (QA-TriFR). The additional activity of SoUGT79L3 produced a peak corresponding to (10) based on MSMS spectra, and later confirmed by comparison of downstream products to authentic standards.

FIG. 12 shows the characterization of SoUGT73M2. (A) Structure of 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX, (11)), the product of SoUGT73M2 when acting in combination with the S. officinalis enzymes required for production of 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX, (10)). Modification performed by SoUGT73M2 has been highlighted and a table showing relevant calculated adducts and fragments of (11) included. (B) N. benthamiana leaves transiently co-expressing various genes were extracted and analysed using HPLC-MS, representative (n=6) extracted ion chromatograms (EIC) and MS/MS spectra are shown. EIC displayed are for m/z 1511.6542, the calculated mass of the [M-H]⁻ adduct of (11). The negative controls used were extracts from N. benthamiana leaves co-expressing only AstHMGR (tHMGR control) or co-expressing the S. officinalis genes required to produce (10) (tHMGR, SobAS1, SoCYP716A379, SoCYP72A984, SoCSL, SoUGT73DL1, SoUGT73CC6, SoUGT74CD1, SoUGT79T1 and SoUGT79L3) (QA-TriFRX). The additional activity of SoUGT73M2 produced a peak corresponding to (11) based on MSMS spectra, and later confirmed by comparison of downstream products to authentic standards.

FIG. 13 shows the characterization of SoGH1. (A) Structure of 3-O-{β-d-xylopyranosyl-(1→3)-[β-d-galactopyranosyl-(1→2)]-β-d-glucopyranosiduronic acid}-28-O-{β-d-xylopyranosyl-(1→3)-β-d-xylopyranosyl-(1→4)-α-I-rhamnopyranosyl-(1→2)-[3-d-quinovopyranosyl-(1→4)]-β-d-fucopyranosyl ester}-quillaic acid (QA-TriF(Q)RXX, (12)), the product of SoGH1 when acting in combination with the S. officinalis enzymes required for production of 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX, (11)). Modification performed by SoGH1 has been highlighted and a table showing relevant calculated adducts and fragments of (12) included. (B) N. benthamiana leaves transiently co-expressing various genes were extracted and analysed using HPLC-MS, representative (n=6) extracted ion chromatograms (EIC) and MS/MS spectra are shown. EIC displayed are for m/z 1657.7115, the calculated mass of the [M-H]⁻ adduct of (12). The negative controls used were extracts from N. benthamiana leaves co-expressing only AstHMGR (tHMGR control) or co-expressing the S. officinalis genes required to produce (11) (tHMGR, SobAS1, SoCYP716A379, SoCYP72A984, SoCSL, SOUGT73DL1, SoUGT73CC6, SoUGT74CD1, SoUGT79T1, SoUGT79L3 and SoUGT73M2) (QA-TriFRXX). The additional activity of SoGH1 produced a peak corresponding to (12) confirmed by comparison to authentic standards.

FIG. 14 shows the characterization of SoBHAD1. (A) Structure of saponarioside B (13), the product of SoBAHD1 when acting in combination with the S. officinalis enzymes 3-O-{β-d-xylopyranosyl-(1→3)-[β-d-galactopyranosyl-(1→2)]-β-d-glucopyranosiduronic acid}-28-O-{β-d-xylopyranosyl-(1→3)-3-d-xylopyranosyl-(1→4)-α-I-rhamnopyranosyl-(1→2)-[β-d-quinovopyranosyl-(1→4)]-β-d-fucopyranosyl ester}-quillaic acid (QA-TriF(Q)RXX, (12)). Modification performed by SoBAHD1 has been highlighted and a table showing relevant calculated adducts and fragments of (13) included. (B) N. benthamiana leaves transiently co-expressing various genes were extracted and analysed using HPLC-MS, representative (n=6) extracted ion chromatograms (EIC) and MS/MS spectra are shown. EIC displayed are for m/z 1699.7206, the calculated mass of the [M-H]⁻ adduct of (13). The negative controls used were extracts from N. benthamiana leaves co-expressing only AstHMGR (tHMGR control) or co-expressing the S. officinalis genes required to produce (12) (tHMGR, SobAS1, SoCYP716A379, SoCYP72A984, SoCSL, SoUGT73DL1, SoUGT73CC6, SoUGT74CD1, SoUGT79T1, SoUGT79L3, SoUGT73M2 and SoGH1) (QA-TriF(Q)RXX). The additional activity of SoBAHD1 produced a peak corresponding to (13) confirmed by comparison to authentic standards.

FIG. 15 shows the biosynthesis of saponarioside B. (A) Bar charts displaying the relative accumulation of (4-13) in N. benthamiana with the stepwise expression of each enzyme in the pathway. Relative accumulation is based on the integrated peak area of extracted ion chromatograms. Mean values are plotted and error bars representative of the mean, n=6. (B) Proposed biosynthetic pathway converting oxidosqualene to saponarioside B (a precursor to saponarioside A, and isomer of SO1861). Structures confirmed by comparison to authentic standards are indicated with a black circle. Order of steps drawn is only proposed, the actual order in planta may vary.

FIG. 16 shows the limited detection of β-amyrin (1) in both control DsRED and SobAS1 silenced hairy roots. The samples were extracted and analysed using GC/MS and the extraction ion chromatogram (EIC) at m/z 218 is shown. Commercial β-amyrin standard was used as comparison.

FIG. 17 shows the GC/MS analysis of soapwort hairy root samples in comparison with cycloartenol standard. The extracted ion chromatogram (EIC) at m/z 408 and the MS of the detected cycloartenol peaks are shown.

FIG. 18 shows the LC/MS analysis of soapwort hairy root samples in comparison with quillaic acid (4) standard. The extracted ion chromatogram (EIC) at m/z 485.3267 and the MS/MS fragmentation of detected peaks are shown.

FIG. 19 shows the LC/MS analysis of soapwort hairy root samples in comparison with saponarioside B (13) standard. The extracted ion chromatogram (EIC) at m/z 1699.7206 and the MS/MS fragmentation of detected peaks are shown.

### DETAILED DESCRIPTION

This invention relates to the production of triterpenoids, such as saponariosides and intermediates thereof, using biosynthetic enzymes encoded by newly characterised or identified genes from the Soapwort plant (Saponaria officinalis) and variants thereof. These enzymes may include β-amyrin synthase (bAS; SobAS; SEQ ID NO: 8), SoC28 oxidase (SoC28; SEQ ID NO: 2), SoC23 oxidase (SoC23; SEQ ID NO: 4), C28C16 oxidase (SoC28C16; SEQ ID NO: 6), QA 3-O glucuronosyl transferase (SoQA-GlcAT; SoCSL; SEQ ID NO: 10), QA-GlcA galactosyl transferase (SoC3Gal; SoC3Gal; SEQ ID NO: 12), QA-GlcA-Gal xylosyl transferase (SoQA-R XyIT; SoC3Xyl; SEQ ID NO: 14), QA-Tri fucosyl transferase (QATriFuT; SoC28F; SEQ ID NO: 16), QA-TriF rhamnosyl transferase (QA-TriFR; SoC28Rha; SEQ ID NO: 18), QA-TriFR xylosyl transferase (SoQA-TriFRXyIT; SoC28Xyl1; SEQ ID NO: 20), QA-TriFRX xylosyl transferase (SoQA-TriFRXXyIT; SoC28Xyl2; SEQ ID NO: 22), QA-TriFRXX quinovosyl transferase (SoGH1; SEQ ID NO: 34) and/or QA-TriF(Q)RXX acetyl transferase (SoBAHD1; SEQ ID NO: 36).

Each of the genes, polypeptide sequences and nucleotide sequences described herein is optionally obtained or derived from S officinalis.

The genes polypeptide sequences and nucleotide sequences described herein may be useful in the production of cyclic triterpenes, such as β-amyrin, oleanolic acid, echinocystic acid, quillaic acid (“QA”) and glycosylated forms of QA, such as saponariosides, QS-7, QS-21 and analogues and intermediates of these glycosylated forms of QA.

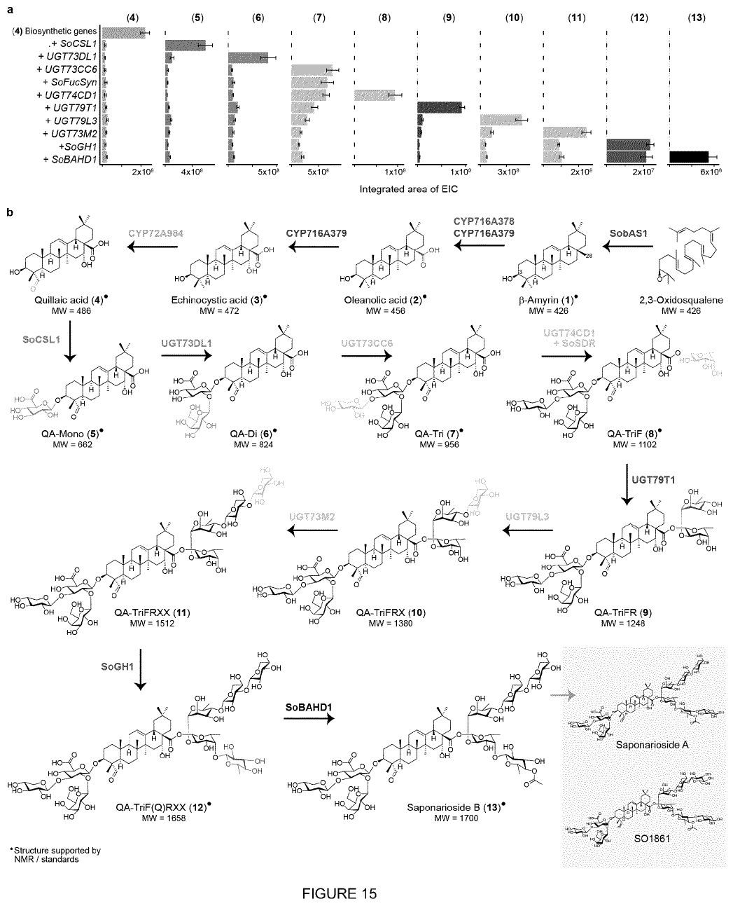

In some embodiments, one, two, three, four or more genes described herein may be useful in the production of quillaic acid (QA). QA is a derivative of the simple triterpene, β-amyrin, which is in turn synthesised by cyclisation of the universal linear precursor 2,3-oxidosqualene (OS) by oxidosqualene cyclases (OSCs). The β-amyrin scaffold is further oxidised with an alcohol, aldehyde and carboxylic acid at the C16, C-23 and C28 positions, respectively, to form QA. A proposed linear biosynthetic pathway is shown in FIG. 15, although the three oxidation reactions may equally occur in a different order, via the corresponding intermediates.

In preferred embodiments, QA may be produced from OS using genes encoding biosynthetic enzymes as set out below.

2,3-oxidosqualene (OS) may be converted into β-amyrin using Saponaria officinalis β-amyrin synthase (SobAS). SobAS may have the amino acid sequence of SEQ ID NO: 8 or may be a variant or fragment thereof.

Alternatively, 2,3-oxidosqualene (OS) may be converted into β-amyrin by an endogenous enzyme in a host cell.

The C28 position of β-amyrin may be oxidised to a carboxylic acid to produce oleanolic acid using a SoC28 oxidase (SoC28). SoC28 oxidase may have the amino acid sequence of SEQ ID NO: 2 or may be a variant or fragment thereof.

The C16 position of oleanolic acid may then be oxidised to an alcohol to produce echinocystic acid using a Saponaria officinalis C28C16 oxidase (SoC28C16). C28C16 oxidase may have the amino acid sequence of SEQ ID NO: 4 or may be a variant or fragment thereof.

Alternatively, the C28 position of β-amyrin may be oxidised to a carboxylic acid and the C16 position may be oxidised to an alcohol to produce echinocystic acid using a Saponaria officinalis C28C16 oxidase (SoC28C16). SoC28C16 oxidase may have the amino acid sequence of SEQ ID NO: 4 or may be a variant or fragment thereof.

The C-23 position of echinocystic acid may be oxidised to an aldehyde to produce QA using a SoC23oxidase (SoC23). SoC23 oxidase may have the amino acid sequence of SEQ ID NO: 6 or may be a variant or fragment thereof.

In some embodiments, genes described herein may be useful in the glycosylation of the C3 position of QA. Glycosylation may be initiated with a β-D-glucuronic acid (GlcA) residue attached at the 3-O position of QA. The GlcA residue is then linked to a D-Galactose (Gal) via a β-1->2 linkage and to a D-Xylose (Xyl) via a β-1,3 linkage.

In preferred embodiments, QA or C28 glycosylated forms of QA may be glycosylated at the 3-O position using genes encoding biosynthetic enzymes as set out below.

D-Glucuronic acid (“GlcA”) may be transferred to the 3-O position of quillaic acid to form 3-O-{β-D-glucopyranosiduronic acid}-quillaic acid (“QA-GlcA” or “QA-Mono”) using a Saponaria officinalis QA 3-O glucuronosyl transferase (“SoQA-GlcAT; SoCSL). The SoCSL may have the amino acid sequence of SEQ ID NO: 10 or may be a variant or fragment thereof.

D-Galactose (“Gal”) may be transferred via a β-1->2 linkage to QA mono (QA-GlcA) to form 3-O-{[β-D-galactopyranosyl-(1->2)]-β-D-glucopyranosiduronic acid}-quillaic acid (“QA-GlcA-Gal” or QA-Di) using a Saponaria officinalis QA-GlcA galactosyl transferase (“SoQA-GalT” or SoC3Gal). SoC3Gal may have the amino acid sequence of SEQ ID NO: 12 or may be a variant or fragment thereof.

D-Xylose (“Xyl”) may be transferred via a 1->3 linkage to QA-Di to form 3-O-{β-D-xylopyranosyl-(1->3)-[β-D-galactopyranosyl-(1->2)]-β-D-glucopyranosiduronic acid}-quillaic acid (“QA-GlcA-[Gal]-Xyl” or QA-Tri) using a Saponaria officinalis QA-GlcA-Gal Xylosyl transferase (“QA-XyIT” or SoC3Xyl). The QA-XyIT may have the amino acid sequence of SEQ ID NO: 14 or may be a variant or fragment thereof.

In some embodiments, genes described herein may be useful in the glycosylation of the C28 position of QA or C-3 glycosylated forms of QA.

D-Fucose (“Fuc”) may be transferred to the 28-O position of QA-Tri to form 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF) using a Saponaria officinalis QA-Tri fucosyl transferase (“SoQA-TriFuT” or “SoC28Fu”). SoC28Fu may have the amino acid sequence of SEQ ID NO: 16 or may be a variant or fragment thereof.

L-Rhamnose (“Rhap”) may be transferred via a β-1->2 linkage to QA-TriF to form 3-O-{β-D-xylopyranosyl-(1->3)-[3-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR) using a Saponaria officinalis QA-TriF rhamnosyl transferase (“SoQA-TriFRhaT” or “SoC28Rha”). SoC28Rha may have the amino acid sequence of SEQ ID NO: 18 or may be a variant or fragment thereof.

D-Xylose (“Xyl”) may be transferred via a 1->4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX) using a Saponaria officinalis QA-TriFR xylosyl transferase (“SoQA-TriFRXyIT” or SoC28Xyl1). SoC28Xyl1 may have the amino acid sequence of SEQ ID NO: 20 or may be a variant or fragment thereof.

D-Xylose (“Xyl”) may be transferred via a 1->3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX). using a Saponaria officinalis QA-TriFRX xylosyl transferase (“SoQA-TriFRXXyIT” or SoC28Xyl2). SoQA-TriFRXXyIT may have the amino acid sequence of SEQ ID NO: 22 or may be a variant or fragment thereof.

The quinovosyl group of QA-TriF(Q)RXX may be acetylated to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-2)-[β-D-4-O-acetylquinovopyranosyl-(1->4)]-β-D-fucopyranosyl ester}-quillaic acid (SpB) using a Saponaria officinalis QA-TriF(Q)RXX acetyl transferase (“SoBAHD1”) polypeptide. SoBAHD1 may have the amino acid sequence of SEQ ID NO: 36 or may be a variant or fragment thereof.

In preferred embodiments, the methods described herein will include the use of one or more of these newly characterised triterpenoid biosynthetic nucleic acids (e.g. one, two, three or more such nucleic acids) optionally in conjunction with the manipulation of other genes affecting QA or glycosylated QA biosynthesis known in the art.

These newly characterised triterpenoid biosynthetic amino acid and nucleotide sequences from Saponaria officinalis (SEQ. ID: Nos 1-22, and 33-36) form aspects of the invention in their own right, as do variants of these sequences and methods of using them. Any one of these sequences or variants may be used to alter the QA or glycosylated QA content of a plant, as disclosed herein. For instance, a variant nucleic acid may include a sequence encoding a variant polypeptide sharing the relevant biological activity of the native polypeptide, as discussed above. Examples include variants of any of SEQ ID Nos 1 to 22 and 33-36. For brevity, in the context of the present invention, and in particular the methods and uses described herein, the polypeptide or nucleotide sequences of SEQ ID NOs: 1 to 22 and 33-36 and variants thereof described herein may be referred to herein as “triterpenoid biosynthetic sequences” e.g. triterpenoid biosynthetic genes and triterpenoid biosynthetic polypeptides.

Provided herein is a Saponaria officinalis β-amyrin synthase (SobAS) polypeptide having the amino acid sequence of SEQ ID NO: 8 or a variant thereof, for example an amino acid sequence with at least 80% sequence identity to SEQ ID NO: 8. Also provided herein is a nucleic acid encoding said SobAS polypeptide having the nucleotide sequence of SEQ ID NO: 7 or a variant thereof, for example a nucleotide sequence with at least 80% sequence identity to SEQ ID NO: 7; and a vector comprising said nucleic acid. The SobAS polypeptide may be capable of cyclisation of the universal linear precursor 2,3-oxidosqualene (OS) to a triterpene.

Also provided herein is a SoC28 oxidase polypeptide having the amino acid sequence of SEQ ID NO: 2 or a variant thereof, for example an amino acid sequence with at least 80% sequence identity to SEQ ID NO: 2. Also provided herein is a nucleic acid encoding said SoC28 oxidase polypeptide having the nucleotide sequence of SEQ ID NO: 1 or a variant thereof, for example a nucleotide sequence with at least 80% sequence identity to SEQ ID NO: 1; and a vector comprising said nucleic acid. The SoC28 oxidase polypeptide may be capable of oxidising β-amyrin at the C28 position to a carboxylic acid forming oleanolic acid.

Also provided herein is a SoC28C16 oxidase polypeptide having the amino acid sequence of SEQ ID NO: 4 or a variant thereof, for example an amino acid sequence with at least 50% sequence identity to SEQ ID NO: 4. Also provided herein is a nucleic acid encoding said SoC28C16 oxidase polypeptide having the nucleotide sequence of SEQ ID NO: 3 or a variant thereof, for example a nucleotide sequence with at least 50% sequence identity to SEQ ID NO: 3; and a vector comprising said nucleic acid. The SoC28C16 oxidase polypeptide may be capable of oxidising β-amyrin, at the C16 position to an alcohol and at the C28 position to a carboxylic acid to form echinocystic acid.

Also provided herein is a SoC23 oxidase polypeptide having the amino acid sequence of SEQ ID NO: 6 or a variant thereof, for example an amino acid sequence with at least 50% sequence identity to SEQ ID NO: 6. Also provided herein is a nucleic acid encoding said SoC23 oxidase polypeptide having the nucleotide sequence of SEQ ID NO: 5 or a variant thereof, for example a nucleotide sequence with at least 50% sequence identity to SEQ ID NO: 5; and a vector comprising said nucleic acid. The SoC23 oxidase may be capable of oxidising echinocystic acid at the C-23 position to an aldehyde forming QA

Also provided herein is a Saponaria officinalis QA 3-O glucuronosyl transferase (“SoCSL”) polypeptide having the amino acid sequence of SEQ ID NO: 10 or a variant thereof, for example an amino acid sequence with at least 60% sequence identity to SEQ ID NO: 10. Also provided herein is a nucleic acid encoding said QA 3-O glucuronosyl transferase (“SoCSL”) polypeptide having the nucleotide sequence of SEQ ID NO: 9 or a variant thereof, for example a nucleotide sequence with at least 60% sequence identity to SEQ ID NO: 9; and a vector comprising said nucleic acid. The SoCSL may be capable of attaching D-glucuronic acid (“GlcA”) to the 3-O position of quillaic acid to form 3-O-{β-D-glucopyranosiduronic acid}-quillaic acid (“QA-GlcA” or “QA-Mono”).

Also provided herein is a Saponaria officinalis QA-GlcA galactosyl transferase (“SoC3Gal”) polypeptide having the amino acid sequence of SEQ ID NO: 12 or a variant thereof, for example an amino acid sequence with at least 80% sequence identity to SEQ ID NO: 12. Also provided herein is a nucleic acid encoding said Saponaria officinalis QA-GlcA galactosyl transferase (“SoC3Gal”) polypeptide having the nucleotide sequence of SEQ ID NO: 11 or a variant thereof, for example a nucleotide sequence with at least 80% sequence identity to SEQ ID NO: 11; and a vector comprising said nucleic acid. The SoC3Gal may be capable of attaching D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-Di” or “QA-GlcA-Gal”).

Also provided herein is a Saponaria officinalis QA-GlcA-Gal xylosyl transferase (“SoC3Xyl”) polypeptide having the amino acid sequence of SEQ ID NO: 14 or a variant thereof, for example an amino acid sequence with at least 80% sequence identity to SEQ ID NO: 14. Also provided herein is a nucleic acid encoding said Saponaria officinalis QA-GlcA-Gal xylosyl transferase (“SoC3Xyl”) polypeptide having the nucleotide sequence of SEQ ID NO: 13 or a variant thereof, for example a nucleotide sequence with at least 80% sequence identity to SEQ ID NO: 13; and a vector comprising said nucleic acid. The SoC3Xyl may be capable of attaching D-Xylose (“Xyl”) via a 1,3 linkage to QA-GlcA-Gal to form 1, 3-O-{β-D-xylopyranosyl-(1->3)-[3-D-galactopyranosyl-(1->2)]-β-D-glucopyranosiduronic acid}-quillaic acid (“QA-GlcAGalXyl” or “QA-Tri”),

Also provided herein is a Saponaria officinalis QA-Tri fucosyl transferase (“SoC28Fu”) polypeptide having the amino acid sequence of SEQ ID NO: 16 or a variant thereof, for example an amino acid sequence with at least 60% sequence identity to SEQ ID NO: 16. Also provided herein is a nucleic acid encoding said Saponaria officinalis QA-Tri fucosyl transferase (“SoC28Fu”) polypeptide having the nucleotide sequence of SEQ ID NO: 15 or a variant thereof, for example a nucleotide sequence with at least 60% sequence identity to SEQ ID NO: 15; and a vector comprising said nucleic acid. The SoC28Fu may be capable of attaching fucose(“Fuc”) to the 28-O position of QA-Tri to form 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF).

Also provided herein is a Saponaria officinalis QA-TriF rhamnosyl transferase (“SoC28Rha”) polypeptide having the amino acid sequence of SEQ ID NO: 18 or a variant thereof, for example an amino acid sequence with at least 50% sequence identity to SEQ ID NO: 18. Also provided herein is a nucleic acid encoding said Saponaria officinalis QA-TriF rhamnosyl transferase (“SoC28Rha”) polypeptide having the nucleotide sequence of SEQ ID NO: 17 or a variant thereof, for example a nucleotide sequence with at least 50% sequence identity to SEQ ID NO: 17; and a vector comprising said nucleic acid. The SoC28Rha may be capable of attaching rhamnose (“Rha”) via a 1, 2 linkage to QA-TriF to form 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR).

Also provided herein is a Saponaria officinalis QA-TriFR xylosyl transferase (“SoC28Xyl1”) polypeptide having the amino acid sequence of SEQ ID NO: 20 or a variant thereof, for example an amino acid sequence with at least 50% sequence identity to SEQ ID NO: 20. Also provided herein is a nucleic acid encoding said Saponaria officinalis QA-TriFR xylosyl transferase (“SoC28Xyl1”) polypeptide having the nucleotide sequence of SEQ ID NO: 19 or a variant thereof, for example a nucleotide sequence with at least 50% sequence identity to SEQ ID NO: 19; and a vector comprising said nucleic acid. The SoC28Xyl1 may be capable of attaching D-Xylose (“Xyl”) via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX).

Also provided herein is a Saponaria officinalis QA-TriFRX xylosyl transferase (“SoC28Xyl2”) polypeptide having the amino acid sequence of SEQ ID NO: 22 or a variant thereof, for example an amino acid sequence with at least 50% sequence identity to SEQ ID NO: 22. Also provided herein is a nucleic acid encoding said Saponaria officinalis QA-TriFRX xylosyl transferase (“SoC28Xyl2”) polypeptide having the nucleotide sequence of SEQ ID NO: 21 or a variant thereof, for example a nucleotide sequence with at least 50% sequence identity to SEQ ID NO: 21; and a vector comprising said nucleic acid. The SoC28Xyl2may be capable of attaching D-Xylose (“Xyl”) via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX).

Also provided herein is a Saponaria officinalis QA-TriFRXX quinovosyl transferase (“SoGH1”) polypeptide having the amino acid sequence of SEQ ID NO: 34 or a variant thereof, for example an amino acid sequence with at least 50% sequence identity to SEQ ID NO: 34. Also provided herein is a nucleic acid encoding said

Saponaria officinalis QA-TriFRXX quinovosyl transferase (“SoGH1”) polypeptide having the nucleotide sequence of SEQ ID NO: 33 or a variant thereof, for example a nucleotide sequence with at least 50% sequence identity to SEQ ID NO: 33; and a vector comprising said nucleic acid. The SoGH1 may be capable of attaching D-Quinovose (“Q”) via a 1,4 linkage to QA-TriFRXX to form 3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[β-D-quinovopyranosyl-(1→4)]-β-D-fucopyranosyl ester}-quillaic acid (QA-TriF(Q)RXX).

Also provided herein is a Saponaria officinalis QA-TriF(Q)RXX acetyl transferase (“SoBAHD1”) polypeptide having the amino acid sequence of SEQ ID NO: 36 or a variant thereof, for example an amino acid sequence with at least 50% sequence identity to SEQ ID NO: 36. Also provided herein is a nucleic acid encoding said Saponaria officinalis QA-TriF(Q)RXX acetyl transferase (“SoBAHD1”) polypeptide having the nucleotide sequence of SEQ ID NO: 35 or a variant thereof, for example a nucleotide sequence with at least 50% sequence identity to SEQ ID NO: 35; and a vector comprising said nucleic acid. The SoBAHD1 may be capable of acetylating QA-TriF(Q)RXX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-[β-D-4-O-acetylquinovopyranosyl-(1→4)]-β-D-fucopyranosyl ester}-quillaic acid (SpB).

An amino acid sequence described herein that is a variant of a reference sequence, such as a peptide, polypeptide or protein sequence described herein, for example any one of SEQ ID NOs: 2, 4, 6, 8, 10, 12, 14, 16, 18, 20, 22, 34 or 36, may have 1 or more amino acid residues altered relative to the reference sequence. For example, 50 or fewer amino acid residues may be altered relative to the reference sequence, preferably 45 or fewer, 40 or fewer, 30 or fewer, 20 or fewer, 15 or fewer, 10 or fewer, 5 or fewer or 3 or fewer, 2 or 1. For example, a variant described herein may comprise the sequence of a reference sequence with 50 or fewer, 45 or fewer, 40 or fewer, 30 or fewer, 20 or fewer, 15 or fewer, 10 or fewer, 5 or fewer, 3 or fewer, 2 or 1 amino acid residues mutated.

An amino acid residue in the reference sequence may be altered or mutated by insertion, deletion or substitution, preferably substitution for a different amino acid residue. Such alterations may be caused by one or more of addition, insertion, deletion or substitution of one or more nucleotides in the encoding nucleic acid.

A nucleotide sequence described herein that is a variant of a reference sequence, such as a nucleotide sequence described herein, for example any one of SEQ ID NOs: 1, 3, 5, 7, 9, 11, 13, 15, 17, 19,21, 33 or 35, may have 1 or more nucleotides altered relative to the reference sequence. For example, 50 or fewer nucleotides may be altered relative to the reference sequence, preferably 45 or fewer, 40 or fewer, 30 or fewer, 20 or fewer, 15 or fewer, 10 or fewer, 5 or fewer or 3 or fewer, 2 or 1. For example, a variant described herein may comprise the sequence of a reference sequence with 50 or fewer, 45 or fewer, 40 or fewer, 30 or fewer, 20 or fewer, 15 or fewer, 10 or fewer, 5 or fewer, 3 or fewer, 2 or 1 nucleotides mutated.

A peptide, polypeptide or protein as described herein or a nucleotide sequence as described herein that is a variant of a reference sequence, such as an amino acid or nucleotide sequence described above, may share at least 50% sequence identity with the reference sequence, at least 55%, at least 60%, at least 65%, at least 70%, at least about 80%, at least 90%, at least 95%, at least 98% or at least 99% sequence identity. For example, a variant of a protein described herein may comprise an amino acid sequence that has at least 50% sequence identity with the reference amino acid sequence, at least 55%, at least 60%, at least 65%, at least 70%, at least about 80%, at least 90%, at least 95%, at least 98% or at least 99% sequence identity with the reference amino acid sequence, for example one or more of SEQ ID NOs: 2, 4, 6, 8, 10, 12, 14, 16, 18, 20,22, 34 and 36.

A variant of a nucleic acid described herein may comprise a nucleotide sequence that has at least 50% sequence identity with the reference amino acid sequence, at least 55%, at least 60%, at least 65%, at least 70%, at least about 80%, at least 90%, at least 95%, at least 98% or at least 99% sequence identity with the reference nucleotide sequence, for example one or more of SEQ ID NOs: 1, 3, 5, 7, 9, 11, 13, 15, 17, 19, 21, 33 and 35.

Variants of different variant triterpenoid biosynthetic sequences may share different levels of sequence identity to their respective reference sequences. Combinations of variant triterpenoid biosynthetic sequences with all levels of sequence identity disclosed above are encompassed by the invention.

Sequence identity is commonly defined with reference to the algorithm GAP (Wisconsin GCG package,

Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm may be used (Nucl. Acids Res. (1997) 25 3389-3402). Sequence identity and similarity may also be determined using Genomequest™ software (Gene-IT, Worcester MA USA).

Sequence comparisons are preferably made over the full-length of the relevant sequence described herein.

A variant polypeptide may share the relevant biological activity of the reference polypeptide. A variant nucleic acid may encode the relevant variant polypeptide. In this context, the “biological activity” of a polypeptide described herein is the ability to catalyse the respective reaction shown in FIG. 15 and described above. The relevant biological activities may be assayed based on the reactions shown in FIG. 15 in vitro. Alternatively, they can be assayed by activity in vivo as described in the Examples i.e. by introduction of a plurality of heterologous constructs to generate the respective product into a host, which can be assayed by LC-MS or the like.

Preferred variants may be:
(i) Naturally occurring nucleic acids such as alleles (which will include polymorphisms or mutations at one or more bases) or pseudoalleles (which may occur at closely linked loci to the biosynthetic genes described herein). Also included are paralogues, isogenes, or other homologous genes belonging to the same families as the biosynthetic genes described herein, for example sharing clades or sub-clades. Also included are orthologues or homologues from other plant species (i.e., plants other than S. officinalis) Homology may be at the nucleotide sequence and/or amino acid sequence level, as discussed below.
(ii) Artificial nucleic acids, which can be prepared by the skilled person in the light of the present disclosure. Such derivatives may be prepared, for instance, by site directed or random mutagenesis, or by direct synthesis. Preferably the variant nucleic acid is generated either directly or indirectly (e.g. via one or more amplification or replication steps) from an original nucleic acid having all or part of the sequence of a biosynthetic gene described herein.

Variants may also include nucleic acids corresponding to those above, but which have been extended at the 3′ or 5′ terminus.

A method of producing a variant triterpenoid biosynthetic nucleic acid may comprise the step of modifying any of the genes described herein, for example one or more of SEQ ID NOs: 1, 3, 5, 7, 9, 11, 13, 15, 17, 19 21, 33 and 35.

Changes may be desirable for a number of reasons. For instance, they may introduce or remove restriction endonuclease sites or alter codon usage. This may be particularly desirable where the genes are to be expressed in alternative hosts e.g. microbial hosts such as yeast. Methods of codon optimizing genes for this purpose are known in the art (see e.g. Elena, Claudia, et al. “Expression of codon optimized genes in microbial systems: current industrial applications and perspectives.” Frontiers in microbiology 5 (2014)). Sequences described herein including codon modifications to maximise yeast expression represent embodiments of the invention.

Alternatively, changes to a sequence may produce a derivative by way of one or more (e.g. several) of addition, insertion, deletion or substitution of one or more nucleotides in the nucleic acid, leading to the addition, insertion, deletion or substitution of one or more (e.g. several) amino acids in the encoded polypeptide.

Such changes may modify sites which are required for post translation modification such as cleavage sites in the encoded polypeptide; motifs in the encoded polypeptide for phosphorylation etc. Leader or other targeting sequences (e.g. membrane or golgi locating sequences) may be added to the expressed protein to determine its location following expression if it is desired to isolate it from a microbial system.

Other desirable mutations may be random or site-directed mutagenesis in order to alter the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation. Also included are variants having non-conservative substitutions. As is well known to those skilled in the art, substitutions to regions of a peptide which are not critical in determining its conformation may not greatly affect its activity because they do not greatly alter the peptide's three-dimensional structure. In regions which are critical in determining the peptides conformation or activity such changes may confer advantageous properties on the polypeptide. Indeed, changes such as those described above may confer slightly advantageous properties on the peptide e.g. altered stability or specificity.

In some embodiments, a variant nucleotide sequence encoding a So polypeptide may be obtainable by means of a method which includes:
(a) providing a preparation of nucleic acid, e.g. from plant cells. Test nucleic acid may be provided from a cell as genomic DNA, cDNA or RNA, or a mixture of any of these, preferably as a library in a suitable vector. If genomic DNA is used the probe may be used to identify untranscribed regions of the gene (e.g. promoters etc.), such as are described hereinafter,
(b) providing a nucleic acid molecule which is a probe or primer as discussed above,
(c) contacting nucleic acid in said preparation with said nucleic acid molecule under conditions for hybridisation of said nucleic acid molecule to any said gene or homologue in said preparation, and,
(d) identifying said gene or homologue if present by its hybridisation with said nucleic acid molecule. Binding of a probe to target nucleic acid (e.g. DNA) may be measured using any of a variety of techniques at the disposal of those skilled in the art. For instance, probes may be radioactively, fluorescently, or enzymatically labelled. Other methods not employing labelling of probe include amplification using PCR (see below), RNase cleavage and allele specific oligonucleotide probing. The identification of successful hybridisation is followed by isolation of the nucleic acid which has hybridised, which may involve one or more steps of PCR or amplification of a vector in a suitable host.

Preliminary experiments may be performed by hybridising under low stringency conditions. For probing, preferred conditions are those which are stringent enough for there to be a simple pattern with a small number of hybridisations identified as positive which can be investigated further.

For example, hybridizations may be performed, according to the method of Sambrook et al. (below) using a hybridization solution comprising: 5× SSC (wherein ‘SSC’=0.15 M sodium chloride; 0.15 M sodium citrate; pH 7), 5× Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2× SSC and 1% SDS; (2) 15 minutes at room temperature in 2× SSC and 0.1% SDS; (3) 30 minutes-1 hour at 37° C. in 1× SSC and 1% SDS; (4) 2 hours at 42-65° C. in 1× SSC and 1% SDS, changing the solution every 30 minutes.

One common formula for calculating the stringency conditions required to achieve hybridization between nucleic acid molecules of a specified sequence homology is (Sambrook et al., 1989): Tₘ=81.5° C.+16.6Log [Na+]+0.41 (% G+C)−0.63 (% formamide)−600/#bp in duplex

As an illustration of the above formula, using [Na+]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

It is well known in the art to increase stringency of hybridisation gradually until only a few positive clones remain. Other suitable conditions include, e.g. for detection of sequences that are about 80-90% identical, hybridization overnight at 42° C. in 0.25M Na2HPO4, pH 7.2, 6.5% SDS, 10% dextran sulfate and a final wash at 55° C. in 0.1× SSC, 0.1% SDS. For detection of sequences that are greater than about 90% identical, suitable conditions include hybridization overnight at 65° C. in 0.25M Na2HPO4, pH 7.2, 6.5% SDS, 10% dextran sulfate and a final wash at 60° C. in 0.1× SSC, 0.1% SDS.

In a further embodiment, hybridization of a triterpenoid biosynthetic nucleic acid molecule to a variant may be determined or identified indirectly, e.g. using a nucleic acid amplification reaction, particularly the polymerase chain reaction (PCR). PCR requires the use of two primers to specifically amplify target nucleic acid, so preferably two nucleic acid molecules with sequences characteristic of a triterpenoid biosynthetic gene are employed. Using RACE PCR, only one such primer may be needed (see “PCR protocols; A Guide to Methods and Applications”, Eds. Innis et al, Academic Press, New York, (1990)).

Thus, a method involving use of PCR in obtaining a variant triterpenoid biosynthetic nucleic acid as described herein may include:
(a) providing a preparation of plant nucleic acid, e.g. from a seed or other appropriate tissue or organ,
(b) providing a pair of nucleic acid molecule primers useful in (i.e. suitable for) PCR, at least one of said primers being a primer directed to a triterpenoid biosynthetic sequence as discussed above,
(c) contacting nucleic acid in said preparation with said primers under conditions for performance of PCR,
(d) performing PCR and determining the presence or absence of an amplified PCR product. The presence of an amplified PCR product may indicate identification of a variant.

In all cases above, if need be, clones or fragments identified in the search can be extended. For instance, if it is suspected that they are incomplete, the original DNA source (e.g. a clone library, mRNA preparation etc.) can be revisited to isolate missing portions e.g. using sequences, probes or primers based on that portion which has already been obtained to identify other clones containing overlapping sequence.

The methods described herein may utilise fragments of the triterpenoid biosynthetic genes described herein, for example one or more of SEQ ID NOs: 1, 3, 5, 7, 9, 11, 13, 15, 17, 19, 21, 33 and 35; or fragments of variants of these genes. Also provided is the production and use of fragments of the full-length polypeptides disclosed herein, especially active portions thereof. An “active portion” of a polypeptide means a peptide which is less than said full length polypeptide, but which retains its essential biological activity e.g. in relation to production of QA or the glycosylation of QA.

A fragment of a full-length reference triterpenoid biosynthetic polypeptide sequence, such as SEQ ID NOs: 2, 4, 6, 8, 10, 12, 14, 16, 18, 20, 22, 34 or 36, is a contiguous sequence of amino acids from the full-length protein sequence that consists of at least one fewer amino acid than the full-length protein sequence. For example, a fragment may lack a sequence of 10 or more, 20 or more, 50 or more of 100 or more amino acids relative to the full-length sequence. Preferably a fragment shares the relevant biological activity of the full-length reference polypeptide.

In some embodiments, fragments of the polypeptides may include one or more epitopes useful for raising antibodies to a portion of any of the amino acid sequences disclosed herein. Preferred epitopes are those to which antibodies are able to bind specifically, which may be taken to be binding a polypeptide or fragment thereof with an affinity which is at least about 1000× that of other polypeptides.

Purified protein (polypeptide, enzyme), or a fragment, mutant, derivative or variant thereof, e.g. produced recombinantly by expression from encoding triterpenoid biosynthetic nucleic acid therefor, forms an aspect of the invention.

Such purified polypeptides may be used to raise antibodies employing techniques which are standard in the art. Antibodies and polypeptides comprising antigen-binding fragments of antibodies may be used in identifying homologues from other species as discussed further below.

Methods of producing antibodies include immunising a mammal (e.g. human, mouse, rat, rabbit, horse, goat, sheep or monkey) with the protein or a fragment thereof. Antibodies may be obtained from immunised animals using any of a variety of techniques known in the art, and might be screened, preferably using binding of antibody to antigen of interest. For instance, Western blotting techniques or immunoprecipitation may be used (Armitage et al, 1992, Nature 357: 80-82). Antibodies may be polyclonal or monoclonal.

As an alternative or supplement to immunising a mammal, antibodies with appropriate binding specificity may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies raised to a polypeptide or peptide can be used in the identification and/or isolation of homologous polypeptides, and then the encoding genes.

Antibodies may be modified in a number of ways. Indeed the term “antibody” should be construed as covering any specific binding substance having a binding domain with the required specificity. Thus, this term covers antibody fragments, derivatives, functional equivalents and homologues of antibodies, including any polypeptide comprising an immunoglobulin binding domain, whether natural or synthetic.

Mevalonic acid (MVA) is an important intermediate in triterpenoid synthesis. Therefore, it may be desirable to express rate limiting MVA pathway genes into the host, to maximise yields of a triterpenoid, such as QA. HMG-CoA reductase (HMGR) is believed to be a rate-limiting enzyme in the MVA pathway.

The use of a recombinant feedback-insensitive truncated form of HMGR (tHMGR) has been demonstrated to increase triterpene (β-amyrin) content upon transient expression in N. benthamiana [Reed, J., et al. Metab Eng, 2017. 42: p. 185-193].

In some embodiments, a heterologous HMGR (e.g. a feedback insensitive HMGR) may be used along with the triterpenoid biosynthetic genes described herein.

Examples of HMGR encoding or polypeptide sequences include SEQ ID Nos 23-26, or variants or fragments of these. Variants may be homologues, alleles, or artificial derivatives etc. as discussed in relation to biosynthetic genes or polypeptides as described above. For example an HMGR native to the host being utilised may be preferred—for example a yeast HMGR in a yeast host, and so on. HMGR genes are known in the art and may be selected, as appropriate in the light of the present disclosure.

It has also been reported that squalene synthase (SQS) is a potential rate-limiting step [Reed et al supra].

In some embodiments, a heterologous SQS may be used along with the biosynthetic genes described herein and optionally HMGR described herein.

Examples of SQS encoding or polypeptide sequences include SEQ ID Nos 27 and 28, or variants or fragments of these. Variants may be homologues, alleles, or artificial derivatives etc. as discussed in relation to biosynthetic genes or polypeptides as described above. For example an SQS native to the host being utilised may be preferred—for example a yeast SQS in a yeast host, and so on. SQS genes are known in the art and may be selected, as appropriate in the light of the present disclosure.

When using certain hosts (for example yeasts) it may be desirable to introduce additional genes to improve the flux of biosynthetic production. Examples may include one or more plant cytochrome P450 reductases (CPRs) to serve as the redox partner to the introduced P450s. In some embodiments, a heterologous cytochrome P450 reductase such as AtATR2 (Arabidopsis thaliana cytochrome P450 reductase 2) may be used along with the biosynthetic polypeptides and genes described herein. Examples of AtATR2 encoding or polypeptide sequences include SEQ ID Nos 29 and 30, or variants or fragments of these. Variants may be homologues, alleles, or artificial derivatives etc. as discussed in relation to biosynthetic polypeptides and genes as described above.

In some embodiments, a heterologous nucleic acid described herein may further encode one or more of the following polypeptides: (i) an HMG-COA reductase (HMGR) and/or (ii) a squalene synthase (SQS). HMGR or SQS may be optionally selected from the respective polypeptides in SEQ ID NOs 24, 26 and 28 or variants or fragments of any of said polypeptides or are encoded by the respective polynucleotides of SEQ ID NOS 23, 25 and 27, or variants or fragments of any of said polynucleotides.

Nucleic acid may include cDNA, RNA, genomic DNA and modified nucleic acids or nucleic acid analogues (e.g. peptide nucleic acid). Where a DNA sequence is specified, e.g. with reference to a figure, unless context requires otherwise the RNA equivalent, with U substituted for T where it occurs, is encompassed. Nucleic acids may include more than one nucleic acid molecule. Nucleic acid molecules according to the present invention may be provided isolated and/or purified from their natural environment, in substantially pure or homogeneous form, or free or substantially free of other nucleic acids of the species of origin, and double or single stranded. Where used herein, the term “isolated” encompasses all of these possibilities. The nucleic acid molecules may be wholly or partially synthetic. In particular they may be recombinant in that nucleic acid sequences which are not found together in nature (do not run contiguously) have been ligated or otherwise combined artificially. Nucleic acids may comprise, consist, or consist essentially of, any of the sequences discussed hereinafter.

The complement of a nucleic acid described herein means the complementary sequence of the or a nucleotide sequence comprised by the nucleic acid. Optionally, complementary sequences are full length compared to the reference nucleotide sequence.

The term “heterologous” is used broadly herein to indicate that the gene/sequence of nucleotides in question (e.g. encoding biosynthesis modifying polypeptides) have been introduced into said cells of the host or an ancestor thereof, using genetic engineering, i.e. by human intervention. Nucleic acid heterologous to a host cell will be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

“Transformed” in this context means that the nucleotide sequences of the heterologous nucleic acid alter one or more of the cell's characteristics and hence phenotype e.g. with respect to the ability to biosynthesise a triterpenoid, such as QA or glycosylated QA e.g. QATri, QATriFRXX, QATriF(Q)RXX or SpB. Such transformation may be transient or stable.

“Unable to carry out biosynthesis” means that the host, prior to the conversion, does not, or is not believed to, naturally produce detectable or recoverable levels of product under normal metabolic circumstances of that host. Following the application of the invention it is able to produce detectable or recoverable levels of product

The nucleotide sequence information provided herein may be used to design probes and primers for probing or amplification. An oligonucleotide for use in probing or PCR may be about 30 or fewer nucleotides in length (e.g. 18, 21 or 24). Generally specific primers are upwards of 14 nucleotides in length. For optimum specificity and cost effectiveness, primers of 16-24 nucleotides in length may be preferred. Those skilled in the art are well versed in the design of primers for use in processes such as PCR. If required, probing can be done with entire restriction fragments of the gene disclosed herein which may be 100's or even 1000's of nucleotides in length. Small variations may be introduced into the sequence to produce ‘consensus’ or ‘degenerate’ primers if required.

Probing may employ the standard Southern blotting technique. For instance, DNA may be extracted from cells and digested with different restriction enzymes. Restriction fragments may then be separated by electrophoresis on an agarose gel before denaturation and transfer to a nitrocellulose filter. Labelled probe may be hybridised to the single stranded DNA fragments on the filter and binding determined. DNA for probing may be prepared from RNA preparations from cells. Probing may optionally be done by means of so-called ‘nucleic acid chips’ (see Marshall & Hodgson (1998) Nature Biotechnology 16: 27-31, for a review).

A method described herein may employ the co-infiltration of a plurality of Agrobacterium tumefaciens strains each carrying one or more of the triterpenoid biosynthetic genes discussed above for concerted expression thereof in a biosynthetic pathway discussed above.

In some embodiments, at least 2 or 3 different Agrobacterium tumefaciens strains are co-infiltrated e.g. each carrying a triterpenoid biosynthetic nucleic acid.

The genes may be present from transient expression vectors.

Vectors (typically binary vectors) for use as described herein may typically comprise an expression cassette comprising:
(i) a promoter, operably linked to
(ii) an enhancer sequence derived from the RNA-2 genome segment of a bipartite RNA virus, in which a target initiation site in the RNA-2 genome segment has been mutated;
(iii) a nucleic acid sequence encoding one or more biosynthetic genes as described above;
(iv) a terminator sequence; and optionally
(v) a 3′ UTR located upstream of said terminator sequence.

Further examples of vectors and expression systems suitable for use as described herein are described below.

A triterpenoid biosynthetic gene described above may be contained in or in the form of a recombinant and preferably replicable vector. A vector may include, inter alia, any plasmid, cosmid, phage or Agrobacterium binary vector in double or single stranded linear or circular form which may or may not be self-transmissible or mobilizable, and which can transform a prokaryotic or eukaryotic host either by integration into the cellular genome or exist extrachromosomally (e.g. autonomous replicating plasmid with an origin of replication).

Suitable expression vectors may include binary vectors for transient expression mediated by Agrobacterium tumefaciens (see for example Bevan et al Nucl Acid Res 984 November 26; 12(22): 8711-872).

As is well known to those skilled in the art, a “binary vector” system includes (a) border sequences which permit the transfer of a desired nucleotide sequence into a plant cell genome; (b) desired nucleotide sequence itself, which will generally comprise an expression cassette of (i) a plant active promoter, operably linked to (ii) the target sequence and/or enhancer as appropriate. The desired nucleotide sequence is situated between the border sequences and is capable of being inserted into a plant genome under appropriate conditions. The binary vector system will generally require other sequence (derived from A. tumefaciens) to effect the integration. Generally this may be achieved by use of so called “agro-infiltration” which uses Agrobacterium-mediated transient transformation. Briefly, this technique is based on the property of Agrobacterium tumefaciens to transfer a portion of its DNA (“T-DNA”) into a host cell where it may become integrated into nuclear DNA. The T-DNA is defined by left and right border sequences which are around 21-23 nucleotides in length. The infiltration may be achieved e.g. by syringe (in leaves) or vacuum (whole plants). In the present invention the border sequences will generally be included around the desired nucleotide sequence (the T-DNA) with the one or more vectors being introduced into the plant material by agro-infiltration.

Other suitable expression systems may utilise the called “Hyper-Translatable’ Cowpea Mosaic Virus (‘CPMV-HT’) system. Suitable vectors based on pEAQ-HT expression plasmids for use in the CPMV-HT system are well known in the art (see for example WO2009/087391; Sainsbury et al (2009) Plant Biotechnol J 7(7): 682-693)

Generally speaking, those skilled in the art are well able to construct vectors and design protocols for recombinant gene expression (e.g. for expressing a heterologous nucleic acid within a host or one or more cells of a host). Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

Specifically included are shuttle vectors by which is meant a DNA vehicle capable, naturally or by design, of replication in two different host organisms, which may be selected from actinomycetes and related species, bacteria and eucaryotic (e.g. higher plant, mosses, yeast or fungal cells).

A vector including nucleic acid described herein need not include a promoter or other regulatory sequence, particularly if the vector is to be used to introduce the nucleic acid into cells for recombination into the genome.

Preferably the nucleic acid in the vector is under the control of, and operably linked to, an appropriate promoter or other regulatory elements for transcription in a host cell such as a microbial, e.g. yeast and bacterial, or plant cell. The vector may be a bi-functional expression vector which functions in multiple hosts. In the case of genomic DNA, this may contain its own promoter or other regulatory elements (optionally in combination with a heterologous enhancer, such as the 35S enhancer discussed in the Examples below). The advantage of using a native promoter is that this may avoid pleiotropic responses. In the case of cDNA this may be under the control of an appropriate promoter or other regulatory elements for expression in the host cell

A promoter is a sequence of nucleotides from which transcription may be initiated of DNA operably linked downstream (i.e. in the 3′ direction on the sense strand of double-stranded DNA).

Operably linked means joined as part of the same nucleic acid molecule, suitably positioned and oriented for transcription to be initiated from the promoter. DNA operably linked to a promoter is “under transcriptional initiation regulation” of the promoter.

Suitable promoters include inducible promoters. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Thus nucleic acid described herein may be placed under the control of an externally inducible gene promoter to place expression (expressing the heterologous sequence) under the control of the user. An advantage of introduction of a heterologous gene into a plant cell, particularly when the cell is comprised in a plant, is the ability to place expression of the gene under the control of a promoter of choice, in order to be able to influence gene expression, and therefore QA or glycosylated QA biosynthesis, according to preference. Furthermore, mutants and derivatives of the wild-type gene, e.g. with higher or lower activity than wild-type, may be used in place of the endogenous gene.

Also provided is a gene construct, preferably a replicable vector, comprising a promoter (optionally inducible) operably linked to a biosynthetic gene described herein or a variant thereof.

Particularly of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed.) Oxford, BIOS Scientific Publishers, pp 121-148). Suitable vectors may include plant viral-derived vectors (see e.g. EP-A-194809).

Preferably the vectors which are for use in plants comprise border sequences which permit the transfer and integration of the expression cassette into the plant genome. Preferably the construct is a plant binary vector. Preferably the binary transformation vector is based on pPZP (Hajdukiewicz, et al. 1994). Other example constructs include pBin19 (see Frisch, D. A., L. W. Harris-Haller, et al. (1995). “Complete Sequence of the binary vector Bin 19.” Plant Molecular Biology 27: 405-409).

Suitable promoters which operate in plants include the Cauliflower Mosaic Virus 35S (CaMV 35S). Other examples are disclosed at pg. 120 of Lindsey & Jones (1989) “Plant Biotechnology in Agriculture” Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate). Positive selection system such as that described by Haldrup et al. 1998 Plant molecular Biology 37, 287-296, may be used to make constructs that do not rely on antibiotics.

As explained above, a preferred vector is a ‘CPMV-HT’ vector as described in WO2009/087391. The Examples below demonstrate the use of these pEAQ-HT expression plasmids.

These vectors (typically binary vectors) for use in the present invention will typically comprise an expression cassette comprising:
(i) a promoter, operably linked to
(ii) an enhancer sequence derived from the RNA-2 genome segment of a bipartite RNA virus, in which a target initiation site in the RNA-2 genome segment has been mutated;
(iii) a nucleic acid sequence as described above;
(iv) a terminator sequence; and optionally
(v) a 3′ UTR located upstream of said terminator sequence.

Enhancer sequences (or enhancer elements) are sequences derived from (or sharing homology with) the RNA-2 genome segment of a bipartite RNA virus, such as a comovirus, in which a target initiation site has been mutated. Such sequences can enhance downstream expression of a heterologous ORF to which they are attached. When present in transcribed RNA, such sequences may also enhance translation of a heterologous ORF to which they are attached.

A target initiation site is the initiation site (start codon) in a wild-type RNA-2 genome segment of a bipartite virus (e.g. a comovirus) from which the enhancer sequence in question is derived, which serves as the initiation site for the production (translation) of the longer of two carboxy coterminal proteins encoded by the wild-type RNA-2 genome segment.

Typically, the RNA virus will be a comovirus as described above.

Most preferred vectors are the pEAQ vectors of WO2009/087391 which permit direct cloning version by use of a polylinker between the 5′ leader and 3′ UTRs of an expression cassette including a translational enhancer of the invention, positioned on a T-DNA which also contains a suppressor of gene silencing and an NPTII cassettes.

The presence of a suppressor of gene silencing in such gene expression systems is preferred but not essential. Suppressors of gene silencing are known in the art and described in WO/2007/135480. They include HcPro from Potato virus Y, He-Pro from TEV, P19 from TBSV, rgsCam, B2 protein from FHV, the small coat protein of CPMV, and coat protein from TCV. A preferred suppressor when producing stable transgenic plants is the P19 suppressor incorporating a R43W mutation.

As described herein, a host may be converted from a phenotype whereby the host is unable to carry out an effective biosynthesis described herein to a phenotype whereby the host is able to carry out said biosynthesis, such that the product can be recovered therefrom or utilised in vivo to synthesize downstream products.

Biosynthesis may include (i) the conversion of OS to QA or to an intermediate such as oleanolic acid or echinocystic acid, (ii) the conversion of QA to QA-Tri, or to an intermediate such as QA-Mono or QA-Di (iii) the conversion of QA-Tri to QA-TriFRXX, or to an intermediate such as QA-TriF, QA-TriFR or QA-TriFX (iv) the conversion of QA into SpB or an intermediate, such as QA-TriF(Q)RXX.

Biosynthesis may also include (i) the conversion of OS into β-amyrin (ii) the conversion of β-amyrin to oleanolic acid (iii) the conversion of oleanolic acid to echinocystic acid (iv) the conversion of echinocystic acid to QA (v) the conversion of QA into 3-O-{[β-D-glucopyranosiduronic acid]oxy}-quillaic acid (“QA-GlcA”) (vi) the conversion of into 3-O-{[β-D-glucopyranosiduronic acid]oxy}-quillaic acid (“QA-GlcA”) into 3-O-{[β-D-galactopyranosyl-(1->2)]-β-D-glucopyranosiduronic acid}-quillaic acid (“QA-GlcA-Gal”) (vii) the conversion of 3-O-{[β-D-galactopyranosyl-(1->2)]-3-D-glucopyranosiduronic acid}-quillaic acid (“QA-GlcA-Gal”) into 3-O-{β-D-xylopyranosyl-(1->3)-[3-D-galactopyranosyl-(1->2)]-β-D-glucopyranosiduronic acid}-quillaic acid (“QA-GlcA-Gal-Xyl” or “QA-Tri”) (viii) the conversion of QA-Tri into 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF) (ix) the conversion of QA-TriF into 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR) (x) the conversion of QA-TriFR into 3-O-{β-D-xylopyranosyl-(1->3)-[3-D-galactopyranosyl-(1->2)]-3-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX); (xi) the conversion of QA-TriFRX into 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX); (xii) the conversion of QA-TriFRXX into 3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[β-D-quinovopyranosyl-(1→4)]-β-D-fucopyranosyl ester}-quillaic acid (QA-TriF(Q)RXX) and/or (xii) the conversion of QA-TriF(Q)RXX to 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-2)-[β-D-4-O-acetylquinovopyranosyl-(1→4)]-3-D-fucopyranosyl ester}-quillaic acid (SpB).

As explained above, triterpenoid biosynthetic genes described herein may also be engineered into plants. Suitable techniques are available in the art (see for example WO 2019/122259). 2,3-oxidosqualene is ubiquitous in higher plants due to its role in sterol biosynthesis, so biosynthesis as described herein has wide applicability in plant hosts. Suitable plant hosts include any plant that is amenable to transformation with Agrobacterium spp. As discussed herein, additional activities may be employed when practising the methods described herein in microorganisms.

Examples of suitable hosts include plants such as Nicotiana benthamiana and microorganisms such as yeast. These are discussed in more detail below.

The invention may comprise transforming the host with heterologous nucleic acid as described above by introducing the biosynthetic nucleic acid into the host cell via a vector and causing or allowing recombination between the vector and the host cell genome to introduce a nucleic acid according to the present invention into the genome.

In another aspect of the invention, there is provided a host cell transformed with a heterologous nucleic acid which comprises a plurality of triterpenoid biosynthetic nucleotide sequences each of which encodes a polypeptide which in combination have a biosynthesis activity described herein, wherein expression of said nucleic acid imparts on the transformed host the ability to carry out the biosynthesis or improves said ability in the host.

The invention further encompasses a host cell transformed with triterpenoid biosynthetic nucleic acid or a vector as described above (e.g. comprising the biosynthesis modifying nucleotide sequences) especially a plant or a microbial cell. In the transgenic host cell (i.e. transgenic for the nucleic acid in question) the transgene may be on an extra-genomic vector or incorporated, preferably stably, into the genome. There may be more than one heterologous nucleotide sequence per haploid genome.

The methods and materials described herein can be used, inter alia, to generate stable crop-plants that accumulate the biosynthetic triterpenoid saponin or other product. Examples of plants include row crops such as sunflower, potato, canola, dry bean, field pea, flax, safflower, buckwheat, cotton, maize, soybeans, and sugar beets. Major crop-plants such as corn, wheat, oilseed rape and rice may also be preferred hosts.

Plants which include a plant cell according to the invention are also provided.

Also provided are methods comprising introduction of such a construct into a plant cell or a microbial (e.g. bacterial, yeast or fungal) cell and/or induction of expression of a construct within a plant cell, by application of a suitable stimulus e.g. an effective exogenous inducer.

As an alternative to microorganisms, cell suspension cultures of engineered glycosylated QA-producing plant species, including also the moss Physcomitrella patens, may be cultured in fermentation tanks (see e.g. Grotewold et al. (Engineering Secondary Metabolites in Maize Cells by Ectopic Expression of Transcription Factors, Plant Cell, 10, 721-740, 1998).

Also provided is a host cell containing a heterologous construct described above, especially a plant or a microbial cell.

The discussion of host cells above in relation to reconstitution of QA or glycosylated QA biosynthesis in heterologous organisms applies mutatis mutandis here.

Also provided is a method of transforming a plant cell involving introduction of a construct as described above into a plant cell and causing or allowing recombination between the vector and the plant cell genome to introduce a nucleic acid described herein into the genome.

The invention further encompasses a host cell transformed with nucleic acid or a vector described herein (e.g. comprising the triterpenoid biosynthetic nucleotide sequence) especially a plant or a microbial cell. In the transgenic plant cell (i.e. transgenic for the nucleic acid in question) the transgene may be on an extra-genomic vector or incorporated, preferably stably, into the genome. There may be more than one heterologous nucleotide sequence per haploid genome.

Yeast has seen extensive employment as a triterpene-producing host and is therefore potentially well adapted for QA and then glycosylated QA biosynthesis as described herein, for example the biosynthesis of triterpenoid saponins.

In some preferred embodiments, the host is a yeast. For such hosts, it may be desirable to introduce additional genes to improve the flux of QA, and hence QA or glycosylated QA production as described above. Examples may include one or more plant cytochrome P450 reductases (CPRs) to serve as the redox partner to the introduced P450s, as well as an HMGR. It may likewise be desirable to introduce additional genes to contribute other elements of the QA or improve QA glycosylation pathways. These may include enzymes providing UDP-sugar donors and the like (see e.g. Ohashi T, Hasegawa Y, Misaki R, Fujiyama K (2016) “Substrate preference of citrus naringenin rhamnosyl transferases and their application to flavonoid glycoside production in fission yeast” (2016). Applied Microbiology and Biotechnology. 100 (2): 687-696.); Oka T, Jigami Y. (2006). “Reconstruction of de novo pathway for synthesis of UDP-glucuronic acid and UDP-xylose from intrinsic UDP-glucose in Saccharomyces cerevisiae”. FEBS J.273(12): 2645-57). In the light of the present disclosure, those skilled in the art can provide such ancillary activities as required.

Plants, which include a plant cell transformed as described above, are also provided.

If desired, following transformation of a plant cell, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

In addition to the regenerated plant, also provide are the following: a clone of such a plant, seed, selfed or hybrid progeny and descendants (e.g. F1 and F2 descendants). Also provided is a plant propagule from such plants, that is any part which may be used in reproduction or propagation, sexual or asexual, including cuttings, seed and so on. In all cases these plants or parts include the plant cell or heterologous biosynthesis modifying nucleic acid described above, for example as introduced into an ancestor plant.

It also provides any part of these plants (e.g. leaf, stem, dried or ground product, edible portion etc.), which in all cases include the plant cell or heterologous triterpenoid biosynthetic DNA described above.

The present invention also encompasses the expression product of any of the coding triterpenoid biosynthetic nucleic acid sequences disclosed and methods of making the expression product by expression from encoding nucleic acid therefore under suitable conditions, which may be in suitable host cells.

As described below, plant backgrounds such as those above may be natural or transgenic e.g. for one or more other genes relating to biosynthesis of a triterpenoid, such as QA or glycosylated QA, or otherwise affecting that phenotype or trait.

In modifying the host phenotypes, the triterpenoid biosynthetic nucleic acids described herein may be used in combination with any other gene, such as transgenes affecting the rate or yield of biosynthesis of a triterpenoid, such as QA or glycosylated QA, or its modification, or any other phenotypic trait or desirable property.

By use of a combination of genes, plants or microorganisms (e.g. bacteria, yeasts or fungi) can be tailored to enhance production of desirable precursors or reduce undesirable metabolism.

A triterpenoid biosynthetic sequence described herein may be used In vitro or in vivo to catalyse its respective biological activity.

For example, a method of converting 2,3-oxidosqualene (OS) into β-amyrin may comprise contacting OS with a Saponaria officinalis β-amyrin synthase (SobAS) comprising the amino acid sequence of SEQ ID NO 8 or a variant thereof, such that said OS is converted into β-amyrin. Also provided is the use of a Saponaria officinalis β-amyrin synthase (SobAS) comprising the amino acid sequence of SEQ ID NO 8 or a variant thereof to convert OS into β-amyrin.

A method of oxidising β-amyrin at the C28 position to a carboxylic acid may comprise contacting β-amyrin with a SoC28 oxidase polypeptide comprising the amino acid sequence of SEQ ID NO 2 or a variant thereof, such that the C28 position of said β-amyrin is oxidised to a carboxylic acid to produce oleanolic acid. Also provided the use of a SoC28 oxidase polypeptide comprising the amino acid sequence of SEQ ID NO 2 or a variant thereof, to oxidise the C28 position of β-amyrin to a carboxylic acid.

A method of oxidising oleanolic acid at the C16 position to an alcohol to produce echinocystic acid may comprise contacting oleanolic acid with a SoC28C16 oxidase polypeptide comprising an amino acid sequence of SEQ ID NO: 4 or a variant thereof, such that the C16 position of said oleanolic acid is oxidised to an alcohol, thereby producing echinocystic acid. Also provided is the use of a SoC28C16 oxidase polypeptide comprising an amino acid sequence of SEQ ID NO: 4 or variant thereof to oxidise the C16 position of oleanolic acid to an alcohol to produce echinocystic acid.

A method of oxidising β-amyrin, at the C16 position to an alcohol and the C28 position to a carboxylic acid to produce echinocystic acid may comprise contacting β-amyrin with a SoC28C16 oxidase polypeptide comprising an amino acid sequence of SEQ ID NO: 4 or a variant thereof, such that the C16 position of said β-amyrin is oxidised to an alcohol and the C28 position to a carboxylic acid, thereby producing echinocystic acid. Also provided is the use of a SoC28C16 oxidase polypeptide comprising an amino acid sequence of SEQ ID NO: 4 or variant thereof to oxidise the C28 and C16 positions of β-amyrin to produce echinocystic acid.

A method of oxidising echinocystic acid at the C-23 position to an alcohol to produce quillaic acid (QA) may comprise contacting echinocystic acid with a SoC23 oxidase polypeptide comprising the amino acid sequence of SEQ ID NO: 6 or a variant thereof, such that the C-23 position of said echinocystic acid is oxidised to an aldehyde, thereby producing quillaic acid (QA). Also provided is the use of a SoC23 oxidase polypeptide comprising the amino acid sequence of SEQ ID NO: 6 or a variant thereof to oxidise the C23 position of β-amyrin or an oxidised derivative thereof to an aldehyde produce quillaic acid (QA).

A method of converting quillaic acid (QA) into 3-O-{β-D-glucopyranosiduronic acid}-quillaic acid (“QA-GlcA”) may comprise contacting QA with Saponaria officinalis QA 3-O glucuronosyl transferase (“SoCSL”) polypeptide comprising the amino acid sequence of SEQ ID NO: 10 or a variant thereof, such that said QA is converted into QA-GlcA. Also provided is the use of a SoCSL polypeptide comprising the amino acid sequence of SEQ ID NO: 10 or a variant thereof to convert QA into QA-GlcA.

A method of converting 3-O-{β-D-glucopyranosiduronic acid}-quillaic acid (“QA-GlcA”) into 3-O-{[β-D-galactopyranosyl-(1->2)]-β-D-glucopyranosiduronic acid}-quillaic acid (“QA-GlcA-Gal”) may comprise; contacting QA-GlcA with a Saponaria officinalis QA-GlcA galactosyl transferase (“SoC3Gal”) polypeptide comprising the amino acid sequence of SEQ ID NO: 12 or a variant thereof, such that said QA-GlcA is converted into QA-GlcA-Gal. Also provided is the use of a Saponaria officinalis QA-GlcA galactosyl transferase (“SoC3Gal”) polypeptide comprising the amino acid sequence of SEQ ID NO: 12 or a variant thereof, to convert QA-GlcA into QA-GlcA-Gal.

A method of converting 3-O-{[β-D-galactopyranosyl-(1->2)]-β-D-glucopyranosiduronic acid}-quillaic acid (“QA-GlcA-Gal”) into 3-O-{β-D-xylopyranosyl-(1->3)-[3-D-galactopyranosyl-(1->2)]-β-D-glucopyranosiduronic acid}-quillaic acid (“QA-GlcA-[Gal]-Xyl” or “QA-Tri”) may comprise contacting QA-GlcA with a Saponaria officinalis QA-GlcA-Gal xylosyl transferase (“SoC3Xyl”) polypeptide comprising the amino acid sequence of SEQ ID NO: 14 or a variant thereof, such that said QA-GlcA-Gal is converted into QA-Tri. Also provided is the use of a Saponaria officinalis QA-GlcA-Gal xylosyl transferase (“SoC3Xyl”) polypeptide comprising the amino acid sequence of SEQ ID NO: 14 or a variant thereof, to convert QA-GlcA-Gal into QA-Tri.

A method of converting QA-Tri into 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF)) may comprise contacting QA-Tri with a Saponaria officinalis QA-Tri fucosyl transferase (“SoC28Fu”) polypeptide comprising the amino acid sequence of SEQ ID NO: 16 or a variant thereof, such that said QA-Tri is converted into QA-TriF. Also provided the use of a Saponaria officinalis QA-Tri fucosyl transferase (“SoC28Fu”) polypeptide comprising the amino acid sequence of SEQ ID NO: 16 or a variant thereof, to convert QA-Tri into QA-TriF.

A method of converting QA-TriF into 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR) may comprise contacting QA-TriF with a Saponaria officinalis QA-TriF rhamnosyl transferase

(“SoC28Rha”) polypeptide comprising the amino acid sequence of SEQ ID NO: 18 or a variant thereof, such that said QA-TriF is converted into QA-TriFR. Also provided is the use of a Saponaria officinalis QA-TriF rhamnosyl transferase (“SoC28Rha”) polypeptide polypeptide comprising the amino acid sequence of SEQ ID NO: 18 or a variant thereof to convert QA-TriF into QA-TriFR.

A method of converting QA-TriFR into 3-O-{β-D-xylopyranosyl-(1->3)-[3-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX) may comprise contacting QA-TriFR with a Saponaria officinalis QA-TriFR xylosyl transferase (“SoC28Xyl1”) polypeptide comprising the amino acid sequence of SEQ ID NO: 20 or a variant thereof, such that said QA-TriFR is converted into QA-TriFRX. Also provided is the use of a Saponaria officinalis QA-TriFR xylosyl transferase (“SoC28Xyl1”) polypeptide comprising the amino acid sequence of SEQ ID NO: 20 or a variant thereof to convert QA-TriFR into QA-TriFRX.

A method of converting QA-TriFRX into 3-O-{β-D-xylopyranosyl-(1->3)-[β-D-galactopyranosyl-(1->2)]-3-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX) may comprise contacting QA-TriFRX with a Saponaria officinalis QA-TriFRX xylosyl transferase (“SoC28Xyl2”) polypeptide comprising the amino acid sequence of SEQ ID NO: 22 or a variant thereof, such that said QA-TriFRX is converted into QA-TriFRXX. Also provided is the use of a Saponaria officinalis QA-TriFRX xylosyl transferase (“SoC28Xyl2”) comprising the amino acid sequence of SEQ ID NO: 22 or a variant thereof to convert QA-TriFRX into QA-TriFRXX.

A method of converting QA-TriFRXX into 3-O-{β-D-xylopyranosyl-(1→3)-[3-D-galactopyranosyl-(1→2)]-β-D-glucopyranosiduronic acid}-28-O-{β-D-xylopyranosyl-(1→3)-3-D-xylopyranosyl-(1→4)-α-L-rhamnopyranosyl-(1→2)-[β-D-quinovopyranosyl-(1->4)]-β-D-fucopyranosyl ester}-quillaic acid (QA-TriF(Q)RXX) may comprise contacting QA-TriFRXX with a Saponaria officinalis QA-TriFRXX quinovosyl transferase (“SoGH1”) polypeptide comprising the amino acid sequence of SEQ ID NO: 34 or a variant thereof, such that said QA-TriFRXX is converted into QA-TriF(Q)RXX. Also provided is the use of a Saponaria officinalis QA-TriFRXX quinovosyl transferase (“SoC28Xyl2”) comprising the amino acid sequence of SEQ ID NO: 34 or a variant thereof to convert QA-TriFRXX into QA-TriF(Q)RXX.

A method of converting QA-TriF(Q)RXX into 3-O-{β-D-xylopyranosyl-(1->3)-[3-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-2)-[3-D-4-O-acetylquinovopyranosyl-(1->4)]-β-D-fucopyranosyl ester}-quillaic acid (QA-TriF(Q-Ac)RXX) may comprise contacting QA-TriF(Q)RXX with a Saponaria officinalis QA-TriF(Q)RXX acetyl transferase (“SoBAHD1”) polypeptide comprising the amino acid sequence of SEQ ID NO: 36 or a variant thereof, such that said QA-TriF(Q)RXX is converted into QA-TriF(Q-Ac)RXX. Also provided is the use of a Saponaria officinalis QA-TriF(Q)RXX acetyl transferase (“SoBAHD1”) comprising the amino acid sequence of SEQ ID NO: 36 or a variant thereof to convert QA-TriF(Q)RXX into QA-TriF(Q-Ac)RXX (SpB).

In some embodiments, one or more of the nucleic acids or proteins described above may be used for the heterologous reconstitution of a biosynthetic pathway. Biosynthetic pathways are described above and may include one or more of the conversion of OS to QA, the conversion of QA to QA-Tri, the conversion of QA-Tri to QA-TriFRXX and the conversion of QA-TriFRXX into QA-TriF(Q-Ac)RXX.

Also further provided is a method of influencing or affecting biosynthesis in a host, such as a plant, the method comprising causing or allowing transcription of a heterologous triterpenoid biosynthetic nucleic acid as discussed above within the cells of the plant. The step may be preceded by the earlier step of introduction of the nucleic acid into a cell of the plant or an ancestor thereof. Biosynthesis may include the production of QA; a glycosylated QA, such as QA-Tri, QA-TriFRXX or QA-TriF(Q-Ac)RXX; or an intermediate of any one of these.

Such methods will usually form a part of, possibly one step in, a method of producing a glycosylated QA (e.g. QA-Tri, QA-TriFRXX or QA-TriF(Q-Ac)RXX) in a host such as a plant. Preferably, the method will employ a triterpenoid biosynthetic polypeptide or a variant thereof, as described above, or nucleic acid encoding either.

The methods described above may be used to generate QA or a glycosylated QA, such as QA-Tri, QA-TriFRXX or QA-TriF(Q-Ac)RXX, in a heterologous host, or may be used to generate an intermediate. The glycosylated QA will generally be non-naturally occurring in the species into which they are introduced.

Triterpenoids, including glycosylated forms of QA, such as QA-Tri, QA-TriFRXX or QA-TriF(Q-Ac)RXX, from the plants or methods described herein may be isolated and commercially exploited.

The methods above may form a part of, possibly one step in, a method of producing downstream products, such as QS-21 in a host. The method may comprise the steps of culturing the host (where it is a microorganism) or growing the host (where it is a plant) and then harvesting it and purifying the triterpenoid, for example a glycosylated QA, such as QA-Tri, QA-TriFRXX, or QA-TriF(Q-Ac)RXX or a downstream product or derivative (e.g. QS-21) product therefrom. The product thus produced forms a further aspect of the present invention. The utility of QS-21 is described above.

Alternatively, glycosylated QA, such as QA-Tri, QA-TriFRXX, QA-TriF(Q-Ac)RXX, may be recovered to allow for further chemical synthesis of downstream compounds.

The methods described herein embrace both the in vitro and in vivo production, or manipulation, of triterpenoids, such as QA and/or one or more glycosylated QAs. For example, triterpenoid biosynthetic polypeptides may be employed in fermentation via expression in microorganisms such as e.g. E. coli, yeast and filamentous fungi and so on. In some embodiments, one or more newly characterised triterpenoid biosynthetic sequences described herein may be used in these organisms in conjunction with one or more other biosynthetic genes.

In vivo methods are described extensively above, and generally involve the step of causing or allowing the transcription of, and then translation from, a recombinant nucleic acid molecule encoding the triterpenoid biosynthetic polypeptides.

In other embodiments, the triterpenoid biosynthetic polypeptides (enzymes) may be used in vitro, for example in isolated, purified, or semi-purified form. Optionally they may be the product of expression of a recombinant nucleic acid molecule.

Down-regulation of genes in a host may be desired e.g. to reduce undesirable metabolism or fluxes which might impact on yield of triterpenoids, such as QA or glycosylated QA. Such down regulation may be achieved by methods known in the art, for example using anti-sense technology.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Rothstein et al, 1987; Smith et al, (1988) Nature 334, 724-726; Zhang et al, (1992) The Plant Cell 4, 1575-1588, English et al., (1996) The Plant Cell 8, 179-188. Antisense technology is also reviewed in Bourque, (1995), Plant Science 105, 125-149, and Flavell, (1994) PNAS USA 91, 3490-3496.

An alternative to anti-sense is to use a copy of all or part of the target gene inserted in sense, that is the same, orientation as the target gene, to achieve reduction in expression of the target gene by co-suppression. See, for example, van der Krol et al., (1990) The Plant Cell 2, 291-299; Napoli et al., (1990) The Plant Cell 2, 279-289; Zhang et al., (1992) The Plant Cell 4, 1575-1588, and US-A-5,231,020. Further refinements of the gene silencing or co-suppression technology may be found in WO95/34668 (Biosource); Angell & Baulcombe (1997) The EMBO Journal 16, 12:3675-3684; and Voinnet & Baulcombe (1997) Nature 389: pg 553.

Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi) (See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245).

RNA interference is a two-step process. First, dsRNA is cleaved within the cell to yield short interfering RNAs (siRNAs) of about 21-23nt length with 5′ terminal phosphate and 3′ short overhangs (˜2nt). The siRNAs target the corresponding mRNA sequence specifically for destruction (Zamore P. D. Nature Structural Biology, 8, 9, 746-750, (2001)

Another methodology known in the art for down-regulation of target sequences is the use of “microRNA” (miRNA) e.g. as described by Schwab et al 2006, Plant Cell 18, 1121-1133. This technology employs artificial miRNAs, which may be encoded by stem loop precursors incorporating suitable oligonucleotide sequences, which sequences can be generated using well defined rules in the light of the disclosure herein.

In some embodiments, a method for influencing or affecting QA or glycosylated QA biosynthesis in a host, which method comprises any of the following steps of:
(i) causing or allowing transcription from a nucleic acid comprising the complement sequence of a host nucleotide sequence described herein, such that respective encoded polypeptide activity is reduced by an antisense mechanism;
(ii) causing or allowing transcription from a nucleic acid encoding a stem loop precursor comprising 20-25 nucleotides, optionally including one or more mismatches, of a host nucleotide sequence such that the respective encoded polypeptide activity is reduced by an miRNA mechanism;
(iii) causing or allowing transcription from nucleic acid encoding double stranded RNA corresponding to 20-25 nucleotides, optionally including one or more mismatches, of a host nucleotide sequence such that the respective encoded polypeptide activity is reduced by an siRNA mechanism.

It will be understood by those skilled in the art, in the light of the present disclosure, that additional genes may be utilised in the practice of the invention, to provide additional activities and/or improve expression or activity. These include those expressing co-factor or helper proteins, or other factors.

It will be appreciated that where these generic terms are used in relation to any aspect or embodiment, the meaning or disclosure will be taken to apply mutatis mutandis to any of these sequences individually.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### Abbreviations

QA-GlcA-[Gal]-Xyl or QA-Tri—3-O-{β-D-xylopyranosyl-(1->3)-[β-D-galactopyranosyl-(1->2)]-β-D-glucopyranosiduronic acid}-quillaic acid

QA-GlcA-Gal or QA-Di—3-O-{[β-D-galactopyranosyl-(1->2)]-β-D-glucopyranosiduronic acid}-quillaic acid

QA-GlcA or QA-Mono—3-O-{β-D-glucopyranosiduronic acid}-quillaic acid

QA-TriF—3-O-{β-D-xylopyranosyl-(1->3)-[β-D-galactopyranosyl-(1->2)]-3-D-glucopyranosiduronic acid}-28-O-{β-D-fucopyranosyl ester}-quillaic acid

QA-TriFR—3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid

QA-TriFRX—3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid

QA-TriFRXX—3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid

QA-TriF(Q)RXX—3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[3-D-quinovopyranosyl-(1→4)]-3-D-fucopyranosyl ester}-quillaic acid

QA-TriF(Q-Ac)RXX or SpB or Saponarioside B—3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-2)-[β-D-4-O-acetylquinovopyranosyl-(1→4)]-3-D-fucopyranosyl ester}-quillaic acid

QA—Quillaic acid

OS—2,3-oxidosqualene

Gal—D-Galactopyranose

GlcA—D-Glucopyranuronic acid (Additional numbers denote specific carbons i.e. GlcA-1)

Xyl—D-Xylopyranose

Rha—L-Rhamnopyranose

Ac—acetyl group

Qui—D-Quinovose (or Q)

SobAS or SobAS1—S officinalis β-amyrin synthase

SoC28 oxidase or SoC28 or CYP716A378—S officinalis quillaic acid C28 oxidase

SoC16 oxidase or SoC28C16 oxidase or SoC28C16 or CYP716A379—S officinalis quillaic acid C28 and C16 oxidase

SoC23 oxidase or SoC23 or CYP72A984—S officinalis quillaic acid C23 oxidase

SoQA-GlcAT or SoCSL or SoCSL1—S officinalis QA 3-O glucuronosyl transferase

SoQA-GalT or SoC3Gal or UGT73DL1—S officinalis QA-GlcA galactosyl transferase

SoQA-XyIT or SoC3Xyl or UGT3CC6—S officinalis QA-GlcA-Gal Xylosyl transferase

SoQA-TriFuT or SoC28Fu or UGT74CD1—S officinalis QA-Tri fucosyl transferase

SoFuSyn or SoSDR—S officinalis short chain dehydrogenase

SoQA-TriFRhaT or SoC28Rha or UGT79T1—S officinalis QA-TriF rhamnosyl transferase

SoQA-TriFRXyIT or SoC28Xyl1 or UGT79L3—S officinalis QA-TriFR xylosyl transferase

SoQA-TriFRXXyIT or SoC28Xyl2 or UGT73M2—S officinalis QA-TriFRX xylosyl transferase

SoGH1—S officinalis QA-TriFRXX quinovosyl transferase

SoBAHD1—S. officinalis QA-TriF(Q)RXX acetyl transferase tHMGR-Avena strigosa (diploid oat) truncated 3-hydroxy, 3-methylbutyryl-CoA reductase

### Experimental

### Materials and Methods

### RNA Synthesis and RNA-Seg Analysis

Total RNA was extracted from leaf and root of a representative soapwort plant using RNeasy Plant Mini kit (Qiagen) with a modified protocol described in [Mackenzie et al (1997) Plant Disease. 81: 222-226]. Along with RNA extraction, on-column DNase digestion was performed using RQ1 RNase-Free DNase (Promega). The cDNA used for amplification for putative soapwort bAS was generated from 0.8 μg of DNase treated RNA using GoScript™ Reverse Transcriptase (Promega) following manufacturer's instructions.

Total of 24 RNA samples were sent to the Earlham Institute (El) for transcriptome sequencing and RNA-seq analysis. NEBNext Ultra II Directional RNA-Seq library was constructed from 24 samples and were sequenced on two lanes of NovaSeq 6000 SP flow cell (150 pair-end reads). Transcriptome assembly was performed by El using Trinity de novo assembler (ver. 2.8.5), and ORF prediction and functional annotation was assigned using TransDecoder (ver. 5.5.0) and Human Readable Descriptions (AHRD, ver. 3.3.3), respectively. Transcript quantification was also provided by El using salmon (ver. 0.14.1).

### Identification of Candidate Genes

To identify candidate bAS in soapwort, the S. officinalis transcriptome was obtained from the 1,000 Plants (1KP) project (www.onekp.com) [Wicket et al (2014) PNAS 45 E4859-4868]]. A BLASTP search was performed against a translated S. officinalis protein database using previously characterized OSCs from other plant species listed in Table 1 as queries. The list of soapwort candidates was filtered by removing sequences with a length less than 500 amino acids (aa). The list was further filtered by performing phylogenetic analysis in MEGA-X (http://www.megasoftware.net). An amino acid alignment was made from putative soapwort genes and published OSCs from other plants listed in Table 1 using the MUSCLE algorithm (https://www.ebi.ac.uk/Tools/msa/muscle/). The alignment was used to create a phylogenetic tree using the neighbour-joining algorithm (Poisson model) with 1,000 bootstrap replicates. Based on the phylogenetic analysis, candidates that are unlikely to be bAS were removed from the list.

After identifying SobAS, all other pathway candidates were identified using the newly assembled S. officinalis transcriptome produced by El. Preliminary lists of candidate soapwort CYP450s, CSLs and UGTs were each created by performing BLASTP search using literature gene families as queries against the new soapwort transcriptome. The lists were filtered by removing candidates that were less than 500 aa in length. To further refine the lists, correlation analysis was performed to find candidates with similar expression pattern of SobAS. All bioinformatic analyses was performed in R. The transcript quantification results from salmon were read in using tximport (ver. 1.18.0). DEseq2 (ver. 1.30.1) was used to generate rlog library-normalized reads and were used for hierarchical clustering. Correlation analysis was performed using the Pearson's method.

cDNA Synthesis and Gateway® Cloning

For cloning of candidate soapwort genes, cDNA pool was generated from leaf and root RNA. First-strand cDNA synthesis was performed using GoScript Reverse transcription system (Promega) following manufacturer's protocol.

The coding sequences of candidate soapwort genes except SoGH1 were PCR amplified from the cDNA pool using gene specific primers with 5′ AttB sites. The coding sequence of SoGH1 with 5′AttB sites was synthesized by IDT. The PCR product was purified using QIAquick PCR Purification kit following manufacturer's protocol. Gateway® technology (Invitrogen) was used to transfer the purified PCR product into the entry vector and eventually into the expression vector. Briefly, BP recombination reaction was performed following manufacturer's instructions with equal ratios (˜150 ng each) of pDONR207 vector and the purified PCR product and were subsequently heat-shock transformed into chemically competent Escherichia coli cells (DH5a, ThermoFisher Scientific). Plasmids were recovered by performing plasmid preparations using QIAprep Spin Miniprep Kit (Qiagen) and were sequence verified. To generate expression clones, LR recombination reaction was performed following manufacturer's protocol with equal ratios (˜150 ng each) of the entry vector carrying the gene of interest and PEAQ-HT-DEST1 expression vector [Sainsbury et al (2009) Plant Biotechnol J 7(7): 682-693]. Plasmids were recovered again using QIAprep Spin Miniprep Kit (Qiagen) following manufacturer's protocol.

Transient Expression of Candidate Genes in N. benthamiana

Agrobacteria tumefaciens strain LBA4404 (Invitrogen) was used for transient expression of candidate genes in Nicotiana benthamiana. Agroinfiltration, sample harvest and preparation were performed as previously described in [Reed et al (2017) Metabolic Engineering, 42, 185-193].

GC-MS Analysis The GC-MS analysis was performed using an Agilent 7890B fitted with a Zebron AB5-HT Inferno Column (Phenomenex) using a 20-minute method program developed by James Reed (Osbourn laboratory). Briefly, 1 μL of each sample was injected into the inlet (250° C.) in pulse splitless mode (pulse pressure 30 psi). The oven temperature was held for 2 min at 170° C., then ramped to 300° C. at rate of 20° C./min and held at 300° C. for 11.5 mins for a total run time of 20 minutes. The mass spectrometry was performed using an Agilent 5977A Mass Selector Detector in scan mode from 60-800 m/z after a solvent delay of 8 mins. MassHunter workstation (Agilent) was used to analyse the resulting data.

### LC-MS Analysis

The LC-MS analysis was performed using a Shimadzu Prominence HPLC system fitted with IT-TOF mass spectrometer (Shimadzu) using aqueous formic acid (0.1% v/v) as solvent A and acetonitrile as solvent B.

The samples were analysed using a Kinetex XB-C18 100A (50×2.1 mm, 2.6 um; Phenomenex) column at a flow rate of 0.5 mL/min at 40° C., and an injection volume of 5 μL. The mass spectrometer was equipped with an electrospray in negative ionization mode (capillary temperature 250° C., nebulizing gas 1.3 min/L, heat block temperature 300° C., spray voltage −3.5 kV). The elution profile was as the following: 0-1 min, 5% B in A; 1-10 min, 55% B in A; 10-12 min, 100% B; 12-13 min, 100% B; 13-13.1 min, 5% B in A; 13.1-15.6 min, 5% B in A. MS/MS was used to monitor the daughter ion formation. LCMSolution software (Shimadzu) was used for data acquisition and processing. All authentic saponarioside pathway intermediate standards were provided by members of the Osbourn group.

S. officinalis Hairy Root Generation and Transformation

Seeds of S. officinalis were collected from the plants growing in JIC glasshouse. After washing with sterile water, seeds were kept in sterile water for 3-4 h and surface sterilized in sodium hypochlorite (5% w/v) for 30 min, followed by three times washing with sterile water. Further, seeds were washed for 1 min in 70% ethanol (v/v), followed by three times washing with sterile water. The seeds were germinated on MS

(Murashige and Skoog 1962) medium (pH 5.88), with 3% sucrose and 0.8% Agar. Sub-culturing of plantlets was done after 4 weeks and was maintained in MS medium (pH 5.88), with 3% sucrose and 0.8% Agar at 25° C. with 16 h light photoperiod. The hairy roots induction was performed with ATCC15834, which was found efficient (100% induction) among other tested strains (A4, A4RS, and LBA1334). Briefly, leaf explants were injected with respective bacterial solutions (100 uM Acetosyringone in MS, 1% sucrose, OD: 0.6) using needle with ˜5 injection per leaf explants. The infected explants were kept for 4 days in co-cultivation media comprised of semi-solid (0.8% Agar) MS medium supplemented with 3% sucrose and 100 uM acetosyringone in the dark for co-incubation at 25° C. Further, the explants were transferred to semi-solid (0.8% Agar) MS medium supplemented with 3% sucrose, 500 mg/l cefotaxime, and 50 mg/l Kanamycin for subsequent duration at 25° C., 16 h photoperiod, till removal of the bacteria and appearance of desired hairy roots.

Primers for silencing were designed from unique regions of S. officinalis β-amyrin synthase (SoβAS) and cloned in pDONR207 (Gateway-compatible vector). The subcloning was done in pK7WGIGW-2R, which offer dsRNA-mediated transgene silencing. For overexpression of SoBAS, the full-length sequence was cloned in pK7WG2R using gateway technology. The control hairy roots were raised using empty pK7WG2R (Zhao et al., 2016). All the constructs were transformed in ATCC15834 and after co-cultivation with wounded leaves, transgenic nature of hairy roots was assessed by dsRED fluorescence and PCR. Three weeks old dsRED expressing hairy roots grown on liquid B5 (with vitamins and sucrose) medium in dark were assessed for metabolite analysis.

### Results

### Identification and Characterization of SobAS Based on Phylogeny

The first committed step of triterpene biosynthesis is predicted to be the production of β-amyrin catalysed by an oxidosqualene cyclase (OSC), β-amyrin synthase (bAS). To identify candidate bASs in soapwort, we mined the translated S. officinalis transcriptome available from the 1,000 Plants (1KP) project (www.onekp.com; [Wickett et al supra]) and performed reciprocal BLASTP search using previously characterized OSCs from other plant species as search queries (Table 1). After phylogenetic analysis, SobAS was identified as a likely soapwort bAS candidate.

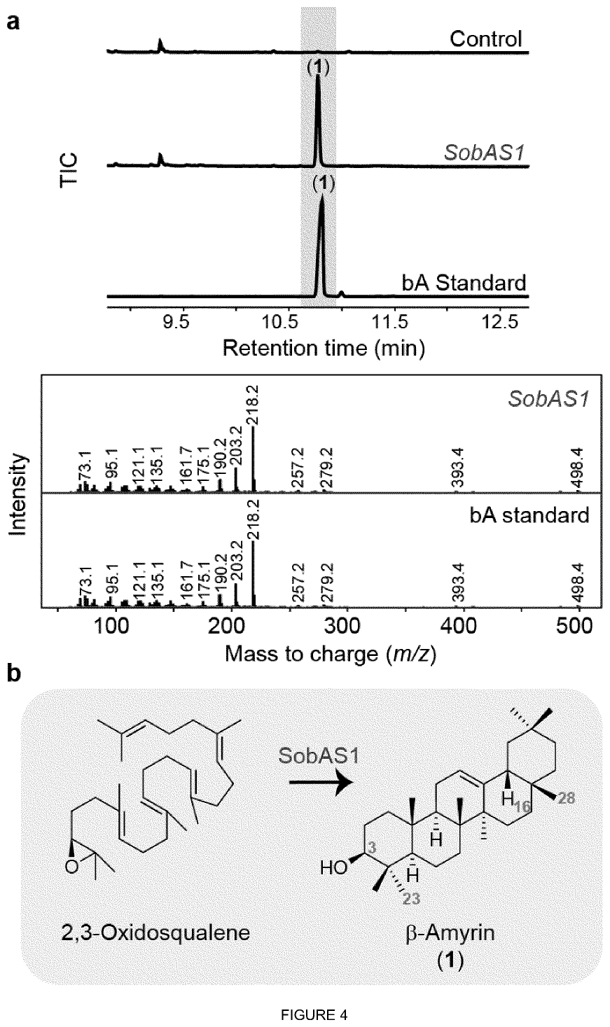

To test the activity of SobAS, we transiently expressed SobAS in Nicotiana benthamiana with the truncated HMG-CoA reductase (tHMGR) to increase the flux towards the MVA pathway [Reed et al 2017 supra]. The full open reading frames of SobAS and tHMGR were transformed into Agrobacterium tumefaciens and were co-infiltrated into leaves of N. benthamiana. The infiltrated leaves were harvested after 4 days post-infiltration, and the metabolites were extracted and analyzed using GC-MS. The transient expression of SobAS in N. benthamiana led to the formation of peak 1 with m/z 498, which corresponded to the commercial β-amyrin standard in both retention time and mass spectra (FIG. 4a). Peak 1 was not present in the leaves only expressing tHMGR which served as a negative control (FIG. 4a). Based on these results, the candidate SobAS is identified as an OSC capable of cyclizing oxidosqualene into β-amyrin (FIG. 4b).

### Identification of Saponarioside Pathway Genes by Co-Expression Analysis

As the publicly available soapwort transcriptome from the 1KP project lacks any organ specific transcriptome data, we performed RNA-seq analysis on six different soapwort organs (flower, flower bud, young leaf, old leaf, stem, root) differing in saponin content. The new soapwort transcriptome was used for further gene identification instead of the transcriptome available from the 1KP project.

Following the biosynthesis of β-amyrin, the next predicted step in saponarioside biosynthesis is the oxidation of β-amyrin to quillaic acid by three cytochrome P450s (CYP450s). To create a list of candidate soapwort CYP450s, BLASTP search was performed against the newly assembled soapwort transcriptome using literature CYP450s from the TriForC database (http://bioinformatics.psb.ugent.be/triforc/, [Miettinen et al (2017) Nature Comms 8 (1) 1-13]) as queries. This list was refined by removing any candidates less than 500 aa in length. To further refine the candidate list, Pearson co-expression analysis was performed with the expression pattern of SobAS characterized above. Any candidates with Pearson's correlation coefficient (PCC) less than 0.80 was filtered out from the candidate list (Table 3).

The next step in the saponarioside biosynthetic pathway is predicted to be the decoration of the quillaic acid by Family 1 UDP-dependent glycosyltransferases (UGTs). To identify candidate UGTs in soapwort, A list of previously characterized UGTs from other plant species was obtained from [Louveau et al (2019) Cold Spring Harbor Perspectives in Biology, 11(12), a034744] and was used as a BLASTP query against the S. officinalis transcriptome. The list of candidates was further refined similarly as above. Pearson co-expression analysis was performed using the expression profile of SobAS, and any candidates with PCC value less than 0.90 were filtered out of the list (Table 4).

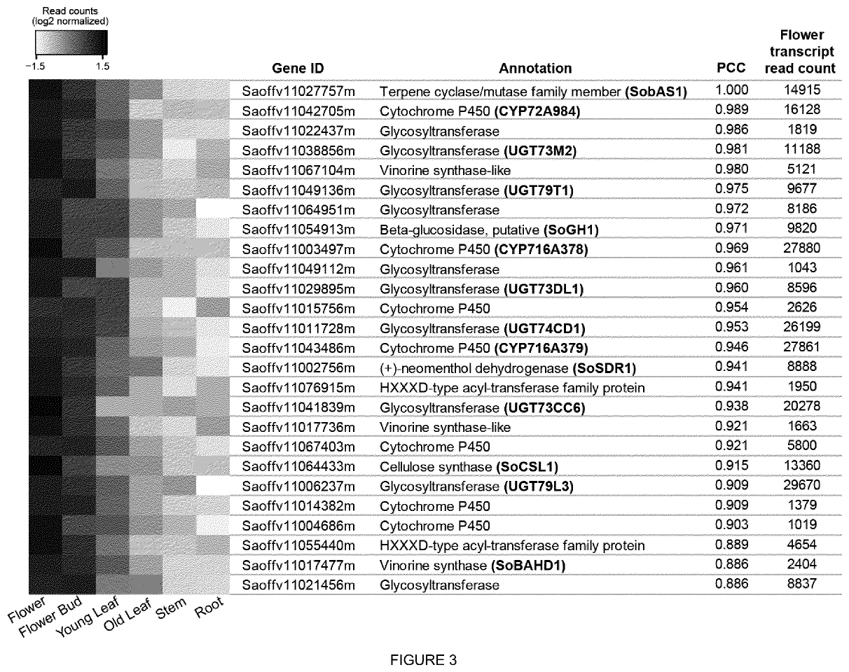

In addition to the UGTs, recent findings by Jozwiak and co-workers and the members of the Osbourn group have illustrated the ability of cellulose synthase like (CSL) genes to glucuronidate triterpene saponins (Jozwiak et al., 2020; WO/2020/260475). As such, we also searched for candidate CSLs in the soapwort transcriptome. A list of literature CSLs from other plant species was obtained from Reed et al., (In preparation) and used a BLASTP query against the soapwort transcriptome. The list of candidate soapwort CSLs was further refined by performing Pearson co-expression analysis using the expression profile of SobAS. Any candidate soapwort CSLs with PCC values less than 0.85 were filtered from the list (Table 5). The identified putative saponarioside biosynthetic genes all shared a similar expression profile along the different soapwort organs, suggesting their involvement in the same biosynthetic pathway (FIG. 3).

The list of candidates was further selected and refined based on high co-expression (PCC>0.88) with SobAS1 bait gene ranked using PCC, annotation and high absolute transcript count in the flower organ (FIG. 3).

Characterization of Candidate Genes by Transient Expression in N. benthamiana

Candidate saponarioside biosynthetic genes identified above were transiently expressed in N. benthamiana to test their activity. The open reading frames (ORFs) of candidate genes were either PCR amplified using primers listed in Table 2 or synthesized with upstream 5′ attb sites to allow for Gateway® cloning. The amplified or synthesized gene fragments were cloned into pDONR207 and were transferred into the plant expression vector pEAQ-HT-DEST1 [Sainsbury et al 2009 supra]. The expression constructs were individually transformed into Agrobacterium tumefaciens (LBA4404) for transient expression in N. benthamiana. In all experiments, A. tumefaciens strain carrying tHMGR was co-infiltrated to enhance the triterpene production in N. benthamiana. By screening the activity of top candidates in Tables 3-5 and FIG. 3, SoC28, SoC28C16, SoC23, SoCSL, SoC3Gal, SoC3Xyl, SoC28Fu, SoC28Rha, SoC28Xyl1, SoC28Xyl2, SoGH1 and SoBAHD1 were identified.

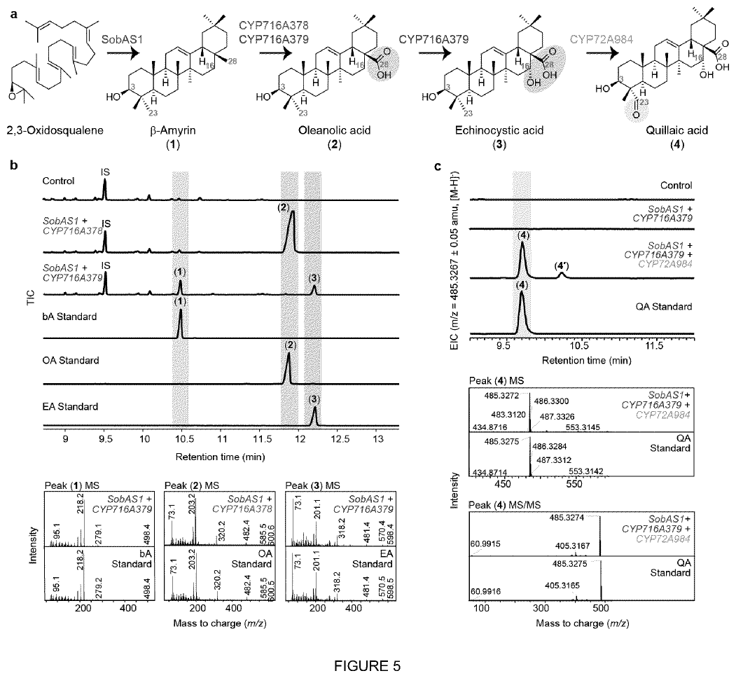

N. benthamiana leaves were co-infiltrated with A. tumefaciens strains each carrying ORFs of (i) tHMGR +SobAS+SoC28 or (ii) tHMGR+SobAS+SoC28C16 to test the activity of SoC28 and SoC28C16. The leaves were harvested 4 days after infiltration and the metabolites were extracted and analyzed using GC-MS. The co-expression of SobAS with SoC28 in N. benthamiana led to the formation of a peak 2 with m/z 585 (FIG. 5b). The retention time (RT), m/z and mass spectra of peak 2 present in the N. benthamiana extract corresponded with peak 2 found in the commercial oleanolic acid standard; therefore, peak 2 was identified as oleanolic acid (FIG. 5b). Interestingly, extracts from N. benthamiana leaves co-infiltrated with SobAS and SoC28C16 also produced oleanolic acid and an addition metabolite peak with m/z 570 (peak 3). The RT and mass spectra of peak 3 found in the N. benthamiana extract corresponded with peak 3 found in the echinocystic acid standard, thus peak 3 was identified as echinocystic acid. Both peaks 2 and 3 were not detected in the N. benthamiana leaves only expressing tHMGR used as a negative control. Based on these results, SoC28 is likely to be a CYP450 with a C28 oxidation activity, leading to the formation of oleanolic acid from β-amyrin, while SoC28C16 is likely to be a CYP450 with both C28 and C16 oxidation activity, leading to the production of both oleanolic acid and echinocystic acid.

Activity of SoC23 was tested by co-infiltrating N. benthamiana leaves with A. tumefaciens strains each carrying the OFRs of tHMGR+SobAS+SoC28C16+SoC23. The extracts of the harvested leaves were analyzed using HPLC-MS in negative ionization mode. The expression of SoC23 lead to the production of peak 4 with m/z 485.3 which corresponds to [M-H]⁻ of quillaic acid (FIG. 5b). Furthermore, the retention time and the mass spectra of peak 4 matched with the peak observed in the quillaic acid standard. Peak 4 was not detected in the negative control, where N. benthamiana leaves were only expressing tHMGR. Based on this result, SoC23 is likely to be a CYP450 with C-23 oxidation activity.

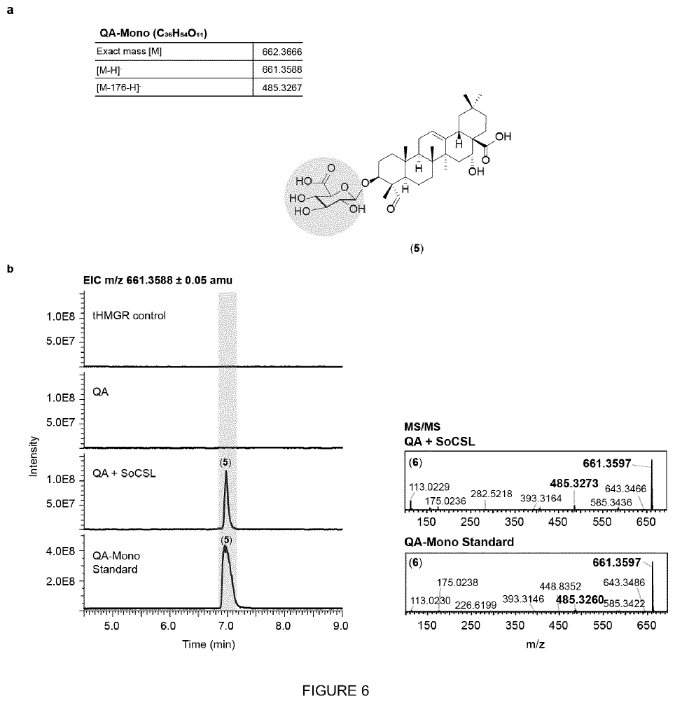

Following the biosynthesis of quillaic acid using genes from S. officinalis, candidate SoCSL was co-expressed with genes required to produce quillaic acid (tHMGR+SobAS+SoC28C16+SoC23). The extracts of the harvested leaves were analyzed using HPLC-MS in negative ionization mode. The HPLC-MS analysis revealed the production of peak 5 with m/z 661.3, the expected [M-H]⁻ of QA-Mono, by the addition of SoCSL (FIG. 6). Peak 5 is not detected in the negative control only expressing tHMGR, and the RT and mass spectra of peak 5 matches with the QA-Mono authentic standard from the Osbourn group (FIGS. 6). The MS/MS fragmentation pattern of peak 5 also shows the main fragment ion to be m/z 485.33, which corresponds to the expected [M-H]⁻ of quillaic acid. Based on the above results, peak 5 was identified as QA-Mono and SoCSL is a CSL able to glucuronidate quillaic acid.

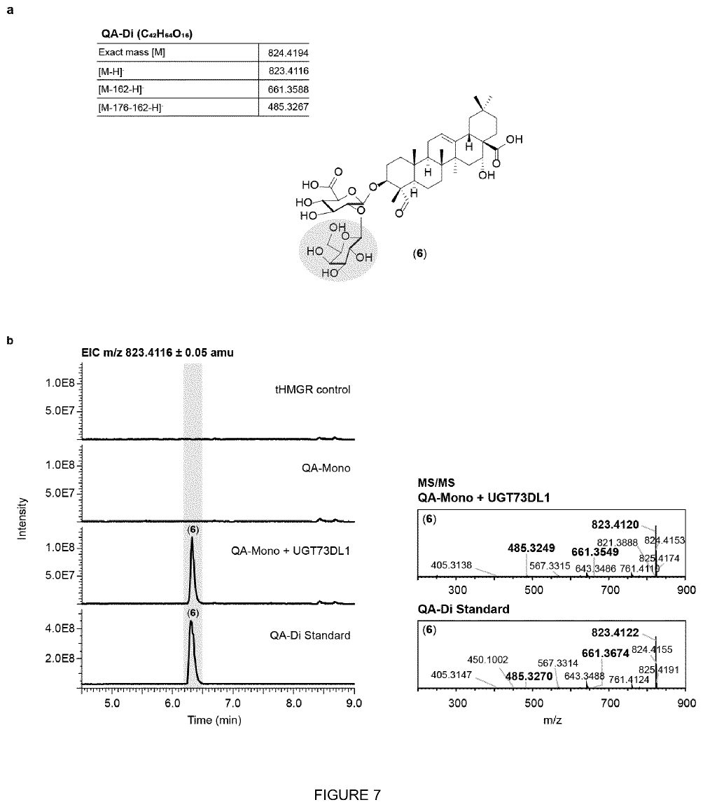

Next, the candidate SoC3Gal was co-expressed with genes required to produce quillaic acid (tHMGR+SobAS+SoC28C16+SoC23) and the newly characterized SoCSL. As similarly above, the harvested leaf extracts were analyzed using HPLC-MS. As a negative control, plant extracts expressing only genes producing quillaic acid and SoCSL was used. The addition of SoC3Gal resulted in the production of a new peak with m/z 823.4, which corresponds to the [M-H]⁻ of QA-Di (FIG. 7). Furthermore, the RT and mass-spectra of peak 6 produced by SoC3Gal matched with the peak produced by the authentic QA-Di standard. Additionally, the MS/MS fragmentation pattern revealed the major fragment ion of peak 6 to be m/z 485.32, corresponding to the [M-H]⁻ of quillaic acid, which suggests the fragmentation of the sugar chain from QA-Di (FIG. 7). Based on these results, peak 6 in FIG. 7 was putatively identified as QA-Di, and SoC3Gal to be a galactose-transferase from S. officinalis.

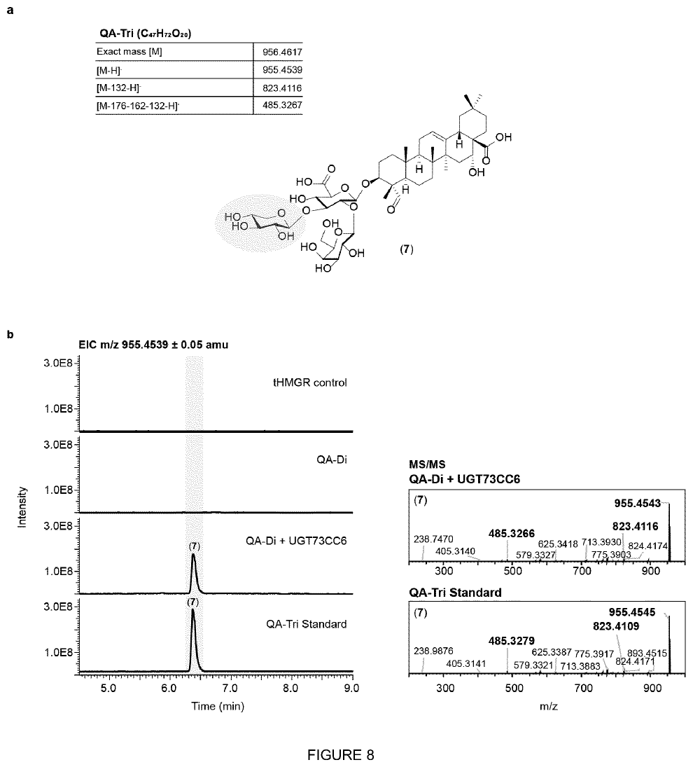

The candidate SoC3Xyl was characterized next. The genes required to produce QA-Di (tHMGR+SobAS+SoC28C16+SoC23+SoCSL+SoC3Gal) was co-expressed with the addition of SoC3Xyl in N. benthamiana. The harvested leaf extracts were analyzed using HPLC-MS for a new gene product with expected mass of m/z 955.4, corresponding to [M-H]⁻ of QA-Tri. While the negative control only co-expressing genes required to produce up to QA-Di did not produce any peak at the expected m/z, a new peak with m/z 955.4 was observed with the additional expression of SoC3Xyl (FIG. 8). Not only did peak 7 have the same RT and mass-spectra as the authentic QA-Tri standard, MS/MS fragmentation also revealed the major ions to be m/z 823.42 [M-H−Xyl]⁻ and m/z 485.33 [M-H−Xyl−Gal]⁻ (FIG. 8). Based on the results, peak 7 in FIG. 8 was putatively identified as QA-Tri and SoC3Xyl candidate to be xylose-transferase.

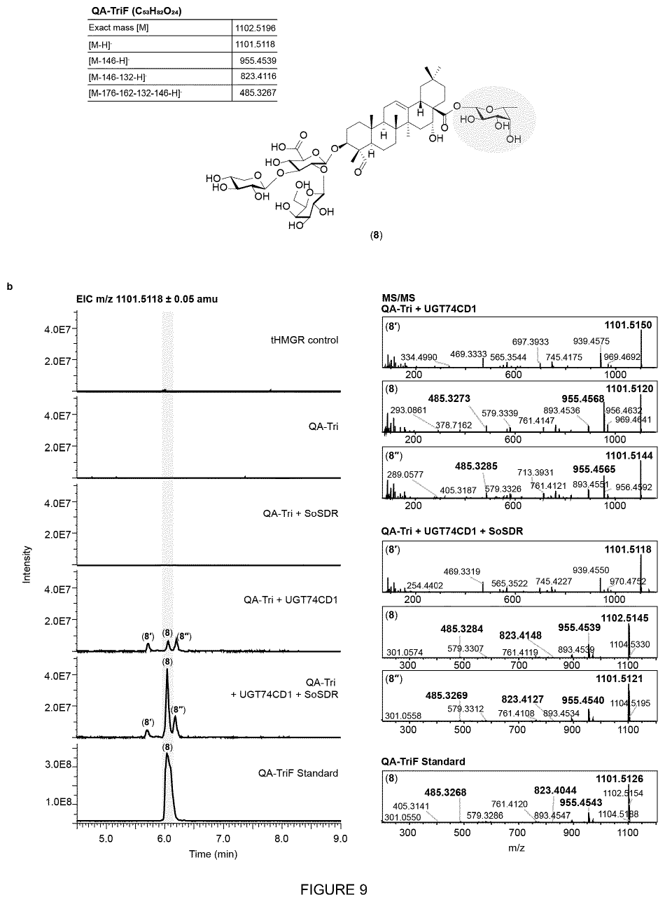

Our next focus was to characterize a sugar-transferase with the activity to transfer D-fucose to QA-Tri. Previous research by the Osbourn group has identified two genes, QsC28Fu and QsFuSyn, involved in the addition of D-fucose in QS-21 biosynthetic pathway. QsC28Fu was revealed to have UDP-4-keto-6-deoxy-glucose-transferase activity, while QsFuSyn was a 4-keto-reductase (Reed, Orme, El-Demerdash et al., 2023). In the process of this discovery, SoFuSyn was also identified and characterized to convert UPD-4-keto-6-deoxy-glucose to UPD-D-fucose. The SoC28Fu candidate gene was identified through co-expression analysis with SobAS, and we tested the activity of the candidate gene by transient expression in N. benthamiana. The combination of genes required to produce QA-Tri (tHMGR+SobAS+SoC28C16+SoC23+SoCSL+SoC3Gal+SoC3Xyl) with the addition of candidate SoC28Fu and previously characterized SoFuSyn was co-expressed in N. benthamiana. Following harvest, the leaves were extracted and analyzed on HPLC-MS. Peak 8 (m/z 1101.5) was produced by the additional activity of SoC28Fu, which corresponded in RT and mass-spectra as the peak produced by the authentic QA-TriF standard (FIG. 9). The peak was not detected in the negative control without SoFuC28 (FIG. 9). Furthermore, the MS/MS fragmentation pattern of peak 8 revealed the major daughter ions to be m/z 955.4, expected [M-H]⁻ of QA-Tri, and m/z 485.3, [M-H]⁻ of quillaic acid (FIG. 9). These results suggest that the candidate SoC28Fu, together with SoFuSyn, may transfer a fucose moiety to QA-Tri.

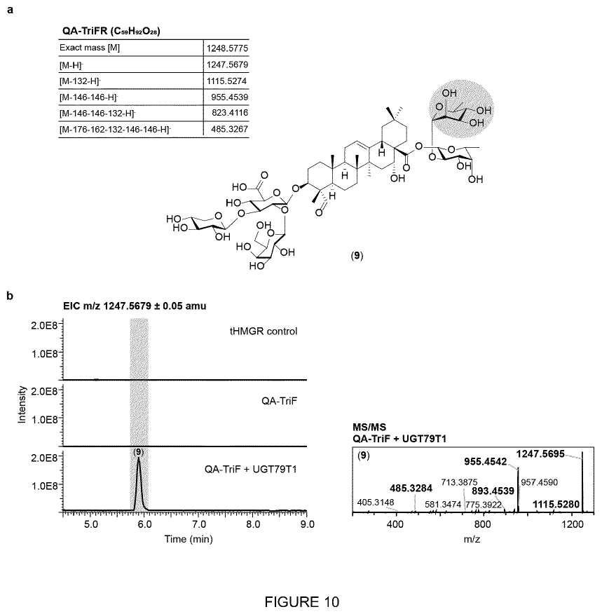

Next, the activity of candidate SoC28Rha was tested. The combination of genes required to produce QA-TriF (tHMGR+SobAS+SoC28C16+SoC23+SoCSL+SoC3Gal+SoC3Xyl+SoFuSyn+SoC28Fu) with the addition of SoC28Rha was co-expressed in N. benthamiana. The harvested leaf extracts were analyzed using HPLC-MS in negative ionization mode. The leaf extracts only expressing genes required to produce QA-TriF was used as a negative control. Peak 9 with the expected [M-H]⁻ of QA-TriFR, m/z 1247.5, was only detected in leaf extracts additionally expressing SoC28Rha (FIG. 10). Furthermore, the MS/MS fragmentation of peak 9 shows the major fragment ions to be m/z 955.4, corresponding to [M-H]⁻ of QA-Tri, and m/z 485.3, corresponding to [M-H]⁻ of quillaic acid, suggesting the fragment of the C28 sugar chain, followed by the C-3 sugar chain (FIG. 10). Based on these results, we putatively identified peak 9 in FIG. 10 to be QA-TriFR, and SoC28Rha to be a rhamnose-transferase.

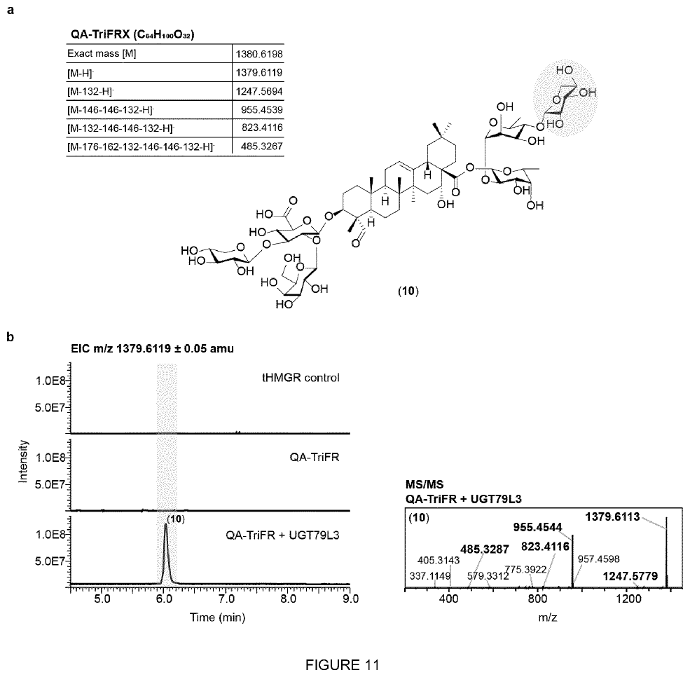

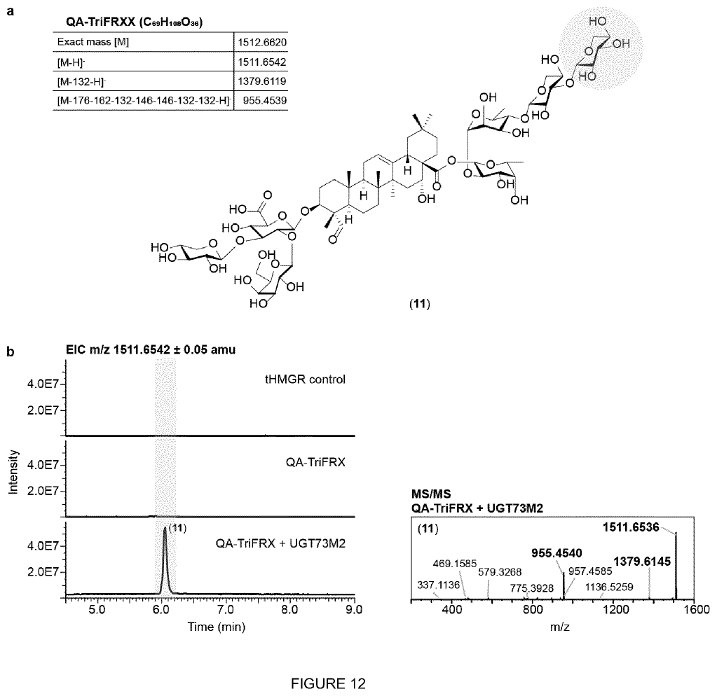

The next two enzymes that were characterized are SoC28Xyl1 and SoC28Xyl2. To test SoC28Xyl1, genes required to produce QA-TriFR (tHMGR+SobAS+SoC28C16+SoC23+SoCSL+SoC3Gal+SoC3Xyl+SoFuSyn+SoC28Fu+SoC28Rha) were co-expressed with the candidate SoC28Xyl1 in N. benthamiana. Extracts from leaf only expressing genes required to produce QA-TriFR were used as a negative control. Peak 10 with m/z 1379.6, the expected [M-H]⁻ of QA-TriFR, was only detected in samples expressing SoC28Rha with genes required to produce the substrate, QA-TriFR (FIG. 11). The MS/MS fragmentation reveals the major fragment ions of peak 10 to be m/z 955.4 and m/z 485.3, which suggests the loss of the C28 sugar chain to yield QA-Tri, followed by the loss of the C3 sugar chain, yielding quillaic acid (FIG. 11). The activity of SoC28Xyl2 was determined similarly to SoC28Xyl1. The genes required to produce QA-TriFRX (tHMGR+SobAS+SoC28C16+SoC23+SoCSL+SoC3Gal+SoC3Xyl+SoFuSyn+SoC28Fu+SoC28Rha+SoC28Xyl1) was co-expressed with the candidate SoC28Xyl2 in N. benthamiana. Tobacco leaves expressing genes required to produce QA-TriFRX without the addition of SoC28Xyl2 candidate was used as a negative control. The HPLC-MS analysis revealed that the production of peak 11 with the expected [M-H]⁻ of m/z 1511.6 was only observed in samples expressing SoC28Xyl2 (FIG. 12). Furthermore, the MS/MS analysis shows the major fragment ions of peak 11 to be m/z 1379.6 [M-H−X], m/z 955.4 [M-H−FRXX]⁻ and m/z 485.3 [M-H]⁻ of quillaic acid (FIG. 12). Overall, these results suggest SoC28Xyl1 and SoC28Xyl2 to be xylose-transferases in S. officinalis.

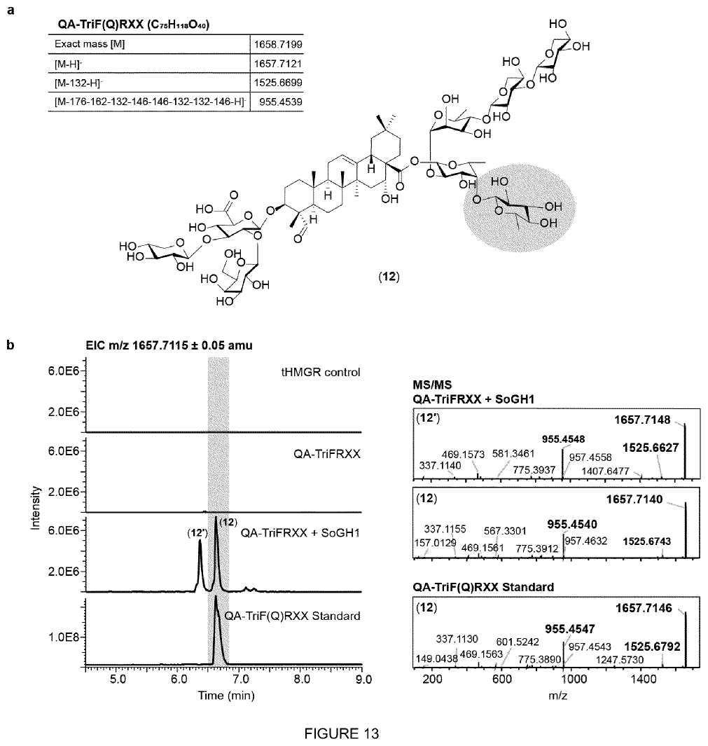

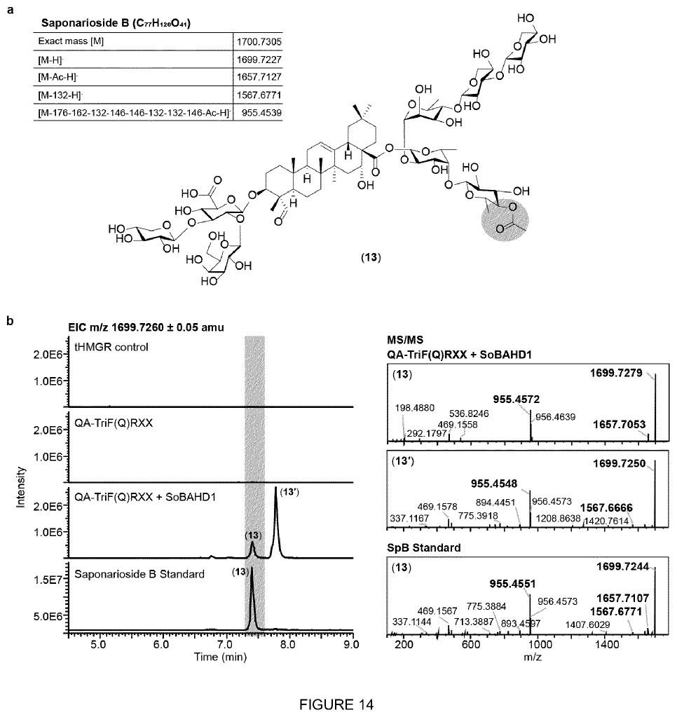

Thus far we have elucidated the genes and enzymes required for the biosynthesis of QA-TriFRXX (11). The steps responsible for the transfer of 4-O-acetylquinovose to 13 remains to be elucidated to complete the biosynthetic pathway to saponarioside B. Although GTs associated with plant natural product biosynthesis typically belong to family 1 of the GT superfamily, none of the UGTs in our main candidate list showed quinovosyltransferase activity towards 11. We therefore expanded our search for candidates by reviewing highly co-expressed genes with SobAS1 and noticed a glycosyl hydrolase family 1 (GH1) candidate exhibiting high level of co-expression (PCC =0.971) with SobAS1 (FIG. 3). We investigated the activity of SoGH1 against 11 using Agrobacterium-mediated transient expression in N. benthamiana. When SoGH1 was co-expressed with biosynthetic genes for 11, two new products (12′ and 12″) with different RTs but of the same mass ([M-H]⁻ =1657.7 m/z) corresponding to the anticipated mass of 11 plus deoxyhexose was observed (FIG. 13). In attempts to distinguish the two products, we performed tandem MS analysis on 12and 12′ which produced a same fragmentation pattern. The main fragment ions were 1525.7 m/z ([M-H]⁻ of QA-TriFRXX) and 955.4 m/z ([M-H]⁻ of QA-Tri), which suggested a loss of deoxyhexose, followed by the loss of the entire C-28 sugar chain, resulting in QA-Tri. We then compared 12 and 12′ with our authentic standard of 3-O-{β-D-xylopyranosyl-(1→3)-[β-D-galactopyranosyl-(1→2)]-3-D-glucopyranosiduronic acid}-28-O-{β-D-xylopyranosyl-(1→3)-β-D-xylopyranosyl-(1→4)-α-L-rhamnopyranosyl-(1→2)-[3-D-quinovopyranosyl-(1→4)]-β-D-fucopyranosyl ester}-quillaic acid (12, hereafter abbreviated as QA-TriF(Q)RXX) and observed that although the fragmentation of 12 and 12′ both matched with QA-TriF(Q)RXX, but only 12 had the same RT as QA-TriF(Q)RXX standard. Based on these results, SoGH1 may be involved in transfer of D-quinovse to QA-TriFRXX. With the successful pathway elucidation to 12, only an acetylation step remained to complete the biosynthetic pathway to SpB (13). We screened the functions of BAHD ATs in our main candidate list in FIG. 3 by transient expression in N. benthamiana leaves. LC-MS analysis of the resulting leaf extracts revealed that the co-expression of SoBAHD1, in combination with the gene set to produce 12,led to formation of two new products (13 and 13′) with the expected mass corresponding to SpB ([M-H]⁻=1699.7 m/z). Furthermore, tandem MS analysis revealed the same fragmentation pattern for both 13 and 13′. The major fragment ions were 1657.7 m/z ([M-H]⁻ of 12) and 955.5 m/z ([M]⁻ of 7), suggesting the fragmentation of an acetyl group, followed by the loss of the entire C-28 sugar chain (FIG. 14). However, only 13 produced by heterologous expression of SoBAHD1 corresponded in both RT and fragmentation pattern with authentic SpB standard. Based on these results, we identified 13 as SpB (13) produced by the acetylation of D-quinovose in 12 by SoBAHD1, and SoBAHD1 as an acetyltransferase with the ability to transfer an acetyl moiety to QA-TriF(Q)RXX to produce SpB.

The sequence similarity of saponarioside biosynthetic genes identified here and their counterparts in Q. saponaria involved in QS-21 biosynthesis was compared using amino acid sequences (Table 6). Although the first few genes showed high similarity in amino acid sequence, the rest of the pathway genes showed overall low sequence similarity. This suggests that the two pathways have likely established independently and suggests evidence for convergent evolution. The biosynthetic pathway of saponariosides that has been discussed here is illustrated in FIG. 15. However, the actual order of the biosynthesis can occur in any order in planta.

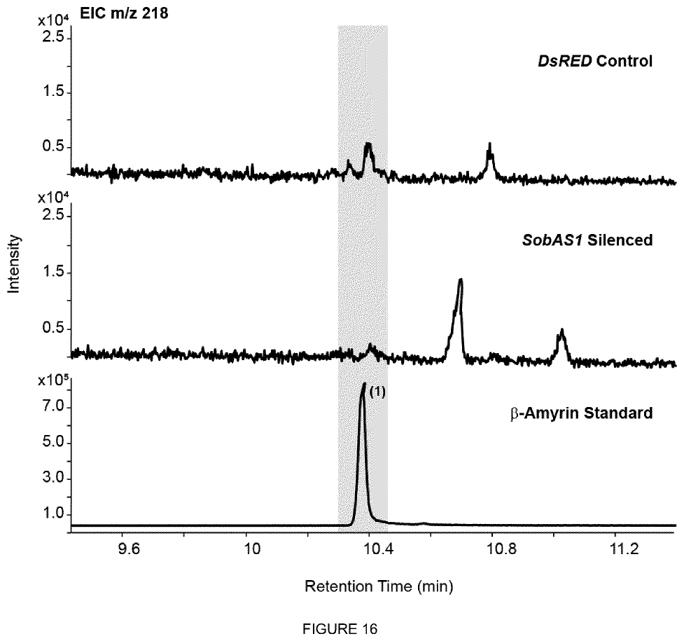

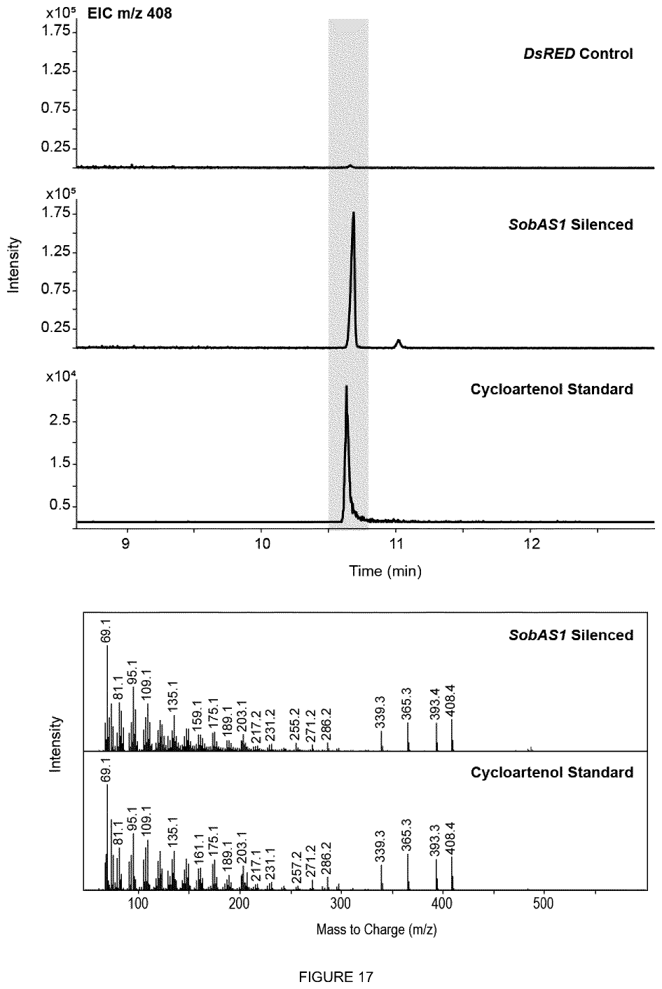

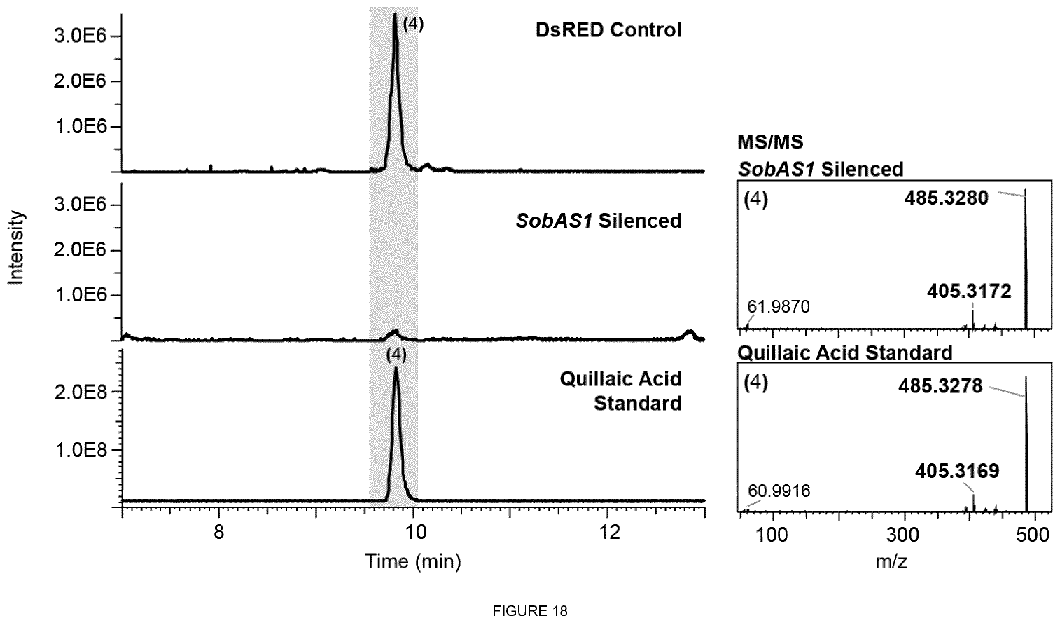

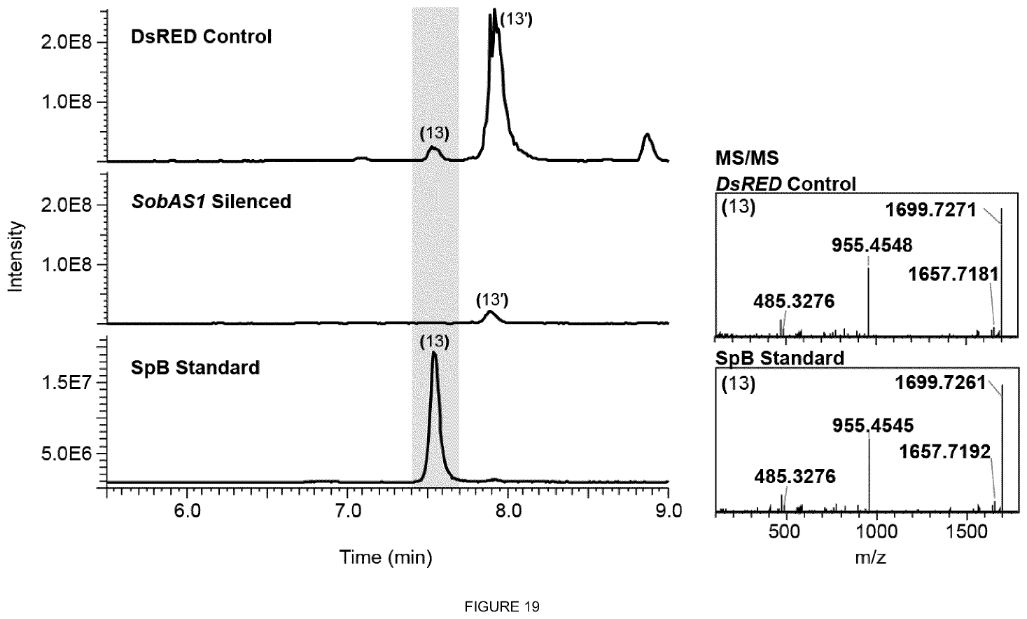

To investigate the role of the characterized genes in planta, hairy roots were successfully generated from soapwort seedlings. As a proof of concept, we silenced expression of SobAS1 in soapwort hairy roots and compared the metabolic profiles of the SobAS1 silenced hairy roots with DsRED expressing control hairy roots. Although β-amyrin was not detected in both the control and silenced hairy roots (FIG. 16), cycloartenol was accumulating in the SobAS1 silenced line only (FIG. 17). This may suggest that the silencing of SobAS1 in soapwort hairy roots resulted in the increase flux towards the sterol biosynthetic pathway. Further LC/MS analysis revealed that SobAS1 silenced hairy roots do not accumulate quillaic acid while abundant amount of quillaic acid is detected in the control hairy root line (FIG. 18). In agreement with this result, SpB is undetectable in SobAS1 silenced hairy roots while SpB is detected in the control roots (FIG. 19). Overall, these results show that SobAS1 is indeed an OSC responsible for β-amyrin biosynthesis in S. officinalis.

### Sequences

SEQ ID NO: 1
ATGGAACTCTTCTTCATATGTGGACTAGTACTCTTCTCCACCCTATCACTAATATCCCTCTTCCTCCTCCACAACCACAG
TTCTGCTCGGGGGTACAGGCTGCCCCCGGGCAGAATGGGATGGCCCTTCATAGGCGAGTCATACGAGTTTTTAGCAAACG
GGTGGAAAGGGTACCCGGAAAAGTTTATATTTAGCAGGTTGGCCAAGTATAAACCGAATCAAGTATTTAAGACGTCGATC
CTAGGAGAAAAAGTCGCGGTAATGTGTGGCGCGACATGTAACAAGTTCTTGTTCTCGAACGAGGGCAAATTAGTAAATGC
TTGGTGGCCGAATTCGGTTAATAAGATCTTCCCTTCTTCTACTCAAACTTCTTCCAAGGAAGAAGCTAAGAAGATGCGGA
AACTTCTCCCTACATTCTTTAAACCCGAGGCACTACAACGATACATACCCATCATGGACGAAATTGCGATCCGACACATG
GAGGACGAATGGGAAGGCAAATCCAAAATCGAAGTATTCCCACTCGCAAAACGCTACACATTTTGGCTAGCGTGCCGTCT
ATTCCTAAGCATAGACGACCCGGTACACGTAGCCAAATTCGCTGACCCGTTCAACGACATTGCCTCAGGGATCATATCGA
TCCCAATAGACCTCCCCGGCACACCATTCAACCGGGGAATTAAGGCCTCGAATGTCGTGAGACAGGAATTGAAGACCATA
ATAAAGCAGAGGAAATTGGACCTGTCCGACAACAAGGCGTCCCCGACACAGGATATATTGTCACACATGTTATTAACTCC
CGACGAAGACGGGCGGTATATGAATGAATTGGACATTGCTGATAAAATTCTCGGGTTGTTAATTGGAGGACATGATACTG
CAAGTGCTGCTTGTACTTTTGTTGTGAAGTTTCTTGCTGAACTCCCTCATATTTACGACGGTGTTTACAAAGAGCAAATG
GAGATAGCAAAGTCGAAAAAAGAAGGAGAGCGATTAAATTGGGAGGACATACAAAAGATGAAATATTCATGGAATGTGGC
CTGTGAAGTCATGCGTTTAGCACCTCCTCTTCAAGGCGCTTTTCGTGAAGCCCTCTCTGATTTTATGTACGCCGGTTTCC
AAATTCCCAAGGGTTGGAAGTTATATTGGAGCGCAAACTCAACACATAGGAACCCAGAATGCTTCCCAGAGCCGGAAAAA
TTCGACCCAGCAAGGTTCGATGGGAGCGGTCCGGCCCCATACACGTACGTACCGTTCGGAGGAGGGCCGAGAATGTGCCC
AGGAAAAGAGTATGCAAGGCTAGAAATATTGGTGTTCATGCACAACATTGTCAAGAGATTTAAGTGGGAAAAACTTATTC
CTGATGAAACCATTGTTGTTAATCCCATGCCGACCCCGGCTAAAGGCCTACCCGTCCGCCTTCGTCCTCATTCCAAACCC
GTAACTGTATCTGCTTAA
SoC28 oxidase (SoC28) nucleotide sequence
SEQ ID NO: 2
MELFFICGLVLFSTLSLISLFLLHNHSSARGYRLPPGRMGWPFIGESYEFLANGWKGYPEKFIFSRLAKYKPNQVFKTSI
LGEKVAVMCGATCNKFLFSNEGKLVNAWWPNSVNKIFPSSTQTSSKEEAKKMRKLLPTFFKPEALQRYIPIMDEIAIRHM
EDEWEGKSKIEVFPLAKRYTFWLACRLFLSIDDPVHVAKFADPENDIASGIISIPIDLPGTPFNRGIKASNVVRQELKTI
IKQRKLDLSDNKASPTQDILSHMLLTPDEDGRYMNELDIADKILGLLIGGHDTASAACTFVVKFLAELPHIYDGVYKEQM
EIAKSKKEGERLNWEDIQKMKYSWNVACEVMRLAPPLQGAFREALSDFMYAGFQIPKGWKLYWSANSTHRNPECFPEPEK
FDPARFDGSGPAPYTYVPFGGGPRMCPGKEYARLEILVFMHNIVKRFKWEKLIPDETIVVNPMPTPAKGLPVRLRPHSKP
VTVSA*
SoC28 oxidase (SoC28) amino acid sequence
SEQ ID NO: 3
ATGGAGCTAATTACCTTACTAAGTGCTCTTCTTGTTCTTGCTATAGTGAGTTTATCTACATTTTTCGTCCTTTACTATAA
TACTCCTACTAAGGACGGCAAAACTCTCCCTCCCGGTCGTATGGGCTGGCCTTTTATAGGCGAGTCCTACGACTTTTTTG
CCGCCGGTTGGAAAGGGAAGCCCGAGAGCTTCATTTTCGACCGGTTGAAGAAATTTGCTAAGGGGAACCTGAACGGTCAG
TTCAGGACGAGCTTGTTTGGGAACAAGTCGATTGTGGTGGCGGGGGCTGCTGCTAACAAGCTTCTTTTCTCGAATGAAAA
GAAGCTTGTTACCATGTGGTGGCCCCCGTCTATTGATAAGGCCTTCCCGTCGACTGCACAGTTGAGTGCGAACGAGGAGG
CCTTATTGATGAGGAAGTTTTTTCCTTCTTTTTTGATTAGAAGGGAGGCGCTCCAGCGCTACATCCCTATTATGGACGAC
TGCACCCGTCGTCACTTCGCGACGGGTGCGTGGGGTCCGTCGGACAAGATCGAGGCCTTCAATGTGACCCAAGACTACAC
GTTTTGGGTCGCCTGCAGAGTCTTCATGAGCATAGACGCTCAGGAAGACCCTGAGACGGTAGACTCCCTCTTTAGGCACT
TTAACGTGCTTAAAGCGGGAATCTACTCAATGCACATCGATCTCCCGTGGACGAACTTCCACCACGCGATGAAGGCGTCC
CACGCCATCAGGAGCGCCGTGGAGCAAATCGCGAAGAAAAGAAGGGCGGAATTGGCCGAGGGAAAGGCGTTCCCGACACA
AGATATGCTGTCTTACATGCTCGAAACGCCAATTACATCGGCGGAGGATAGCAAGGACGGGAAAGCGAAGTATTTGAATG
ACGCCGATATCGGGACGAAGATACTTGGTCTTCTTGTTGGTGGCCATGACACAAGTAGTACAGTTATTGCCTTCTTTTTC
AAGTTCATGGCTGAAAATCCTCATGTTTATGAGGCTATTTACAAAGAACAAATGGAGGTAGCGGCCACAAAAGCGCCGGG
GGAGCTTCTAAATTGGGATGACTTGCAGAAAATGAAGTACTCGTGGTGTGCGATTTGCGAGGTTATGCGTTTGACTCCCC
CTGTCCAAGGCGCCTTTCGCCAAGCCATCACCGACTTCACCCATAATGGTTACCTTATTCCCAAGGGTTGGAAGATATAC
TGGAGTACACACTCAACACACAGAAATCCCGAAATCTTCCCACAACCAGAGAAATTCGACCCAACAAGATTCGAAGGAAA
CGGGCCACCAGCGTTCTCATTCGTGCCATTCGGAGGAGGCCCGAGAATGTGTCCGGGTAAAGAATATGCAAGGCTACAAG
TGCTTACATTTGTGCACCACATTGTGACCAAATTCAAGTGGGAACAAATTCTACCTAATGAAAAGATCATTGTTAGCCCT
ATGCCGTACCCGGAGAAGAATCTTCCGCTTCGTATGATTGCTCGGTCTGAATCCGCCACCCTCGCTTAA
C28C16 oxidase (SoC28C16) nucleotide sequence
SEQ ID NO: 4
MELITLLSALLVLAIVSLSTFFVLYYNTPTKDGKTLPPGRMGWPFIGESYDFFAAGWKGKPESFIFDRLKKFAKGNINGQ
FRTSLFGNKSIVVAGAAANKLLFSNEKKLVTMWWPPSIDKAFPSTAQLSANEEALLMRKFFPSFLIRREALQRYIPIMDD
CTRRHFATGAWGPSDKIEAFNVTQDYTFWVACRVFMSIDAQEDPETVDSLFRHFNVLKAGIYSMHIDLPWTNFHHAMKAS
HAIRSAVEQIAKKRRAELAEGKAFPTQDMLSYMLETPITSAEDSKDGKAKYLNDADIGTKILGLLVGGHDTSSTVIAFFF
KFMAENPHVYEAIYKEQMEVAATKAPGELLNWDDLQKMKYSWCAICEVMRLTPPVQGAFRQAITDFTHNGYLIPKGWKIY
WSTHSTHRNPEIFPQPEKFDPTRFEGNGPPAFSFVPFGGGPRMCPGKEYARLQVLTFVHHIVTKFKWEQILPNEKIIVSP
MPYPEKNLPLRMIARSESATLA*
C28C16 oxidase (SoC28C16) amino acid sequence
SEQ ID NO: 5
ATGGAGTATTTGCCGTACATTGCAACATCAATTGCGTGCATAGTAATACTAAGATGGGCATTGAACATGATGCAATGGCT
ATGGTTCGAACCGAGGCGGTTGGAGAAATTACTTAGAAAACAAGGACTTCAAGGAAATTCATATAAGTTTTTATTTGGAG
ATATGAAGGAAAGTTCTATGTTGAGAAATGAAGCTTTAGCAAAGCCTATGCCTATGCCTTTTGATAATGACTACTTTCCT
CGTATTAATCCTTTTGTTGATCAACTTCTTAACAAATATGGTATGAATTGTTTCTTGTGGATGGGGCCTGTTCCGGCTAT
TCAAATCGGAGAACCAGAGTTAGTTAGGGAAGCTTTCAACCGGATGCACGAGTTTCAAAAGCCCAAAACTAACCCTTTGA
GTGCTTTACTCGCCACCGGACTTGTTAGCTACGAGGGCGACAAATGGGCCAAGCACCGCCGCCTTATCAACCCCTCTTTT
CATGTTGAAAAGCTCAAGCTTATGATTCCTGCATTCCGCGAGAGCATTGTGGAGGTGGTCAATCAATGGGAGAAGAAAGT
ACCTGAAAACGGCTCTGCTGAAATAGATGTATGGCCGTCTCTTACTAGTTTAACCGGAGATGTTATCTCAAGAGCTGCCT
TTGGCAGCGTGTATGGCGATGGAAGAAGGATTTTCGAACTTCTAGCTGTTCAGAAAGAACTCGTTTTAAGTCTGCTCAAG
TTTTCGTACATCCCTGGATACACGTATTTGCCAACAGAGGGAAACAAGAAGATGAAGGCGGTGAACAATGAGATACAAAG
ACTACTCGAAAACGTGATTCAAAACAGAAAGAAGGCGATGGAAGCCGGAGAAGCAGCAAAAGATGATCTGTTGGGTTTAC
TGATGGATTCCAATTACAAGGAGAGTATGCTTGAAGGCGGCGGGAAAAACAAAAAATTGATCATGAGTTTTCAAGATCTT
ATTGACGAGTGTAAGCTCTTCTTCTTAGCTGGGCACGAGACGACTGCTGTGTTACTTGTGTGGACTTTGATTTTGTTGTG
TAAGCACCAAGACTGGCAAACCAAAGCTCGCGAAGAAGTTTTGGCTACTTTTGGAATGTCGGAACCCACTGATTATGATG
CCTTAAACCGTCTCAAGATTGTGACAATGATACTAAATGAGGTCCTAAGATTGTACCCACCGGTTGTTTCAACCAACCGA
AAACTATTCAAGGGCGAAACAAAACTCGGAAACTTGGTAATACCACCAGGTGTCGGTATCTCACTATTAACCATCCAAGC
AAACCGTGACCCGAAAGTTTGGGGGGAGGATGCAAGTGAGTTCCGACCTGATAGATTTGCAGAAGGGCTAGTGAAGGCGA
CTAAGGGCAATGTCGCGTTTTTCCCCTTCGGTTGGGGTCCTAGGATTTGTATTGGCCAAAATTTTGCGCTGACCGAGTCA
AAGATGGCGGTTGCTATGATATTGCAACGCTTCACTTTCGACCTTTCACCGTCTTACACTCATGCTCCGTCGGGCCTTAT
TACTCTTAACCCGCAATATGGGGCTCCTCTCATGTTTCGTAGACGTTAA
SoC23 oxidase (SoC23) nucleotide sequence
SEQ ID NO: 6
MEYLPYIATSIACIVILRWALNMMQWLWFEPRRLEKLLRKQGLQGNSYKFLFGDMKESSMLRNEALAKPMPMPFDNDYFP
RINPFVDQLLNKYGMNCFLWMGPVPAIQIGEPELVREAFNRMHEFQKPKTNPLSALLATGLVSYEGDKWAKHRRLINPSF
HVEKLKLMIPAFRESIVEVVNQWEKKVPENGSAEIDVWPSLTSLTGDVISRAAFGSVYGDGRRIFELLAVQKELVLSLLK
FSYIPGYTYLPTEGNKKMKAVNNEIQRLLENVIQNRKKAMEAGEAAKDDLLGLLMDSNYKESMLEGGGKNKKLIMSFQDL
IDECKLFFLAGHETTAVLLVWTLILLCKHQDWQTKAREEVLATFGMSEPTDYDALNRLKIVTMILNEVLRLYPPVVSTNR
KLFKGETKLGNLVIPPGVGISLLTIQANRDPKVWGEDASEFRPDRFAEGLVKATKGNVAFFPFGWGPRICIGQNFALTES
KMAVAMILQRFTFDLSPSYTHAPSGLITLNPQYGAPLMFRRR*
SoC23 oxidase (SoC23) amino acid sequence
SEQ ID NO: 7
ATGTGGAGGTTAAAAATAGCAGAAGGTGGAAATGACCCGTATTTGTATAGCACAAACAATTTTGTAGGACGTCAAACTTG
GGAATTTGATAGCGAGTACGGTACTCCTGAAGCTATAAAAGAAGTAGAAGAAGCTCGACAAATTTTTTACAAAAATCGAT
TTCAAGTTAAGCCTTGTGGCGATCTTCTATGGCGTTTTCAGTTCCTAAGAGAGAAAAACTTCAAGCAAACAATACCGCAA
GTGAAGGTGGGTGATGGGGAGGAGGTCACCTACGAAGCCGCCTCAACGACGTTAAAGCGTTCCGTCAACTTACTCACGGC
CCTGCAGGCCGACGACGGTCACTGGCCTGCTGAAATTGCTGGCCCTCAATTTTTCCTCCCTCCTTTGGTGTTTTGCTTGT
ACATCACCGGACATCTCAACGTTGTTTTCAATGTTCATCACCGTGAAGAAATTCTTCGTAGCATTTATTATCACCAGAAT
GAGGATGGAGGGTGGGGGTTGCACATTGAAGGACACAGCACCATGTTCTGTACGGCGTTGAACTACATATGTTTGCGGAT
GCTAGGAGTCGGTCCTGATGAAGGAGACGACAACGCTTGCCCTAGGGCTCGTAAATGGATCCTCGACCATGGTAGTGTCA
CTCATATCCCTTCTTGGGGAAAGACTTGGCTTTCTATACTCGGTTTGTTTGATTGGTCCGGAAGTAACCCGATGCCACCT
GAGTTTTGGATTCTGCCTACTTTCATGCCTATGTATCCAGCGAAAATGTGGTGTTACTGTCGAATGGTGTACATGCCGAT
GTCGTACTTATACGGGAAGAGGTTCGTTGGTCCGATTACACCTCTAATCAAACAGCTCAGAGAGGAACTTTTCAGTGAAC
CGTTTGAAGAAATCAAGTGGAAAAAAGTCCGTCATCTGTGTGCACCGGAGGATCTCTACTACCCGCATCCATTGATTCAA
GACTTAATGTGGGACAGTCTTTACTTATTCACCGAGCCTCTTCTTACTCGCTGGCCGTTCAACAATTTGATACGACAGAA
GGCCTTACAAGTGACGATGGATCATATACATTACGAAGATGAGAACAGTCGATACATAACCATAGGATGCGTTGAAAAGG
TTTTGTGTATGTTGGCCTGTTGGGTTGAAGACCCAAATGGTGTTTGTTACAAAAAACATCTTGCTAGAGTTCCCGATTAT
ATATGGATTGCCGAGGATGGCCTTAAAATGCAGAGTTTTGGAAGTCAACAGTGGGACTGTGGCTTTGCTGTGCAAGCATT
ACTAGCTTCGAATATGAGTCTTGATGAAATCGGACCTGCCCTTAAGAAAGGCCACTTCTTTATCAAAGAGTCTCAGGTGA
AAGATAATCCCTCGGGTGATTTCAAGAGCATGCACCGTCATATCTCGAAGGGATCGTGGACGTTTTCTGACCAAGATCAT
GGTTGGCAGGTCTCTGACTGCACTGCAGAAGGCCTTAAGTGCTGCTTGATCTTATCAACCATGCCGCCAGAAATTGTTGG
AGAAAAGATGGACCCTGAGAGGCTCTACGACTCTGTCAATGTCCTGCTTTCTCTACAGAGTGAAAATGGAGGTCTATCTG
CTTGGGAACCAGCTGGAGCACAAGCTTGGTTAGAGCTTCTAAATCCAACGGAATTCTTCGCAGACATTGTGATCGAGCAT
GAGTATGTTGAATGTACTGGTGCATCAATTCAAGCTCTGGTATTATTCAAGAAAATGTACCCTGGTCACCGAAAGAAAGA
GATCGAAAATTTCATAGCCAAGGCCGCGAAATACCTCGAGGACACCCAATATCCAAACGGCTCTTGGTATGGAAATTGGG
GTGTGTGTTTCACGTATGGGACGTGGTTTGCGCTAGGAGGGCTAGCGGCAGCGGGCAAAACATACGCGAATTGTGCTGCG
ATGCGAAAAGGTGTTGAATTCCTTCTTAAGTCACAAAAGGAGGACGGTGGGTGGGGCGAAAGCTATGTTTCATGCCCGAA
AAAGGACTTCGTGCCGCTGGAAGGACCATCCAATCTAACTCAAACCGCATGGGCGTTGATGGGTCTAATTTACGCACGAC
AGATGGAGAGGGATCCGACACCGCTACACCAAGCAGCAAAGCTTTTGATCAATTCACAACTCGAAAACGGAGATTTCCCT
CAACAGGAAATAACAGGAGTATTCATGAAGAATTGCATGCTACACTATCCAATGTACAGGACTATTTATCCACTGTGGGC
TATTGCAGAATATAGGACGCATGTTCCTTTGAGGCTTAGTTAA
bAS (SobAS) nucleotide sequence
SEQ ID NO: 8
MWRLKIAEGGNDPYLYSTNNFVGRQTWEFDSEYGTPEAIKEVEEARQIFYKNRFQVKPCGDLLWRFQFLREKNFKQTIPQ
VKVGDGEEVTYEAASTTLKRSVNLLTALQADDGHWPAEIAGPQFFLPPLVFCLYITGHLNVVENVHHREEILRSIYYHQN
EDGGWGLHIEGHSTMFCTALNYICLRMLGVGPDEGDDNACPRARKWILDHGSVTHIPSWGKTWLSILGLFDWSGSNPMPP
EFWILPTFMPMYPAKMWCYCRMVYMPMSYLYGKRFVGPITPLIKQLREELFSEPFEEIKWKKVRHLCAPEDLYYPHPLIQ
DLMWDSLYLFTEPLLTRWPENNLIRQKALQVTMDHIHYEDENSRYITIGCVEKVLCMLACWVEDPNGVCYKKHLARVPDY
IWIAEDGLKMQSFGSQQWDCGFAVQALLASNMSLDEIGPALKKGHFFIKESQVKDNPSGDFKSMHRHISKGSWTFSDQDH
GWQVSDCTAEGLKCCLILSTMPPEIVGEKMDPERLYDSVNVLLSLQSENGGLSAWEPAGAQAWLELLNPTEFFADIVIEH
EYVECTGASIQALVLFKKMYPGHRKKEIENFIAKAAKYLEDTQYPNGSWYGNWGVCFTYGTWFALGGLAAAGKTYANCAA
MRKGVEFLLKSQKEDGGWGESYVSCPKKDFVPLEGPSNLTQTAWALMGLIYARQMERDPTPLHQAAKLLINSQLENGDEP
QQEITGVFMKNCMLHYPMYRTIYPLWAIAEYRTHVPLRLS*
bAS (SobAS) amino acid sequence
SEQ ID NO: 9
ATGTCACCCCACAACACCTGCACTCTACAAATAACCCGAGCCCTCCTCAGCCGCCTCCACATCCTCTTCCACTCCGCCCT
CGTCGCCTCCGTCTTCTACTACCGCTTTTCCAACTTCTCCTCTGGCCCGGCATGGGCCCTCATGACTTTCGCCGAGCTCA
CCCTCGCCTTCATCTGGGCCCTCACCCAGGCCTTCCGCTGGCGGCCCGTCGTCCGGGCCGTCTTCGGGCCCGAGGAGATT
GACCCGGCCCAGCTCCCGGGTCTGGACGTGTTCATATGCACGGCAGACCCGAGGAAGGAGCCGGTGATGGAGGTGATGAA
CTCGGTGGTGTCGGCATTGGCGTTGGATTATCCGGCAGAGAAGCTGGCGGTTTACTTGTCGGACGACGGCGGGTCGCCCT
TGACTAGGGAGGTTATTAGGGAGGCTGCCGTGTTTGGGAAGTACTGGGTCGGGTTTTGTGGGAAGTATAATGTTAAGACG
AGGTGTCCTGAGGCCTATTTTAGTTCGTTTTGTGATGGTGAAAGAGTTGATCATAATCAGGATTATTTGAACGACGAGCT
TTCCGTCAAGTCGAAATTTGAAGCGTTTAAGAAGTATGTGCAAAAAGCAAGTGAAGACGCCACCAAATGTATTGTTGTCA
ATGATCGTCCTTCTTGTGTTGAGATTATTCATGACAGCAAGCAGAACGGAGAGGGTGAAGTGAAAATGCCGCTTCTTGTT
TACGTAGCCAGGGAAAAAAGACCGGGTTTTAATCACCATGCTAAAGCCGGAGCCATTAATACACTTCTTCGAGTGTCGGG
TTTACTGAGCAATAGCCCTTTCTTTTTGGTGTTGGATTGTGATATGTACTGTAATGATCCAACGTCTGCGCGTCAAGCTA
TGTGCTTCCATCTTGACCCGAAACTAGCTCCCTCTCTCGCGTTTGTGCAATACCCTCAAATTTTCTACAACACCAGCAAA
AACGACATCTATGATGGTCAGGCCAGAGCAGCTTTTAAGACTAAATATCAAGGCATGGATGGTCTTAGAGGGCCGGTTAT
GAGTGGCACGGGGTATTTCTTGAAGAGGAAAGCATTGTACGGAAAACCACACGACCAAGATGAATTACTCAGGGAGCAGC
CAACGAAGGCCTTTGGCTCCTCTAAGATATTCATCGCGTCCCTTGGTGAAAATACCTGTGTTGCCTTGAAAGGATTGAGT
AAAGACGAGTTGTTGCAAGAGACTCAAAAATTGGCTGCTTGTACATACGAATCAAACACGTTATGGGGTAGCGAGGTTGG
ATACTCGTACGACTGCTTGTTGGAGAGCACATACTGTGGGTACTTATTACACTGCAAAGGATGGATCTCAGTATATCTAT
ACCCGAAAAAGCCGTGTTTCTTGGGGTGTGCAACAGTGGACATGAATGATGCCATGCTTCAGATAATGAAATGGACTTCT
GGATTGATTGGCGTTGGCATATCAAAGTTCAGCCCGTTCACATACGCCATGTCTCGGATCTCCATTATGCAAAGTCTTTG
CTATGCTTACTTCGCTTTTTCGGGCCTATTTGCTGTCTTCTTCTTGATCTATGGCGTTGTTCTTCCGTATTCCCTCTTGC
AGGGTGTTCCGCTCTTCCCCAAGGCAGGAGATCCATGGCTTTTGGCATTTGCGGGAGTATTCATATCCTCGCTTCTTCAG
CACCTGTACGAGGTTCTCTCAAGCGGAGAAACAGTGAAAGCGTGGTGGAACGAGCAAAGAATCTGGATCATAAAATCAAT
CACCGCCTGTCTGTTTGGTCTTCTGGACGCTATGCTTAACAAAATTGGCGTCTTAAAGGCTAGTTTCAGACTGACAAACA
AGGCTGTCGACAAACAAAAACTCGATAAATACGAGAAGGGCAGGTTCGATTTCCAAGGCGCACAAATGTTCATGGTCCCT
CTCATGATTCTGGTGGTATTCAATTTGGTCTCGTTCTTTGGCGGCTTAAGAAGAACCGTCATTCATAAAAACTACGAAGA
CATGTTCGCGCAGCTTTTCCTCTCGTTGTTCATTCTAGCTCTTAGCTATCCTATCATGGAGGAGATTGTCCGAAAAGCTA
GAAAAGGTCGCTCTTAA
SoQA-GICAT (SoCSL) nucleotide sequence
SEQ ID NO: 10
MSPHNTCTLQITRALLSRLHILFHSALVASVFYYRFSNFSSGPAWALMTFAELTLAFIWALTQAFRWRPVVRAVFGPEEI
DPAQLPGLDVFICTADPRKEPVMEVMNSVVSALALDYPAEKLAVYLSDDGGSPLTREVIREAAVFGKYWVGFCGKYNVKT
RCPEAYFSSFCDGERVDHNQDYLNDELSVKSKFEAFKKYVQKASEDATKCIVVNDRPSCVEIIHDSKQNGEGEVKMPLLV
YVAREKRPGFNHHAKAGAINTLLRVSGLLSNSPFFLVLDCDMYCNDPTSARQAMCFHLDPKLAPSLAFVQYPQIFYNTSK
NDIYDGQARAAFKTKYQGMDGLRGPVMSGTGYFLKRKALYGKPHDQDELLREQPTKAFGSSKIFIASLGENTCVALKGLS
KDELLQETQKLAACTYESNTLWGSEVGYSYDCLLESTYCGYLLHCKGWISVYLYPKKPCFLGCATVDMNDAMLQIMKWTS
GLIGVGISKFSPFTYAMSRISIMQSLCYAYFAFSGLFAVFFLIYGVVLPYSLLQGVPLFPKAGDPWLLAFAGVFISSLLQ
HLYEVLSSGETVKAWWNEQRIWIIKSITACLFGLLDAMLNKIGVLKASFRLTNKAVDKQKLDKYEKGRFDFQGAQMFMVP
LMILVVFNLVSFFGGLRRTVIHKNYEDMFAQLFLSLFILALSYPIMEEIVRKARKGRS*
SoQA-GIcAT (SoCSL) amino acid sequence
SEQ ID NO: 11
ATGGGTTCAAATACAGAAGCAACTGAAATACCCAAAATGCCCTTGAAAATAGTCTTCCTTACACTTCCTATAGCCGGACA
CATGCTCCACATTGTAGACACCGCAAGCACATTTGCCATACATGGAGTCGAGTGTACCATAATCACTACCCCTGCAAATG
TCCCTTTCATCGAAAAATCAATCTCTGCAACCAACACCACAATTCGACAGTTCCTCAGTATCCGCCTCGTCGATTTCCCC
CATGAAGCTGTCGGCCTTCCTCCCGGTGTCGAAAACTTCAGTGCAGTCACGTGTCCGGATATGAGACCCAAAATATCGAA
AGGACTTTCGATCATACAAAAACCAACTGAAGACTTAATCAAGGAAATATCACCTGATTGTATTGTTTCTGACATGTTTT
ACCCTTGGACTTCTGATTTCGCCCTTGAAATAGGTGTTCCAAGGGTGGTTTTTCGCGGTTGTGGGATGTTTCCCATGTGT
TGTTGGCATAGTATTAAGTCACATTTACCACATGAGAAGGTTGACAGAGATGATGAAATGATTGTTCTTCCTACATTGCC
TGATCATATAGAGATGAGAAAATCTACATTACCTGATTGGGTAAGGAAACCAACTGGGTACAGTTATTTGATGAAGATGA
TTGATGCGGCCGAATTGAAGAGTTATGGAGTAATTGTTAATAGTTTTAGTGATTTAGAGAGGGATTATGAGGAGTATTTT
AAGAATGTCACCGGGTTAAAGGTGTGGACCGTCGGTCCGATTTCGTTACATGTGGGTCGGAATGAGGAGTTAGAAGGGTC
AGATGAGTGGGTCAAATGGCTAGATGGGAAAAAACTAGACTCGGTTATTTATGTTAGTTTTGGTGGGGTGGCGAAGTTTC
CACCCCACCAGCTGAGAGAAATCGCGGCCGGATTAGAATCATCTGGCCACGATTTTGTTTGGGTGGTGAGGGCGAGTGAC
GAAAATGGCGACCAAGCTGAAGCGGATGAGTGGTCCCTACAAAAATTTAAAGAGAAAATGAAGAAAACTAACCATGGGTT
GGTTATAGAGAGTTGGGTCCCACAACTTATGTTTTTGGAACATAAGGCTATCGGAGGAATGTTGACACATGTTGGTTGGG
GTACAATGTTGGAAGGGATTACAGCGGGTTTACCGTTGGTGACGTGGCCATTGTATGCCGAGCAGTTTTACAATGAGAGG
TTGGTGGTTGATGTGTTGAAGATTGGAGTTGGTGTTGGGGTGAAAGAGTTCTGTGGGTTGGATGATATTGGCAAGAAGGA
GACCATTGGTAGGGAGAATATCGAGGCATCGGTGAGATTAGTGATGGGCGATGGCGAGGAGGCGGCTGCCATGAGACTGC
GGGTGAAGGAGTTGAGTGAGGCGTCTATGAAGGCGGTTCGAGAAGGTGGTTCATCTAAGGCTAATATACACGATTTCCTT
AACGAGCTGTCTACGTTGAGATCGTTAAGGCAGGCTTGA
QA-GalT (SoC3Gal) nucleotide sequence
SEQ ID NO: 12
MGSNTEATEIPKMPLKIVFLTLPIAGHMLHIVDTASTFAIHGVECTIITTPANVPFIEKSISATNTTIRQFLSIRLVDFP
HEAVGLPPGVENFSAVTCPDMRPKISKGLSIIQKPTEDLIKEISPDCIVSDMFYPWTSDFALEIGVPRVVERGCGMFPMC
CWHSIKSHLPHEKVDRDDEMIVLPTLPDHIEMRKSTLPDWVRKPTGYSYLMKMIDAAELKSYGVIVNSFSDLERDYEEYF
KNVTGLKVWTVGPISLHVGRNEELEGSDEWVKWLDGKKLDSVIYVSFGGVAKFPPHQLREIAAGLESSGHDFVWVVRASD
ENGDQAEADEWSLQKFKEKMKKTNHGLVIESWVPQLMFLEHKAIGGMLTHVGWGTMLEGITAGLPLVTWPLYAEQFYNER
LVVDVLKIGVGVGVKEFCGLDDIGKKETIGRENIEASVRLVMGDGEEAAAMRLRVKELSEASMKAVREGGSSKANIHDEL
NELSTLRSLRQA*
QA-GalT (SoC3Gal) amino acid sequence
SEQ ID NO: 13
ATGAAGTCACCACTAAAGTTGTACTTCCTGCCATACATATCACCAGGCCATATGATCCCACTTTCCGAAATGGCTCGGTT
ATTCGCCAACCAAGGGCACCACGTGACCATCATCACCACCACCTCGAACGCCACCCTCCTCCAAAAATACACCACCGCCA
CCCTGTCTCTACATCTTATTCCCCTCCCTACCAAAGAGGCCGGCCTTCCAGACGGCCTCGAAAACTTCATTTCTGTCAAC
GATCTTGAAACCGCTGGCAAACTCTACTACGCTCTTTCCCTCCTGCAACCCGTCATTGAGGAGTTTATCACGTCTAACCC
GCCCGATTGTATCGTGTCCGACATGTTCTATCCCTGGACTGCGGACCTGGCGTCCCAACTCCAGGTCCCGCGTATGGTCT
TTCATGCAGCGTGTATATTCGCTATGTGCATGAAAGAGTCAATGCGGGGCCCTGACGCCCCGCATCTGAAGGTCAGCTCT
GATTATGAGCTGTTTGAAGTCAAGGGGCTACCGGACCCGGTTTTTATGACCCGGGCCCAGCTCCCTGACTACGTGCGTAC
CCCAAACGGGTACACACAGCTCATGGAGATGTGGCGAGAAGCGGAAAAGAAAAGTTACGGTGTTATGGTTAATAATTTTT
ACGAACTTGACCCGGCTTATACCGAGCATTATAGTAAGATTATGGGCCATAAGGTCTGGAATATTGGGCCTGCGGCCCAA
ATTCTTCACCGTGGTTCTGGTGATAAAATCGAGAGGGTTCACAAAGCCGTTGTTGGTGAAAACCAATGCTTGAGTTGGCT
CGACACTAAGGAACCTAACTCGGTTTTTTACGTCTGCTTTGGGAGCGCGATTAGGTTCCCTGATGATCAGCTCTACGAAA
TTGCTAGCGCGCTAGAATCATCTGGCGCGCAGTTTATATGGGCCGTTCTTGGAAAAGACTCGGATAATTCAGACTCGAAC
TCAGACTCAGAATGGCTGCCTGCAGGGTTCGAGGAAAAAATGAAGGAAACGGGTAGAGGGATGATAATACGAGGTTGGGC
CCCACAGGTGTTGATATTGGACCACCCGTCTGTAGGCGGGTTTATGACTCACTGTGGCTGGAACTCGACAATTGAGGGGG
TTAGCGCGGGGGTGGGGATGGTGACATGGCCGTTGTATGCGGAACAATTTTACAATGAGAAGTTAATAACACAAGTGCTT
AAGATAGGGGTGGAGGCCGGGGTGGAGGAGTGGAACTTGTGGGTGGATGTTGGGAGGAAATTGGTGAAGAGAGAGAAGAT
CGAGGCGGCAATTAGGGCGGTGATGGGTGAGGCCGGGGTGGAGATGAGGAGGAAGGCGAAAGAGTTGAGTGTCAAGGCTA
AGAAGGCGGTGCAGGATGGTGGGTCGTCTCACCGTAATTTAATGGCTTTGATCGAAGATCTGCAGAGGATTAGAGATGAT
AAAATGAGTAAGGTTGCTAATTAG
SoQA-R XyIT (SoC3Xyl) nucleotide sequence
SEQ ID NO: 14
MKSPLKLYFLPYISPGHMIPLSEMARLFANQGHHVTIITTTSNATLLQKYTTATLSLHLIPLPTKEAGLPDGLENFISVN
DLETAGKLYYALSLLQPVIEEFITSNPPDCIVSDMFYPWTADLASQLQVPRMVFHAACIFAMCMKESMRGPDAPHLKVSS
DYELFEVKGLPDPVFMTRAQLPDYVRTPNGYTQLMEMWREAEKKSYGVMVNNFYELDPAYTEHYSKIMGHKVWNIGPAAQ
ILHRGSGDKIERVHKAVVGENQCLSWLDTKEPNSVFYVCFGSAIRFPDDQLYEIASALESSGAQFIWAVLGKDSDNSDSN
SDSEWLPAGFEEKMKETGRGMIIRGWAPQVLILDHPSVGGFMTHCGWNSTIEGVSAGVGMVTWPLYAEQFYNEKLITQVL
KIGVEAGVEEWNLWVDVGRKLVKREKIEAAIRAVMGEAGVEMRRKAKELSVKAKKAVQDGGSSHRNLMALIEDLQRIRDD
KMSKVAN*
SoQA-R XyIT (SoC3Xyl) amino acid sequence
SEQ ID NO: 15
ATGTCGGATCAAAATGATAAAAAGGTCGAAATAATAGTATTTCCATACCATGGCCAAGGTCACATGAACACCATGCTACA
ATTCGCCAAACGAATTGCGTGGAAAAACGCCAAAGTTACAATCGCTACGACATTGTCCACCACTAATAAAATGAAGTCCA
AGGTCGAGAATGCCTGGGGCACTTCTATAACCTTGGACTCCATTTACGATGACTCTGACGAGTCGCAGATAAAATTCATG
GACCGTATGGCCAGGTTTGAGGCTGCTGCAGCCTCGAGCCTGTCCAAACTCCTGGTCCAGAAAAAAGAAGAAGCTGACAA
CAAAGTCTTGTTGGTTTACGACGGGAATTTGCCGTGGGCGCTGGATATCGCCCACGAGCATGGCGTGCGTGGGGCCGCGT
TTTTTCCACAGTCGTGTGCGACGGTCGCCACGTACTACTCGTTGTATCAAGAGACGCAGGGGAAGGAGCTAGAGACGGAG
TTGCCGGCGGTGTTTCCGCCGTTGGAGTTGATACAACGGAATGTACCGAATGTGTTTGGATTGAAGTTTCCGGAGGCGGT
TGTGGCTAAGAATGGGAAGGAGTATAGTCCTTTTGTGTTGTTTGTGTTGAGGCAGTGTATTAACCTTGAGAAGGCTGATT
TGCTGCTTTTCAATCAGTTTGATAAGTTGGTTGAACCTGGGGAGGTTCTGCAATGGATGTCGAAGATATTCAACGTAAAG
ACAATCGGACCGACACTTCCATCTTCATACATCGACAAACGAATCAAAGACGACGTGGACTACGGTTTCCACGCATTCAA
CCTCGACAACAACTCCTGCATCAATTGGCTTAACTCCAAACCCGCTCGCTCTGTCATCTACATAGCATTTGGGAGCAGCG
TCCACTACAGCGTTGAGCAAATGACCGAAATAGCCGAGGCCTTAAAGAGCCAACCGAACAATTTCCTTTGGGCAGTCCGA
GAAACCGAACAAAAGAAACTCCCTGAAGACTTCGTCCAACAAACCTCGGAAAAAGGGTTAATGCTCTCATGGTGCCCTCA
ATTAGATGTTTTGGTGCATGAATCAATCAGTTGTTTTGTGACACATTGTGGTTGGAACTCGATTACAGAGGCACTTAGCT
TCGGGGTACCAATGCTGTCAGTGCCACAGTTTTTGGACCAGCCTGTTGATGCTCACTTTGTGGAACAGGTTTGGGGTGCT
GGAATTACGGTCAAGAGGAGCGAAGACGGTTTGGTTACTCGAGACGAAATTGTTCGGTGCTTGGAGGTGTTAAATAATGG
CGAAAAGGCGGAGGAAATTAAGGCGAATGTGGCGAGGTGGAAGGTTTTGGCTAAGGAAGCTTTGGATGAAGGTGGTAGTT
CTGATAAGCACATTGACGAAATTATTGAGTGGGTTTCATCTTTCTAA
QATriFuT (SoC28F) nucleotide sequence
SEQ ID NO: 16
MSDQNDKKVEIIVFPYHGQGHMNTMLQFAKRIAWKNAKVTIATTLSTTNKMKSKVENAWGTSITLDSIYDDSDESQIKFM
DRMARFEAAAASSLSKLLVQKKEEADNKVLLVYDGNLPWALDIAHEHGVRGAAFFPQSCATVATYYSLYQETQGKELETE
LPAVFPPLELIQRNVPNVFGLKFPEAVVAKNGKEYSPFVLFVLRQCINLEKADLLLFNQFDKLVEPGEVLQWMSKIFNVK
TIGPTLPSSYIDKRIKDDVDYGFHAFNLDNNSCINWLNSKPARSVIYIAFGSSVHYSVEQMTEIAEALKSQPNNFLWAVR
ETEQKKLPEDFVQQTSEKGLMLSWCPQLDVLVHESISCFVTHCGWNSITEALSFGVPMLSVPQFLDQPVDAHFVEQVWGA
GITVKRSEDGLVTRDEIVRCLEVLNNGEKAEEIKANVARWKVLAKEALDEGGSSDKHIDEIIEWVSSF*
QATriFuT (SoC28F) amino acid sequence
SEQ ID NO: 17
ATGTCTGCCAAAATGTTGCACGTAGTTATGTACCCATGGTTCGCATACGGTCACATGATCCCATTTTTACATTTATCGAA
CAAATTAGCCGAAACCGGTCACAAAGTCACGTACATACTCCCCCCAAAAGCGCTAACCCGCTTACAAAACCTCAACCTAA
ATCCGACCCAAATCACGTTCCGGACCATCACGGTCCCCCGAGTTGATGGGTTACCCGCTGGTGCCGAGAACGTGACCGAT
ATTCCGGATATTACTCTGCATACTCATTTGGCCACGGCGCTGGATCGAACCCGACCCGAATTTGAGACGATTGTCGAGTT
GATTAAGCCGGATGTGATAATGTATGACGTGGCGTATTGGGTGCCAGAGGTGGCGGTGAAGTATGGGGCGAAGAGTGTTG
CGTATAGTGTGGTGTCGGCGGCAAGTGTGTCGCTGAGTAAGACGGTGGTTGATCGGATGACGCCGTTGGAGAAACCGATG
ACGGAGGAGGAGAGGAAGAAGAAGTTTGCTCAGTATCCTCACTTAATTCAGCTTTATGGTCCTTTTGGTGAAGGTATCAC
CATGTACGACCGTCTAACAGGCATGCTTAGCAAGTGTGACGCTATAGCTTGTAGGACCTGCCGTGAGATTGAAGGCAAGT
ATTGCCAATATTTATCCACTCAATATGAAAAGAAAGTCACCCTTACCGGCCCGGTTCTTCCCGAGCCGGAAGTCGGGGCC
ACACTGGAGGCCCCTTGGTCCGAGTGGCTTAGTCGGTTCAAGCTTGGTTCGGTTTTATTTTGTGCCTTTGGGAGCCAATT
TTACTTGGACAAGGACCAGTTCCAGGAAATCATCCTCGGGCTTGAAATGACAAATTTACCCTTTCTGATGGCTGTTCAGC
CCCCTAAGGGTTGCGCCACTATCGAGGAGGCGTACCCTGAGGGGTTTGCTGAGCGGGTCAAGGACCGAGGAGTCGTGACA
AGCCAGTGGGTGCAACAGCTGGTTATACTGGCCCACCCAGCGGTTGGGTGCTTTGTGAACCATTGCGCGTTTGGGACAAT
GTGGGAGGCCTTATTGAGCGAAAAGCAGTTGGTGATGATCCCTCAACTAGGTGACCAAATACTGAACACCAAAATGTTGG
CCGATGAATTGAAAGTCGGGGTTGAAGTCGAGAGAGGAATCGGTGGGTGGGTGTCTAAGGAGAATTTGTGTAAGGCGATC
AAGTCCGTCATGGACGAGGATAGTGAAATTGGCAAGGACGTGAAACAAAGTCATGAAAAATGGAGGGCGACTTTGTCGAG
CAAAGATTTAATGTCGACTTATATTGATAGTTTCATCAAAGATTTACAAGCACTCGTCGAGTGA
QA-TriFR (SoC28Rha) nucleotide sequence
SEQ ID NO: 18
MSAKMLHVVMYPWFAYGHMIPFLHLSNKLAETGHKVTYILPPKALTRLQNLNLNPTQITFRTITVPRVDGLPAGAENVTD
IPDITLHTHLATALDRTRPEFETIVELIKPDVIMYDVAYWVPEVAVKYGAKSVAYSVVSAASVSLSKTVVDRMTPLEKPM
TEEERKKKFAQYPHLIQLYGPFGEGITMYDRLTGMLSKCDAIACRTCREIEGKYCQYLSTQYEKKVTLTGPVLPEPEVGA
TLEAPWSEWLSRFKLGSVLFCAFGSQFYLDKDQFQEIILGLEMTNLPFLMAVQPPKGCATIEEAYPEGFAERVKDRGVVT
SQWVQQLVILAHPAVGCFVNHCAFGTMWEALLSEKQLVMIPQLGDQILNTKMLADELKVGVEVERGIGGWVSKENLCKAI
KSVMDEDSEIGKDVKQSHEKWRATLSSKDLMSTYIDSFIKDLQALVE*
QA-TriFR (SoC28Rha) amino acid sequence
SEQ ID NO: 19
ATGGGTACTAAAGAGTTACACATAGTAATGTACCCATGGCTAGCATTTGGTCATTTCATACCATACCTTCATCTCTCTAA
CAAACTCGCTCAAAAAGGCCATAAAATCACTTTCTTACTTCCTCATAGAGCCAAACTTCAACTTGACTCCCAAAATTTAT
ATCCCTCACTTATTACCCTCGTACCAATTACCGTCCCACAGGTCGACACCCTTCCTCTCGGGGCCGAATCGACTGCTGAT
ATCCCCCTTAGTCAGCACGGTGACCTCTCCATCGCCATGGACCGTACTCGACCCGAGATTGAGTCTATCTTGTCTAAACT
TGACCCAAAACCGGACCTGATTTTCTTCGATATGGCGCAGTGGGTGCCTGTCATAGCGTCTAAGCTTGGGATCAAGTCTG
TTTCGTATAATATCGTTTGCGCCATTTCGTTGGACCTTGTTCGAGATTGGTATAAGAAGGATGATGGAAGTAATGTGCCT
AGTTGGACATTGAAGCATGACAAGTCATCCCATTTCGGGGAGAATATTAGTATTCTCGAGCGAGCGCTGATTGCGCTCGG
GACGCCTGATGCCATAGGCATCAGGTCGTGTCGGGAGATAGAGGGGGAGTACTGTGACAGCATAGCGGAACGATTTAAGA
AACCGGTCTTACTAAGCGGGACGACCTTACCTGAACCATCCGACGACCCACTTGACCCAAAATGGGTCAAGTGGCTCGGA
AAGTTCGAGGAAGGTTCGGTTATTTTTTGCTGCCTAGGGAGTCAGCACGTGTTAGACAAGCCCCAGCTCCAGGAGCTGGC
GCTGGGGCTTGAAATGACGGGGTTGCCATTCTTCCTAGCGATTAAACCACCGCTAGGATACGCAACCCTAGACGAGGTAC
TACCCGAGGGGTTTTCAGAACGGGTTCGAGATCGAGGGGTGGCTCATGGGGGATGGGTACAACAGCCTCAGATGCTGGCA
CACCCTTCTGTAGGGTGCTTTTTGTGTCACTGTGGGTCGTCGTCGATGTGGGAGGCATTAGTGAGTGATACGCAGCTCGT
ATTGTTTCCTCAAATACCAGATCAAGCTCTAAACGCGGTTTTAATGGCGGATAAACTTAAGGTCGGGGTGAAGGTCGAGA
GAGAGGACGACGGAGGGGTGTCGAAAGAGGTTTGGAGTAGAGCAATAAAGAGTGTGATGGATAAGGAGAGTGAAATTGCT
GCGGAAGTGAAGAAGAATCATACTAAGTGGAGAGATATGTTGATTAATGAAGAATTTGTGAATGGGTACATTGACAGTTT
CATTAAGGATCTACAAGATCTTGTTGAGAAGTAG
SoQA-TriFRXyIT (SoC28Xyl1) nucleotide sequence
SEQ ID NO: 20
MGTKELHIVMYPWLAFGHFIPYLHLSNKLAQKGHKITFLLPHRAKLQLDSQNLYPSLITLVPITVPQVDTLPLGAESTAD
IPLSQHGDLSIAMDRTRPEIESILSKLDPKPDLIFFDMAQWVPVIASKLGIKSVSYNIVCAISLDLVRDWYKKDDGSNVP
SWTLKHDKSSHFGENISILERALIALGTPDAIGIRSCREIEGEYCDSIAERFKKPVLLSGTTLPEPSDDPLDPKWVKWLG
KFEEGSVIFCCLGSQHVLDKPQLQELALGLEMTGLPFFLAIKPPLGYATLDEVLPEGFSERVRDRGVAHGGWVQQPQMLA
HPSVGCFLCHCGSSSMWEALVSDTQLVLFPQIPDQALNAVLMADKLKVGVKVEREDDGGVSKEVWSRAIKSVMDKESEIA
AEVKKNHTKWRDMLINEEFVNGYIDSFIKDLQDLVEK*
SoQA-TriFRXyIT (SoC28Xyl1) amino acid sequence
SEQ ID NO: 21
ATGGAGGAATCAAAGGAGGAAGTACATGTAGCATTCTTCCCATTCATGACACCAGGTCACTCAATCCCAATGCTAGACTT
GGTACGTTTGTTCATTGCTCGTGGTGTCAAAACTACTGTCTTCACTACTCCTCTTAATGCTCCTAATATTTCCAAATACC
TCAACATTATCCAAGATTCCTCATCAAACAAAAACACCATTTATGTAACTCCTTTTCCTTCTAAAGAAGCCGGTTTACCG
GAAGGTGTGGAAAGCCAGGATAGTACCACTTCCCCCGAAATGACCCTCAAGTTCTTTGTTGCTATGGAATTACTTCAAGA
CCCCCTTGATGTTTTTTTAAAAGAAACCAAACCTCATTGTCTTGTTGCTGATAATTTCTTCCCTTACGCCACCGACATCG
CTTCTAAGTATGGCATTCCTAGGTTTGTTTTTCAGTTCACTGGCTTCTTTCCTATGTCTGTCATGATGGCCTTAAATCGT
TTCCACCCTCAAAACTCTGTATCATCTGATGACGACCCCTTTCTTGTTCCCAGTTTACCCCATGACATCAAATTGACTAA
GTCACAATTGCAACGAGAGTACGAGGGTAGTGATGGTATTGACACCGCTCTTTCTAGGCTCTGTAATGGCGCCGGTAGAG
CTTTGTTTACTAGTTATGGTGTCATTTTTAACAGCTTCTACCAACTCGAACCTGATTATGTTGATTATTATACCAACACC
ATGGGGAAACGATCCAGGGTTTGGCATGTGGGCCCAGTGTCGTTATGCAACCGTCGACACGTGGAGGGTAAATCTGGTAG
GGGGAGAAGTGCTTCAATTAGTGAGCATTTGTGCTTAGAGTGGCTCAATGCCAAAGAACCAAATTCAGTGATATATGTAT
GTTTTGGTAGTCTCACATGTTTCTCCAATGAGCAACTCAAAGAAATCGCAACCGCCTTAGAAAGGTGTGAAGAGTATTTT
ATATGGGTGTTGAAGGGTGGCAAAGATAATGAGCAAGAGTGGTTGCCACAAGGGTTTGAAGAGAGGGTTGAAGGGAAAGG
ACTAATCATACGGGGGTGGGCCCCACAAGTGTTGATTTTAGACCATGAAGCCATAGGCGGGTTTGTGACACACTGTGGTT
GGAACTCGACACTAGAAAGTATATCAGCGGGGGTGCCCATGGTGACATGGCCCATATATGCAGAGCAATTTTATAATGAG
AAATTGGTGACGGATGTACTGAAGGTGGGGGTTAAAGTAGGGTCAATGAAGTGGAGTGAGACGACGGGGGCGACTCATTT
AAAGCATGAGGAAATAGAAAAAGCATTGAAGCAAATAATGGTGGGAGAAGAGGTGTTAGAGATGAGAAAAAGAGCAAGTA
AGTTGAAAGAGATGGCTTATAATGCTGTTGAAGAAGGAGGCTCTTCTTATTCTCACCTCACTTCCTTAATCGACGACCTT
ATGGCTTCCAAAGCTGTGCTACAAAAATTTTGA
SoQA-TriFRXXyIT (SoC28Xyl2) nucleotide sequence
SEQ ID NO: 22
MEESKEEVHVAFFPFMTPGHSIPMLDLVRLFIARGVKTTVFTTPLNAPNISKYLNIIQDSSSNKNTIYVTPFPSKEAGLP
EGVESQDSTTSPEMTLKFFVAMELLQDPLDVFLKETKPHCLVADNFFPYATDIASKYGIPRFVFQFTGFFPMSVMMALNR
FHPQNSVSSDDDPFLVPSLPHDIKLTKSQLQREYEGSDGIDTALSRLCNGAGRALFTSYGVIENSFYQLEPDYVDYYTNT
MGKRSRVWHVGPVSLCNRRHVEGKSGRGRSASISEHLCLEWLNAKEPNSVIYVCFGSLTCFSNEQLKEIATALERCEEYF
IWVLKGGKDNEQEWLPQGFEERVEGKGLIIRGWAPQVLILDHEAIGGFVTHCGWNSTLESISAGVPMVTWPIYAEQFYNE
KLVTDVLKVGVKVGSMKWSETTGATHLKHEEIEKALKQIMVGEEVLEMRKRASKLKEMAYNAVEEGGSSYSHLTSLIDDL
MASKAVLQKF*
SoQA-TriFRXXyIT (SoC28Xyl2) amino acid sequence

The full-length HMGR sequence is provided below. The 5′ region (underlined) can be removed to generate a truncated feedback-insensitive form (tHMGR). The sequence for tHMGR is also given separately below.

ATGGCTGTGGAGGTTCACCGCCGGGCTCCCGCGCCCCATGGCCGGGGCACCGGGGAGAAGGGCCGCGTGCAGGCCGGGGA
CGCGCTGCCGCTGCCGATCCGCCACACCAACCTCATCTTCTCGGCGCTCTTCGCCGCCTCCCTCGCATACCTCATGCGCC
GCTGGAGGGAGAAGATCCGCAACTCCACGCCGCTCCACGTCGTGGGGCTCACCGAGATCTTCGCCATCTGCGGCCTCGTC
GCCTCCCTCATCTACCTCCTCAGCTTCTTCGGCATCGCCTTCGTGCAGTCCGTCGTATCCAACAGCGACGACGAGGACGA
GGACTTCCTCATCGCGGCTGCAGCATCCCAGGCCCCCCCGCCGCCCTCCTCCAAGCCCGCGCCGCAGCAGTGCGCCCTGC
TGCAGAGCGCCGGAGTCGCGCCCGAGAAAATGCCCGAGGAGGACGAGGAAATCGTCGCCGGGGTCGTCGCAGGGAAGATC
CCCTCCTACGTGCTCGAGACCAGGCTAGGCGACTGCCGCAGGGCAGCCGGGATCCGCCGCGAGGCGCTGCGCCGGATCAC
CGGCAGGGAGATCGACGGCCTTCCCCTCGACGGCTTCGACTACGACTCGATTCTCGGACAGTGCTGCGAGATGCCCGTCG
GGTACGTGCAGCTGCCGGTCGGCGTCGCGGGGCCGCTCGTCCTCGACGGCCGCCGCATATACGTCCCGATGGCCACCACG
GAGGGCTGCCTAATCGCCAGCACCAACCGCGGATGCAAGGCCATTGCCGAGTCCGGAGGCGCATCCAGCGTCGTGTACCG
CGACGGGATGACCCGCGCCCCCGTAGCCCGCTTCCCCTCCGCACGACGCGCCGCAGAGCTCAAGGGCTTCCTGGAGAATC
CGGCCAACTACGACACCCTGTCCGTGGTCTTTAACAGATCAAGCAGATTTGCAAGGCTGCAGGGGGTCAAGTGCGCCATG
GCTGGGAGGAACTTGTACATGAGGTTCACCTGCAGCACCGGGGATGCCATGGGGATGAACATGGTCTCCAAGGGCGTCCA
AAATGTGCTCGACTATCTGCAGGAGGACTTCCCTGACATGGACGTTGTCAGCATCTCAGGCAACTTTTGTTCCGACAAGA
AATCAGCTGCTGTAAACTGGATTGAAGGCCGTGGAAAGTCCGTGGTTTGTGAGGCAGTAATCAGAGAGGAAGTTGTCCAC
AAGGTTCTCAAGACCAACGTTCAGTCACTCGTGGAGTTGAATGTGATCAAGAACCTTGCTGGCTCAGCAGTTGCTGGTGC
TCTTGGGGGTTTCAACGCCCACGCAAGCAACATCGTAACGGCTATCTTCATTGCCACTGGTCAGGATCCTGCACAGAATG
TGGAGAGCTCACAGTGTATCACTATGTTGGAAGCTGTAAATGATGGCAGAGACCTTCACATCTCCGTTACAATGCCATCT
ATCGAGGTGGGCACAGTTGGTGGAGGCACGCAGCTGGCCTCACAGTCGGCCTGCTTGGACCTACTGGGCGTCAAAGGCGC
CAACAGGGAATCTCCGGGGTCGAACGCTAGGCTGCTGGCCACGGTGGTGGCTGGTGCCGTCCTAGCTGGGGAGCTGTCCC
TCATCTCCGCCCAAGCTGCCGGCCATCTGGTCCAGAGCCACATGAAATACAACAGATCCAGCAAGGACATGTCCAAGATC
GCCTGCTGA
SEQ ID NO: 23-AsHMGR (Avena strigosa HMG-CoA reductase) coding sequence (1689 bp):
MAVEVHRRAPAPHGRGTGEKGRVQAGDALPLPIRHTNLIFSALFAASLAYLMRRWREKIRNSTPLHVVGLTEIFAICGLV
ASLIYLLSFFGIAFVQSVVSNSDDEDEDFLIAAAASQAPPPPSSKPAPQQCALLQSAGVAPEKMPEEDEEIVAGVVAGKI
PSYVLETRLGDCRRAAGIRREALRRITGREIDGLPLDGFDYDSILGQCCEMPVGYVQLPVGVAGPLVLDGRRIYVPMATT
EGCLIASTNRGCKAIAESGGASSVVYRDGMTRAPVARFPSARRAAELKGFLENPANYDTLSVVFNRSSRFARLQGVKCAM
AGRNLYMRFTCSTGDAMGMNMVSKGVQNVLDYLQEDFPDMDVVSISGNFCSDKKSAAVNWIEGRGKSVVCEAVIREEVVH
KVLKTNVQSLVELNVIKNLAGSAVAGALGGFNAHASNIVTAIFIATGQDPAQNVESSQCITMLEAVNDGRDLHISVTMPS
IEVGTVGGGTQLASQSACLDLLGVKGANRESPGSNARLLATVVAGAVLAGELSLISAQAAGHLVQSHMKYNRSSKDMSKI
AC*
SEQ ID NO: 24-AsHMGR (Avena strigosa HMG-CoA reductase) translated nucleotide
sequence (562 aa)
ATGGCGCCCGAGAAAATGCCCGAGGAGGACGAGGAAATCGTCGCCGGGGTCGTCGCAGGGAAGATCCCCTCCTACGTGCT
CGAGACCAGGCTAGGCGACTGCCGCAGGGCAGCCGGGATCCGCCGCGAGGCGCTGCGCCGGATCACCGGCAGGGAGATCG
ACGGCCTTCCCCTCGACGGCTTCGACTACGACTCGATTCTCGGACAGTGCTGCGAGATGCCCGTCGGGTACGTGCAGCTG
CCGGTCGGCGTCGCGGGGCCGCTCGTCCTCGACGGCCGCCGCATATACGTCCCGATGGCCACCACGGAGGGCTGCCTAAT
CGCCAGCACCAACCGCGGATGCAAGGCCATTGCCGAGTCCGGAGGCGCATCCAGCGTCGTGTACCGCGACGGGATGACCC
GCGCCCCCGTAGCCCGCTTCCCCTCCGCACGACGCGCCGCAGAGCTCAAGGGCTTCCTGGAGAATCCGGCCAACTACGAC
ACCCTGTCCGTGGTCTTTAACAGATCAAGCAGATTTGCAAGGCTGCAGGGGGTCAAGTGCGCCATGGCTGGGAGGAACTT
GTACATGAGGTTCACCTGCAGCACCGGGGATGCCATGGGGATGAACATGGTCTCCAAGGGCGTCCAAAATGTGCTCGACT
ATCTGCAGGAGGACTTCCCTGACATGGACGTTGTCAGCATCTCAGGCAACTTTTGTTCCGACAAGAAATCAGCTGCTGTA
AACTGGATTGAAGGCCGTGGAAAGTCCGTGGTTTGTGAGGCAGTAATCAGAGAGGAAGTTGTCCACAAGGTTCTCAAGAC
CAACGTTCAGTCACTCGTGGAGTTGAATGTGATCAAGAACCTTGCTGGCTCAGCAGTTGCTGGTGCTCTTGGGGGTTTCA
ACGCCCACGCAAGCAACATCGTAACGGCTATCTTCATTGCCACTGGTCAGGATCCTGCACAGAATGTGGAGAGCTCACAG
TGTATCACTATGTTGGAAGCTGTAAATGATGGCAGAGACCTTCACATCTCCGTTACAATGCCATCTATCGAGGTGGGCAC
AGTTGGTGGAGGCACGCAGCTGGCCTCACAGTCGGCCTGCTTGGACCTACTGGGCGTCAAAGGCGCCAACAGGGAATCTC
CGGGGTCGAACGCTAGGCTGCTGGCCACGGTGGTGGCTGGTGCCGTCCTAGCTGGGGAGCTGTCCCTCATCTCCGCCCAA
GCTGCCGGCCATCTGGTCCAGAGCCACATGAAATACAACAGATCCAGCAAGGACATGTCCAAGATCGCCTGCTGA
SEQ ID NO: 25-AstHMGR (Avena strigosa truncated HMG-CoA reductase) coding
sequence (1275 bp):
MAPEKMPEEDEEIVAGVVAGKIPSYVLETRLGDCRRAAGIRREALRRITGREIDGLPLDGFDYDSILGQCCEMPVGYVQL
PVGVAGPLVLDGRRIYVPMATTEGCLIASTNRGCKAIAESGGASSVVYRDGMTRAPVARFPSARRAAELKGFLENPANYD
TLSVVFNRSSRFARLQGVKCAMAGRNLYMRFTCSTGDAMGMNMVSKGVQNVLDYLQEDFPDMDVVSISGNFCSDKKSAAV
NWIEGRGKSVVCEAVIREEVVHKVLKTNVQSLVELNVIKNLAGSAVAGALGGFNAHASNIVTAIFIATGQDPAQNVESSQ
CITMLEAVNDGRDLHISVTMPSIEVGTVGGGTQLASQSACLDLLGVKGANRESPGSNARLLATVVAGAVLAGELSLISAQ
AAGHLVQSHMKYNRSSKDMSKIAC*
SEQ ID NO: 26-AstHMGR (Avena strigosa truncated HMG-CoA reductase) translated
nucleotide sequence (424 aa):
ATGGGGGCGCTGTCGCGGCCGGAGGAGGTGGTGGCGCTGGTCAAGCTGAGGGTGGCGGCGGGGCAGATCAAGCGCCAGAT
CCCGGCCGAGGAACACTGGGCCTTCGCCTACGACATGCTCCAGAAGGTCTCCCGCAGCTTCGCGCTCGTCATCCAGCAGC
TCGGACCCGAACTCCGCAATGCCGTGTGCATCTTCTACCTCGTGCTCCGGGCCCTGGACACCGTCGAGGACGACACCAGC
ATCCCCAACGACGTGAAGCTGCCCATCCTTCGGGATTTCTACCGCCATGTCTACAACCCCGACTGGCGTTATTCATGTGG
AACAAACCACTACAAGGTGCTGATGGATAAGTTCAGACTCGTCTCCACGGCTTTCCTGGAGCTAGGCGAAGGATATCAAA
AGGCAATTGAAGAAATCACTAGGCGAATGGGAGCAGGAATGGCAAAATTTATATGCCAGGAGGTTGAAACGATTGATGAC
TATAATGAGTACTGCCACTATGTAGCAGGGCTAGTAGGCTATGGACTTTCCAGGCTCTTTCATGCTGCTGGGACAGAAGA
TCTGGCTTCAGATCAACTTTCGAATTCAATGGGTTTGTTTCTTCAGAAAACCAATATAATAAGGGATTATTTGGAGGATA
TAAATGAGATACCAAAGTGCCGTATGTTTTGGCCTCGAGAAATATGGAGTAAATATGCAGATAAACTTGAGGACCTCAAG
TATGAGGAAAATTCAGAAAAAGCAGTGCAATGCTTGAATGATATGGTGACTAATGCTTTGGTCCACGCCGAAGACTGTCT
TCAATACATGTCTGCGTTGAAGGATAATACTAATTTTCGGTTTTGTGCAATACCTCAGATAATGGCAATTGGGACATGTG
CTATTTGCTACAATAATGTGAAAGTCTTTAGAGGAGTTGTTAAGATGAGGCGTGGGCTCACTGCACGAATAATTGATGAG
ACAAAATCAATGTCAGATGTCTATTCTGCTTTCTATGAGTTCTCTTCATTGCTAGAGTCAAAGATTGACGATAACGACCC
AAGTTCTGCACTAACACGGAAGCGTGTAGAGGCAATAAAGAGGACTTGCAAGTCATCCGGTTTACTAAAGAGAAGGGGAT
ACGACCTGGAAAAGTCAAAGTATAGGCATATGTTGATCATGCTTGCACTTCTGTTGGTGGCTATTATCTTCGGTGTACTG
TACGCCAAGTGA
SEQ ID NO: 27-AsSQS (Avena strigosa squalene synthase) coding sequence (1212 bp):
MGALSRPEEVVALVKLRVAAGQIKRQIPAEEHWAFAYDMLQKVSRSFALVIQQLGPELRNAVCIFYLVLRALDTVEDDTS
IPNDVKLPILRDFYRHVYNPDWRYSCGTNHYKVLMDKFRLVSTAFLELGEGYQKAIEEITRRMGAGMAKFICQEVETIDD
YNEYCHYVAGLVGYGLSRLFHAAGTEDLASDQLSNSMGLFLQKTNIIRDYLEDINEIPKCRMFWPREIWSKYADKLEDLK
YEENSEKAVQCLNDMVTNALVHAEDCLQYMSALKDNTNFRFCAIPQIMAIGTCAICYNNVKVFRGVVKMRRGLTARIIDE
TKSMSDVYSAFYEFSSLLESKIDDNDPSSALTRKRVEAIKRTCKSSGLLKRRGYDLEKSKYRHMLIMLALLLVAIIFGVL
YAK*
SEQ ID NO: 28-AsSQS (Avena strigosa squalene synthase) translated nucleotide
sequence (403 aa):
ATGAAAAACATGATGAATTATAAATTAAAACTCTGTTCTGTCTCAAAAAACTCAAAAGGAGTCTCTCTCTCACCTACACC
ACACCTAACCAAACCCCCTACGATTCACACAGAGAGAGATCTTCTTCTTCCTTCTTCTTCCTTCTTCTTTCTTCTTCTTT
CTTCTTCTAGCTACAACATCTACAACGCCATGTCCTCTTCTTCTTCTTCGTCAACCTCCATGATCGATCTCATGGCAGCA
ATCATCAAAGGAGAGCCTGTAATTGTCTCCGACCCAGCTAATGCCTCCGCTTACGAGTCCGTAGCTGCTGAATTATCCTC
TATGCTTATAGAGAATCGTCAATTCGCCATGATTGTTACCACTTCCATTGCTGTTCTTATTGGTTGCATCGTTATGCTCG
TTTGGAGGAGATCCGGTTCTGGGAATTCAAAACGTGTCGAGCCTCTTAAGCCTTTGGTTATTAAGCCTCGTGAGGAAGAG
ATTGATGATGGGCGTAAGAAAGTTACCATCTTTTTCGGTACACAAACTGGTACTGCTGAAGGTTTTGCAAAGGCTTTAGG
AGAAGAAGCTAAAGCAAGATATGAAAAGACCAGATTCAAAATCGTTGATTTGGATGATTACGCGGCTGATGATGATGAGT
ATGAGGAGAAATTGAAGAAAGAGGATGTGGCTTTCTTCTTCTTAGCCACATATGGAGATGGTGAGCCTACCGACAATGCA
GCGAGATTCTACAAATGGTTCACCGAGGGGAATGACAGAGGAGAATGGCTTAAGAACTTGAAGTATGGAGTGTTTGGATT
AGGAAACAGACAATATGAGCATTTTAATAAGGTTGCCAAAGTTGTAGATGACATTCTTGTCGAACAAGGTGCACAGCGTC
TTGTACAAGTTGGTCTTGGAGATGATGACCAGTGTATTGAAGATGACTTTACCGCTTGGCGAGAAGCATTGTGGCCCGAG
CTTGATACAATACTGAGGGAAGAAGGGGATACAGCTGTTGCCACACCATACACTGCAGCTGTGTTAGAATACAGAGTTTC
TATTCACGACTCTGAAGATGCCAAATTCAATGATATAAACATGGCAAATGGGAATGGTTACACTGTGTTTGATGCTCAAC
ATCCTTACAAAGCAAATGTCGCTGTTAAAAGGGAGCTTCATACTCCCGAGTCTGATCGTTCTTGTATCCATTTGGAATTT
GACATTGCTGGAAGTGGACTTACGTATGAAACTGGAGATCATGTTGGTGTACTTTGTGATAACTTAAGTGAAACTGTAGA
TGAAGCTCTTAGATTGCTGGATATGTCACCTGATACTTATTTCTCACTTCACGCTGAAAAAGAAGACGGCACACCAATCA
GCAGCTCACTGCCTCCTCCCTTCCCACCTTGCAACTTGAGAACAGCGCTTACACGATATGCATGTCTTTTGAGTTCTCCA
AAGAAGTCTGCTTTAGTTGCGTTGGCTGCTCATGCATCTGATCCTACCGAAGCAGAACGATTAAAACACCTTGCTTCACC
TGCTGGAAAGGATGAATATTCAAAGTGGGTAGTAGAGAGTCAAAGAAGTCTACTTGAGGTGATGGCCGAGTTTCCTTCAG
CCAAGCCACCACTTGGTGTCTTCTTCGCTGGAGTTGCTCCAAGGTTGCAGCCTAGGTTCTATTCGATATCATCATCGCCC
AAGATTGCTGAAACTAGAATTCACGTCACATGTGCACTGGTTTATGAGAAAATGCCAACTGGCAGGATTCATAAGGGAGT
GTGTTCCACTTGGATGAAGAATGCTGTGCCTTACGAGAAGAGTGAAAACTGTTCCTCGGCGCCGATATTTGTTAGGCAAT
CCAACTTCAAGCTTCCTTCTGATTCTAAGGTACCGATCATCATGATCGGTCCAGGGACTGGATTAGCTCCATTCAGAGGA
TTCCTTCAGGAAAGACTAGCGTTGGTAGAATCTGGTGTTGAACTTGGGCCATCAGTTTTGTTCTTTGGATGCAGAAACCG
TAGAATGGATTTCATCTACGAGGAAGAGCTCCAGCGATTTGTTGAGAGTGGTGCTCTCGCAGAGCTAAGTGTCGCCTTCT
CTCGTGAAGGACCCACCAAAGAATACGTACAGCACAAGATGATGGACAAGGCTTCTGATATCTGGAATATGATCTCTCAA
GGAGCTTATTTATATGTTTGTGGTGACGCCAAAGGCATGGCAAGAGATGTTCACAGATCTCTCCACACAATAGCTCAAGA
ACAGGGGTCAATGGATTCAACTAAAGCAGAGGGCTTCGTGAAGAATCTGCAAACGAGTGGAAGATATCTTAGAGATGTAT
GGTAA
SEQ ID NO: 29-AtATR2 (Arabidopsis thaliana cytochrome P450 reductase 2)
coding sequence (2325 bp):
MKNMMNYKLKLCSVSKNSKGVSLSPTPHLTKPPTIHTERDLLLPSSSFFFLLLSSSSYNIYNAMSSSSSSSTSMIDLMAA
IIKGEPVIVSDPANASAYESVAAELSSMLIENRQFAMIVTTSIAVLIGCIVMLVWRRSGSGNSKRVEPLKPLVIKPREEE
IDDGRKKVTIFFGTQTGTAEGFAKALGEEAKARYEKTRFKIVDLDDYAADDDEYEEKLKKEDVAFFFLATYGDGEPTDNA
ARFYKWFTEGNDRGEWLKNLKYGVFGLGNRQYEHFNKVAKVVDDILVEQGAQRLVQVGLGDDDQCIEDDFTAWREALWPE
LDTILREEGDTAVATPYTAAVLEYRVSIHDSEDAKFNDINMANGNGYTVFDAQHPYKANVAVKRELHTPESDRSCIHLEF
DIAGSGLTYETGDHVGVLCDNLSETVDEALRLLDMSPDTYFSLHAEKEDGTPISSSLPPPFPPCNLRTALTRYACLLSSP
KKSALVALAAHASDPTEAERLKHLASPAGKDEYSKWVVESQRSLLEVMAEFPSAKPPLGVFFAGVAPRLQPRFYSISSSP
KIAETRIHVTCALVYEKMPTGRIHKGVCSTWMKNAVPYEKSENCSSAPIFVRQSNFKLPSDSKVPIIMIGPGTGLAPFRG
FLQERLALVESGVELGPSVLFFGCRNRRMDFIYEEELQRFVESGALAELSVAFSREGPTKEYVQHKMMDKASDIWNMISQ
GAYLYVCGDAKGMARDVHRSLHTIAQEQGSMDSTKAEGFVKNLQTSGRYLRDVW*
SEQ ID NO: 30-AtATR2 (Arabidopsis thaliana cytochrome P450 reductase 2) translated
nucleotide sequence (774aa):
ATGGCTGAAGCATCCTCATTTCTTGCACAGAAAAGGTATGCGGTCGTGACAGGAGCAAACAAAGGACTAGGACTAGAAAT
ATGCGGACAGCTTGCTTCACAGGGGGTGACGGTACTGCTGACATCCAGAGATGAAAAACGAGGCTTAGAAGCCATTGAGG
AGCTTAAGAAATCGGGGATTAATTCGGAAAATCTTGAATATCATCAGCTGGATGTTACTAAGCCAGCTAGTTTCGCTTCT
CTGGCCGATTTCATCAAGGCCAAATTTGGCAAGCTTGATATCCTGGTGAACAATGCAGGGATCAGCGGTGTTATTGTAGA
TTATGCAGCTTTAATGGAAGCCATTCGCCGTCGAGGGGCAGAGATCAATTACGATGGAGTGATGAAACAGACCTACGAGC
TAGCAGAGGAATGCTTGCAAACAAATTACTATGGTGTGAAAAGAACCATTAATGCTCTCCTTCCGCTACTTCAGTTTTCC
GATTCACCAAGGATCGTCAATGTTTCCTCCGATGTTGGCCTCCTTAAGAAAATACCCGGCGAGAGAATCAGAGAAGCCTT
AGGCGACGTGGAAAAACTTACGGAAGAAAGCGTGGACGGGATTTTAGACGAGTTTCTAAGAGATTTCAAGGAAGGCAAGA
TCGCAGAGAAAGGTTGGCCTACGTTTAAGAGCGCCTATTCAATCTCAAAGGCGGCGCTCAATTCGTACACGAGGGTTTTA
GCACGGAAATACCCGTCGATCATCATCAACTGTGTCTGCCCGGGTGTCGTCAAAACCGATATCAATCTTAAAATGGGCCA
CTTGACGGTTGAAGAAGGCGCGGCCAGTCCCGTGAGGTTAGCACTCATGCCCCTTGGTTCGCCTTCCGGCCTGTTCTATA
CTCGAAACGAAGTAACTCCATTTGAATGA
SEQ ID NO: 31 SoFuSyn coding sequence
MAEASSFLAQKRYAVVTGANKGLGLEICGQLASQGVTVLLTSRDEKRGLEAIEELKKSGINSENLEYHQLDVTKPASFAS
LADFIKAKFGKLDILVNNAGISGVIVDYAALMEAIRRRGAEINYDGVMKQTYELAEECLQTNYYGVKRTINALLPLLQFS
DSPRIVNVSSDVGLLKKIPGERIREALGDVEKLTEESVDGILDEFLRDFKEGKIAEKGWPTFKSAYSISKAALNSYTRVL
ARKYPSIIINCVCPGVVKTDINLKMGHLTVEEGAASPVRLALMPLGSPSGLFYTRNEVTPFE*
SEQ ID NO: 32 SoFuSyn translated nucleotide sequence
ATGGTTCTTAGTCGATTGGATTTTCCGTCCGATTTCATTTTTGGCTCCGGCACGTCAGCTTCTCAGGTAGAAGGTGCAGC
ACTAGAGGATGGGAAGACTTCGACTGCATTTGAAGGATTCTTAACTCGCATGAGTGGAAATGATTTGAGCAAAGGAGTTG
AAGGCTACTACAAATACAAGGAAGACGTCCAGTTAATGGTGCAAACAGGACTAGATGCATACAGATTCTCCATTTCATGG
TCAAGACTAATTCCCGGTGGAAAAGGACCCGTCAACCCAAAAGGTTTACAATATTATAATAACTTTATCGACGAACTCAT
CAAAAATGGAATACAACCGCACGTTACTCTGCTGCATTTCGACATACCGGACACACTTATGACTGCTTATAATGGATTGA
AGGGTCAAGAATTTGTGGAAGATTTCACGGCATTTGCTGACGTGTGCTTCAAGGAATTTGGTGACCGAGTTTTGTATTGG
ACGACGGTCAATGAAGCAAATAATTTTGCAAGTCTAACACTCGATGAGGGCAATTTTATGCCGTCTACTGAACCGTACAT
TAGAGGTCACAATATCATTCTTGCTCATGCATCCGCGGTAAAACTATACCGAGAAAAATATAAGAAAACCCAAAATGGAT
TCATAGGCTTGAATTTATATGCAAGCTGGTATTTTCCCGAGACCGATGACGAACAAGATTCAATTGCCGCTCAAAGAGCC
ATTGATTTTACTATTGGATGGATAATGCAACCATTGATATACGGAGAATATCCAGAAACATTGAAGAAACAAGTGGGAGA
AAGACTGCCAACATTTACAAAAGAAGAGTCAACGTTCGTTAAAAATTCGTTTGACTTCATTGGAGTGAATTGCTACGTCG
GCACTGCTGTTAAGGATGACCCTGACAGCTGTAACAGTAAAAATAAAACTATTATTACTGACATGTCTGCTAAACTTTCT
CCTAAAGGTGAACTAGGAGGAGCGTATATGAAGGGATTGTTGGAATACTTCAAAAGAGATTACGGCAATCCGCCAATTTA
CATTCAAGAAAATGGTTATTGGACACCGCGTGAATTAGGAGTGAACGATGCGTCAAGGATCGAATACCATACTGCTTCTC
TTGCTAGCATGCACGATGCTATGAAGAATGGGGCAAATGTAAAGGGATATTTCCAATGGTCATTTTTGGATCTCTTGGAG
GTGTTCAAATACAGCTATGGCCTCTACCATGTCGATTTGGAAGACCCGACCCGAGAAAGACGACCCAAGGCATCCGCCAA
TTGGTACGCGGAGTTCTTGAAGGGTTGCGCTACTTCTAACGGGAATGCTAAAGTTGAAACTCCGTTGTAA
SEQ ID NO: 33 SoGH1 coding sequence
MVLSRLDFPSDFIFGSGTSASQVEGAALEDGKTSTAFEGFLTRMSGNDLSKGVEGYYKYKEDVQLMVQTGLDAYRFSISW
SRLIPGGKGPVNPKGLQYYNNFIDELIKNGIQPHVTLLHFDIPDTLMTAYNGLKGQEFVEDFTAFADVCFKEFGDRVLYW
TTVNEANNFASLTLDEGNFMPSTEPYIRGHNIILAHASAVKLYREKYKKTQNGFIGLNLYASWYFPETDDEQDSIAAQRA
IDFTIGWIMQPLIYGEYPETLKKQVGERLPTFTKEESTFVKNSFDFIGVNCYVGTAVKDDPDSCNSKNKTIITDMSAKLS
PKGELGGAYMKGLLEYFKRDYGNPPIYIQENGYWTPRELGVNDASRIEYHTASLASMHDAMKNGA
SEQ ID NO: 34 SoGH1 translated nucleotide sequence
ATGGAACCTTCAAAAATGGAAGTGAAAATAATATCGTCCGAAACCATCAAACCGTCATCTCCGACACCATCCCACCTTCG
AAAATATACACTTTCTTTGCTCGACCAAAAATACACGCCTATCGTTGTTCCGGCCATTCTATTCTATGAGCGCCCACAAG
GGGTGGCGCCATTGGATATGGACCGTCTCAGAACATGCCTCTCACAGACACTTACCGCGTTTTACCCTTTAGCCGGACGA
GCTGAATCTCGAGACGTTATAATATGTAATGACGAAGGTATCCCCTTCGTTGAGGCTCATGTCGATTGTGAACTTTCGAG
TGTTGTTAAGTCGCTTTCGTCCCTAGGGAGTGATTTGCGGTCTTTTTACCCGCCTAGGGACGGTTTACTCGAGGGGGGAA
TTCAGTTTGCTATTCAGATGAATGTGTTTAGTTGTGGCGGGTTTGCGTTCGCGTGGTATTGCACGCATAACGTTACTGAC
GGGACCTCGACTGCTAACTTTTTTAGGTATTGGACTGCGCTGTATGCTCAACGTAGTGAGTACGCAGTCCAAGACCTAAT
GGATTTCAATTCCGTCGTCACTGCCTTTCCCCCTGTGCCGCCCCGTGTACCGCAGGAGGAAAAACCGGTGACAACGGAAT
TGAAACCCGAGAAACAAGAGGGACAAGAAAAGGAGGAAAAGAAAAAATCGTCATTTAATTTCAGTTTTCAATCTCACATC
GTGGCGAGGAGTTTCTTGATAAAGAGCAAGGCGGTCGCAGAGTTGAAGGCCAAGTCGGTAAGCGAGGAAGTGCCATATCC
GAGTCGGTTCGAGGCCGTGTCGGCTTTCCTATGGAAATCGATAGTGTCAAGCTCGACAACAGAAGGGAAGACGATGATCA
ATATGCCCGTAAACTTGAGACCACGGGTGGACCCGCCATTACCCTTGGACTCCGTAGGTAACATTTTCGAAAATGCACTC
GTACAGTCCGAGAAAAAAGCGGAGCTCCACGAATTCGTTGCAAGGATCCGTGGATCAATCTCGAAAATGAAAGATTTTGC
CACGGAATATCAAGGCGAAAAGCGGGAAGAAGCTAAGGACGCACATTGGAAAAGATTCATAAAAGCGGTTATCGAGTGTA
AGGGGAAAGACGCCTACGTAATTTCGCCTTGGTATAAGTCGTCCGGGTTTACGGACATAGATTTCGGGTTTGGGACCCCG
ATACGGGTCGTACCCATGGACGATGTCGTAAATCATAATCAAAGGAACACGATAATGTTGATGGAGTTTGTTGATTCCGA
CGGTGATGGATTTGAAGCTTGGATGTTCCTGGAGGAGGAATGTATCAAGTTTTTGGAGTCCAACCCGGAATTTCTTGCCT
TTGCTTCCCCAAACTTTTAA
SEQ ID NO: 35 SoBAHD1 coding sequence
MEPSKMEVKIISSETIKPSSPTPSHLRKYTLSLLDQKYTPIVVPAILFYERPQGVAPLDMDRLRTCLSQTLTAFYPLAGR
AESRDVIICNDEGIPFVEAHVDCELSSVVKSLSSLGSDLRSFYPPRDGLLEGGIQFAIQMNVFSCGGFAFAWYCTHNVTD
GTSTANFFRYWTALYAQRSEYAVQDLMDFNSVVTAFPPVPPRVPQEEKPVTTELKPEKQEGQEKEEKKKSSFNFSFQSHI
VARSFLIKSKAVAELKAKSVSEEVPYPSRFEAVSAFLWKSIVSSSTTEGKTMINMPVNLRPRVDPPLPLDSVGNIFENAL
VQSEKKAELHEFVARIRGSISKMKDFATEYQGEKREEAKDAHWKRFIKAVIECKGKDAYVISPWYKSSGFTDIDFGFGTP
IRVVPMDDVVNHNQRNTIMLMEFVDSDGDGFEAWMFLEEECIKFLESNPEFLAFASPNF
SEQ ID NO: 36 SoBAHD1 translated nucleotide sequence

### Tables

TABLE 1
List of literature oxidosqualene cyclase
sequences used in phylogenetic analyses.

| Name | Accession/GenBank ID | Species |
|---|---|---|
| AtBAS | At1g78950 | Arabidopsis thaliana |
| AaBAS | EU330197 | Artemisia annua |
| AsOXA1 | AY836006 | Aster sedifolius |
| AsbAS1 | AJ311789 | Avena strigosa |
| MtbAS1 | AJ430607 | Medicago truncatula |
| PgOSCPNY1 | AB009030 | Panax ginseng |
| PsOSCPSY | AB034802 | Pisum sativum |
| SITTS1 | HQ266579 | Solanum lycopersicum |
| VhBS | DQ915167 | Vaccaria hispanica |
| AtCAS1 | At2g07050 | Arabidopsis thaliana |
| AsCS1 | AJ311790 | Avena strigosa |
| LjOSC5 | AB181246 | Lotus japonicus |
| PgOSCPNX1 | AB009029 | Panax ginseng |
| PsCASPEA | D89619 | Pisum sativum |
| LjOSC7 | AB244671 | Lotus japonicus |
| GgLUS1 | AB116228 | Glycyrrhiza glabra |
| KdLUS | HM623871 | Kalanchoe daigremontiana |
| LjOSC3 | AB181245 | Lotus japonicus |

TABLE 2
Primer oligonucleotide sequences.

| Primer Name | Sequence (5′-3′) |
|---|---|
| bAS | |
| FWD-SobAS-attB | GGGGACAAGTTTGTACAAAAAAGCAGGCTTAATGTGGAGGTTAAAAATAGCAGAAG |
| REV-SobAS-attB | GGGGACCACTTTGTACAAGAAAGCTGGGTATTAACTAAGCCTCAAAGGAACATG |
| CYP450 | |
| FWD-SoC28-attb | GGGGACAAGTTTGTACAAAAAAGCAGGCTTAATGGAACTCTTCTTCATATGTGGA |
| REV-SoC28-attb | GGGGACCACTTTGTACAAGAAAGCTGGGTATTAAGCAGATACAGTTACGGGTTT |
| FWD-SoC2816-attb | GGGGACAAGTTTGTACAAAAAAGCAGGCTTAATGGAGCTAATTACCTTACTAAGTG |
| REV-SoC2816-attb | GGGGACCACTTTGTACAAGAAAGCTGGGTATTAAGCGAGGGTGGCGGATT |
| FWD-SoC23-attb | GGGGACAAGTTTGTACAAAAAAGCAGGCTTAATGCATAGTAATAGTAAGATGGGTA |
| REV-SoC23-attb | GGGGACCACTTTGTACAAGAAAGCTGGGTATTAACGTCTACGAAACATGAGAG |
| CSL | |
| FWD-SoCSL | GGGGACAAGTTTGTACAAAAAAGCAGGCTTAATGTCACCCCACAACACCTG |
| REV-SoCSL | GGGGACCACTTTGTACAAGAAAGCTGGGTATTAAGAGCGACCTTTTCTAGCTTT |
| UGT | |
| FWD-SoC3Gal | GGGGACAAGTTTGTACAAAAAAGCAGGCTTAATGGGTTCAAATACAGAAGCAACT |
| REV-SoC3Gal | GGGGACCACTTTGTACAAGAAAGCTGGGTATCAAGCCTTCCTTAACGATCTC |
| FWD-SoC3Xyl | GGGGACAAGTTTGTACAAAAAAGCAGGCTTAATGAAGTCACCACTAAAGTTGTAC |
| REV-SoC3Xyl | GGGGACCACTTTGTACAAGAAAGCTGGGTACTAATTAGCAACCTTACTCATTTTATC |
| FWD-SoC3Fu | GGGGACAAGTTTGTACAAAAAAGCAGGCTTAATGTCGGATCAAAATGATAAAAAGGT |
| REV-SoC3Fu | GGGGACCACTTTGTACAAGAAAGCTGGGTATTAGAAAGATGAAACCCACTCAATAA |
| FWD-SoC3Rha | GGGGACAAGTTTGTACAAAAAAGCAGGCTTAATGTCTGCCAAAATGTTGCACG |
| REV-SoC3Rha | GGGGACCACTTTGTACAAGAAAGCTGGGTATCACTCGACGAGTGCTTGTAAA |
| FWD-SoC3Xyl1 | GGGGACAAGTTTGTACAAAAAAGCAGGCTTAATGGGTACTAAAGAGTTACACATAG |
| REV-SoC3Xyl1 | GGGGACCACTTTGTACAAGAAAGCTGGGTACTACTTCTCAACAAGATCTTGTAG |
| FWD-SoC3Xyl2 | GGGGACAAGTTTGTACAAAAAAGCAGGCTTAATGGAGGAATCAAAGGAGGAAG |
| REV-SoC3Xyl2 | GGGGACCACTTTGTACAAGAAAGCTGGGTATCAAAATTTTTGTAGCACAGCTTTG |
| FWD-SoBAHD1 | GGGGACAAGTTTGTACAAAAAAGCAGGCTTAATGGAAGTGAAAATTGTACGTAGG |
| REV-SoBAHD1 | GGGGACCACTTTGTACAAGAAAGCTGGGTATTAGCTGGGCGTGGCATATTC |
| Sequencing | |
| FWD-attL 1 | TCGCGTTAACGCTAGCATGGATCTC |
| REV-attL2 | ACATCAGAGATTTTGAGACACGGGC |

TABLE 3
Correlation analysis of candidate CYP450s and characterized SobAS gene
expression pattern using Pearson correlation coefficient (PCC).

| Contig | Human Readable Description | PCC |
|---|---|---|
| TRINITY_DN1084_c0_g4 (SobAS) | Terpene cyclase/mutase family member | 1.00 |
| TRINITY_DN645_c1_g2 (SoC23) | Cytochrome P450 | 0.99 |
| TRINITY_DN651_c0_g3 (SoC28) | Cytochrome P450 | 0.97 |
| TRINITY_DN5729_c1_g1 | Cytochrome P450 | 0.97 |
| TRINITY_DN2993_c0_g1 | Cytochrome P450 | 0.95 |
| TRINITY_DN13626_c1_g2 (SoC28C16) | Cytochrome P450 | 0.95 |
| TRINITY_DN58802_c0_g3 | Cytochrome P450 family protein | 0.93 |
| TRINITY_DN5664_c0_g3 | Cytochrome P450 | 0.92 |
| TRINITY_DN283414_c0_g1 | Cytochrome p450 | 0.92 |
| TRINITY_DN8790_c0_g3 | Cytochrome P450 | 0.91 |
| TRINITY_DN5664_c0_g1 | Cytochrome P450 | 0.90 |
| TRINITY_DN44858_c0_g1 | Cytochrome P450, putative | 0.89 |
| TRINITY_DN10048_c0_g1 | Cytochrome P450, putative | 0.88 |
| TRINITY_DN55859_c0_g1 | Cytochrome P450, putative | 0.88 |
| TRINITY_DN5555_c0_g1 | Cytochrome P450, putative | 0.87 |
| TRINITY_DN41487_c0_g1 | Cytochrome P450 | 0.86 |
| TRINITY_DN183736_c0_g1 | Cytochrome P450 | 0.86 |
| TRINITY_DN8560_c0_g1 | Cytochrome P450, putative | 0.86 |
| TRINITY_DN135458_c0_g1 | Cytochrome P450, putative | 0.85 |
| TRINITY_DN2210_c0_g1 | Cytochrome P450 | 0.84 |
| TRINITY_DN101327_c0_g6 | Cytochrome p450 | 0.82 |
| TRINITY_DN7831_c0_g3 | Cytochrome P450 | 0.81 |
| TRINITY_DN43050_c0_g1 | Cytochrome P450 | 0.81 |
| TRINITY_DN71147_c0_g2 | Cytochrome P450 4g15 | 0.80 |
| TRINITY_DN78115_c0_g1 | Cytochrome P450 | 0.80 |
| TRINITY_DN4811_c1_g2 | Cytochrome P450 | 0.80 |

TABLE 4
Correlation analysis of candidate UGTs and characterized SobAS gene
expression pattern using Pearson correlation coefficient (PCC).

| Contig | Human Readable Description | PCC |
|---|---|---|
| TRINITY_DN1084_c0_g4 (SobAS) | Terpene cyclase/mutase family member | 1.00 |
| TRINITY_DN1618_c1_g2 | Glycosyltransferase | 0.99 |
| TRINITY_DN28657_c0_g1 (SoC28Xyl2) | Glycosyltransferase | 0.98 |
| TRINITY_DN5570_c0_g3 | Glycosyltransferase | 0.98 |
| TRINITY_DN5701_c1_g1 (SoC28Rha) | Glycosyltransferase | 0.98 |
| TRINITY_DN3554_c0_g2 | O-fucosyltransferase | 0.97 |
| TRINITY_DN54808_c0_g7 | Glycosyltransferase | 0.97 |
| TRINITY_DN5570_c0_g1 | Glycosyltransferase | 0.96 |
| TRINITY_DN51550_c0_g1 (SoC3Gal) | Glycosyltransferase | 0.96 |
| TRINITY_DN347728_c0_g1 | Glycosyltransferase | 0.96 |
| TRINITY_DN41181_c0_g1 | Glycosyltransferase | 0.95 |
| TRINITY_DN342_c0_g1 (SoC28Fu) | Glycosyltransferase | 0.95 |
| TRINITY_DN5422_c7_g1 | UDP-glycosyltransferase | 0.95 |
| TRINITY_DN14107_c4_g1 (SoC3Xyl) | Glycosyltransferase | 0.94 |
| TRINITY_DN31287_c0_g2 | Glycosyltransferase | 0.91 |
| TRINITY_DN15200_c0_g1 | Unknown protein | 0.91 |
| TRINITY_DN586_c1_g1 (SoC28Xyl1) | Glycosyltransferase | 0.91 |

TABLE 5
Correlation analysis of candidate CSLs and characterized SobAS gene
expression pattern using Pearson correlation coefficient (PCC).

| Contig | Human Readable Description | PCC |
|---|---|---|
| TRINITY_DN1084_c0_g4 | Terpene cyclase/mutase family member | 1.00 |
| TRINITY_DN345366_c0_g1 | Cellulose synthase | 0.97 |
| TRINITY_DN23622_c0_g2 (SoCSL) | Cellulose synthase | 0.91 |
| TRINITY_DN46549_c0_g1 | Cellulose synthase | 0.90 |
| TRINITY_DN11658_c0_g2 | Cellulose synthase | 0.89 |
| TRINITY_DN57970_c0_g1 | Cellulose synthase | 0.88 |
| TRINITY_DN86505_c0_g1 | Cellulose synthase | 0.86 |
| TRINITY_DN19883_c0_g5 | Cellulose synthase | 0.85 |

TABLE 6
Amino acid sequence similarity between genes involved
in saponarioside biosynthesis in S. officinalis and
QS-21 biosynthetic genes in Q. saponaria.

| S. officinalis | Q. saponaria | AA Identity (%) |
|---|---|---|
| SobAS | QsbAS | 79.7% |
| SoC28 | QsCYP716-C28 | 74.8% |
| SoC28C16 | QsCYP716-C16 (short) | 49.0% |
| SoC23 | QsCYP714-C-23 | 33.0% |
| SoCSL | QsCslG2 | 56.0% |
| SoC3Gal | Qs-3-O-GalT | 46.3% |
| SoC3Xyl | Qs-3-O-XylT (Qs_0283870) | 47.2% |
| SoC28Fu | Qs-28-O-FucT | 43.0% |
| SoFuSyn | QsFucSyn | 57.2% |
| SoC28Rha | Qs-28-O-RhaT | 29.2% |
| SoC28Xyl1 | Qs-28-O-XylT3 | 31.1% |
| SoC28Xyl2 | Qs-28-O-XylT4 | 41.2% |

### REFERENCES

[1] Jia, Z., Koike, K. and Nikaido, T. (1998). Major triterpenoid saponins from Saponaria officinalis. Journal of Natural Products. 61: 1368-1373.

[2] Eastman, J. (2014). Wildflowers of the Eastern United States: An Introduction to Common Species of Woods, Wetlands and Fields. Stackpole Books.

[3] Rees, A. (1819). The cyclopædia; or, universal dictionary of arts, sciences, and literature (Vol. 4). Longman, Hurst, Rees, Orme and Brown.

[4] Korkmaz, M. and Özçelik, H. (2011). Economic importance of Gypsophila L., Ankyropetalum fenzl and Saponaria L. (Caryophyllaceae) taxa of Turkey. African journal of Biotechnology, 10(47), 9533-9541.

[5] Böttger, S. and Melzig, M. F. (2011). Triterpenoid saponins of the Caryophyllaceae and Illecebraceae family. Phytochemistry Letters. 4: 59-68.

[6] Smułek, W., Zdarta, A., Pacholak, A., Zgoła-Grześkowiak, A., Marczak, Ł., Jarzębski, M., and Kaczorek, E. (2017). Saponaria officinalis L. extract: Surface active properties and impact on environmental bacterial strains. Colloids and Surfaces B: Biointerfaces, 150, 209-215.

[7] Gonzalez, P. J. and Sörensen, P. M. (2020). Characterization of saponin foam from Saponaria officinalis for food applications. Food Hydrocolloids, 101, 105541.

[8] Sadowska, B., Budzyńska, A., Więckowska-Szakiel, M., Paszkiewicz, M., Stochmal, A., Moniuszko-Szajwaj, B., Kowalczyk, M. and Różalska, B. (2014). New pharmacological properties of Medicago sativa and Saponaria officinalis saponin-rich fractions addressed to Candida albicans. Journal of medical microbiology, 63(8), 1076-1086.

[9] Gilabert-Oriol, R., Thakur, M., Haussmann, K., Niesler, N., Bhargava, C., Görick, C., Fuchs, H. and Weng, A. (2016). Saponins from Saponaria officinalis L. augment the efficacy of a rituximab-immunotoxin. Planta medica, 82(18), 1525-1531.

[10] Reed, J., Orme, A., El-Demerdash, A., Owen, C., Martin, L. B., Misra, R. C., . . . & Osbourn, A. (2023). Elucidation of the pathway for biosynthesis of saponin adjuvants from the soapbark tree. Science, 379(6638), 1252-1264.

1. A method for the production of a triterpenoid comprising;
(i) contacting OS with a Saponaria officinalis β-amyrin synthase (SobAS) comprising an amino acid sequence having at least 80% sequence identity to SEQ ID NO 8, such that said OS is converted into β-amyrin;
(ii) either;
a) contacting β-amyrin with a SoC28 oxidase polypeptide comprising an amino acid sequence having at least 80% sequence identity to SEQ ID NO 2, such that the C28 position of said β-amyrin is oxidised to a carboxylic acid to produce oleanolic acid; and contacting oleanolic acid with a SoC28C16 oxidase polypeptide comprising an amino acid sequence having at least 50% sequence identity to SEQ ID NO 4, such that the C16 position of said oleanolic acid is oxidised to an alcohol, thereby producing echinocystic acid, or
b) contacting oleanolic acid with a SoC28C16 oxidase polypeptide comprising an amino acid sequence having at least 50% sequence identity to SEQ ID NO 4, such that the C16 position of said oleanolic acid is oxidised to an alcohol and the C28 position of said β-amyrin is oxidised to a carboxylic acid, thereby producing echinocystic acid;
(iii) contacting echinocystic acid with a SoC23 oxidase polypeptide comprising an amino acid sequence having at least 50% sequence identity to SEQ ID NO 6, such that the C-23 position of said echinocystic acid is oxidised to an aldehyde, thereby producing quillaic acid (QA);
(iv) contacting QA with Saponaria officinalis QA 3-O glucuronosyl transferase (“SoCSL”) polypeptide comprising an amino acid sequence having at least 60% sequence identity to SEQ ID NO: 10, such that said QA is converted into QA-GlcA;
(v) contacting QA-GlcA with a Saponaria officinalis QA-GlcA galactosyl transferase (“SoC3Gal”) polypeptide comprising an amino acid sequence having at least 50% sequence identity to SEQ ID NO: 12, such that said QA-GlcA is converted into QA-GlcA-Gal;
(vi) contacting QA-GlcA with a Saponaria officinalis QA-GlcA-Gal xylosyl transferase (“SoC3Xyl”) polypeptide comprising an amino acid sequence having at least 50% sequence identity to SEQ ID NO: 14, such that said QA-GlcA-Gal is converted into QA-Tri;
(vii) contacting QA-Tri with a Saponaria officinalis QA-Tri fucosyl transferase (“SoC28Fu”) polypeptide comprising an amino acid sequence having at least 60% sequence identity to SEQ ID NO: 16, such that said QA-Tri is converted into QA-TriF;
(viii) contacting QA-TriF with a Saponaria officinalis QA-TriF rhamnosyl transferase (“SoC28Rha”) polypeptide comprising an amino acid sequence having at least 50% sequence identity to SEQ ID NO: 18, such that said QA-TriF is converted into QA-TriFR;
(ix) contacting QA-TriFR with a Saponaria officinalis QA-TriFR xylosyl transferase (“SoC28Xyl1”) polypeptide comprising an amino acid sequence having at least 50% sequence identity to SEQ ID NO: 20, such that said QA-TriFR is converted into QA-TriFRX;
(x) contacting QA-TriFRX with a Saponaria officinalis QA-TriFRX xylosyl transferase (“SoC28Xyl2”) polypeptide comprising an amino acid sequence having at least 50% sequence identity to SEQ ID NO: 22, such that said QA-TriFRX is converted into QA-TriFRXX,
(xi) contacting QA-TriFRXX with a Saponaria officinalis QA-TriFRXX quinovosyl transferase (“SoGH1”) polypeptide comprising an amino acid sequence having at least 50% sequence identity to SEQ ID NO: 34, such that said QA-TriFRXX is converted into QA-TriF(Q)RXX; and/or
(xii) contacting QA-TriF(Q)RXX with a Saponaria officinalis QA-TriF(Q)RXX acetyl transferase (“SoBAHD1”) polypeptide comprising an amino acid sequence having at least 50% sequence identity to SEQ ID NO: 36, such that said QA-TriF(Q)RXX is converted into saponarioside B (SpB).

2. A method according to claim 1 comprising;
(i) either
(a) contacting β-amyrin with a Saponaria officinalis C28 oxidase (SoC28 oxidase) to oxidise the C28 position of the β-amyrin to a carboxylic acid to form oleanolic acid, wherein the amino acid sequence of the SoC28 oxidase has at least 80% sequence identity to SEQ ID NO: 2; and
contacting oleanolic acid with a Saponaria officinalis C28C16 oxidase (SoC28C16 oxidase) to oxidise the C16 position of the oleanolic acid to an alcohol to form echinocystic acid, wherein the amino acid sequence of the C16 oxidase has at least 50% sequence identity to SEQ ID NO: 4; or
(b) contacting β-amyrin with a Saponaria officinalis C28C16 oxidase (SoC28C16 oxidase) to oxidise the C28 position of the β-amyrin to a carboxylic acid and the C16 position to an alcohol to form echinocystic acid, wherein the amino acid sequence of the C28C16 oxidase has at least 50% sequence identity to SEQ ID NO: 4
(iii) contacting echinocystic acid with a Saponaria officinalis C-23 oxidase (SoC23 oxidase) to oxidise the C-23 position of echinocystic acid to an aldehyde to form quillaic acid (QA), wherein the amino acid sequence of the SoC23 oxidase having at least 50% sequence identity to SEQ ID NO: 6.

3. A method according to claim 2 wherein β-amyrin is produced by contacting 2,3-oxidosqualene (OS) with a β-amyrin synthase (SobAS) having an amino acid sequence with at least 80% sequence identity to SEQ ID NO: 8; thereby cyclising the OS to produce β-amyrin.

4. A method according to claim 2 or claim 3 further comprising;
(iv) contacting QA with a Saponaria officinalis QA 3-O glucuronosyl transferase (“SoCSL”) to covalent attach D-glucuronic acid (“GlcA”) to the 3-O position of quillaic acid to form 3-O-{β-D-glucopyranosiduronic acid}-quillaic acid (“QA-GlcA”); wherein the amino acid sequence of the SoCSL having at least 60% sequence identity to SEQ ID NO: 10;
(v) contacting QA-GlcA with Saponaria officinalis QA-GlcA galactosyl transferase (“SoC3Gal”) to covalently attach D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-GlcA-Gal”); wherein the amino acid sequence of the QA-GlcA-Gal has at least 50% sequence identity to SEQ ID NO: 12; and
(vi) contacting QA-GlcA-Gal with a Saponaria officinalis QA-GlcA-Gal xylosyl transferase (“SoC3Xyl”) to covalently attach D-Xylose (“Xyl”) via a 1,3 linkage to QA-GlcA-Gal to form 3-O-{β-D-xylopyranosyl-(1->3)-[β-D-galactopyranosyl-(1->2)]-3-D-glucopyranosiduronic acid}-quillaic acid (“QA-GlcA-[Gal]-Xyl” QA-Tri); wherein the amino acid sequence of SoC3Xyl has at least 50% sequence identity to SEQ ID NO: 14.

5. A method according to claim 4 further comprising;
(vii) contacting 3-O-{β-D-xylopyranosyl-(1->3)-[3-D-galactopyranosyl-(1->2)]-β-D-glucopyranosiduronic acid}-quillaic acid (QA-Tri) with a Saponaria officinalis QA-Tri fucosyl transferase (“SoC28Fu”) to attach fucose to the 28-O position QA-Tri to form 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF); wherein the amino acid sequence of QATriFuT has at least 60% sequence identity to SEQ ID NO: 16;
(viii) contacting QA-TriF with a Saponaria officinalis QA-TriF rhamnosyl transferase (“SoC28Rha”) to covalently attach rhamnose via a 1, 2 linkage to QA-TriF to form 3-O-{β-D-xylopyranosyl-(1->3)-[3-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR); wherein the amino acid sequence of SoC28Rha has at least 50% sequence identity to SEQ ID NO: 18;
(ix) contacting QA-TriFR with a Saponaria officinalis QA-TriFR xylosyl transferase (“SoC28Xyl1”) to covalently attach xylose via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX); wherein the amino acid sequence of SoC28Xyl1 has at least 50% sequence identity to SEQ ID NO: 20; and
(x) contacting QA-TriFRX with a Saponaria officinalis QA-TriFRX-xylosyl transferase (“SoC28Xyl2”) to covalently attach xylose via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-3-D-fucopyranosyl ester}-quillaic acid (QA-TriFRXX); wherein the amino acid sequence of SoC28Xyl2 has at least 50% sequence identity to SEQ ID NO: 22.

6. A method according to claim 5 further comprising
contacting QA-TriFRXX with a Saponaria officinalis QA-TriFRXX quinovosyl transferase (“SoGH1”) to covalently attach quinovose via a 1,4 linkage to QA-TriFRXX to form 3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[β-D-quinovopyranosyl-(1→4)]-3-D-fucopyranosyl ester}-quillaic acid (QA-TriF(Q)RXX), wherein the amino acid sequence of SoGH1 has at least 50% sequence identity to SEQ ID NO: 34; and
contacting QA-TriF(Q)RXX with a Saponaria officinalis QA-TriF(Q)RXX acetyl transferase (“SoBAHD1”) to covalently attach an acetyl group to QA-TriF(Q)RXX to form QA-TriF(Q-Ac)RXX (saponarioside B), wherein the amino acid sequence of SoBAHD1 has at least 50% sequence identity to SEQ ID NO: 36.

7. A method of converting a host from a phenotype whereby the host is unable to carry out triterpenoid biosynthesis from 2,3-oxidosqualene (OS) to a phenotype whereby the host is able to carry out said triterpenoid biosynthesis, the method comprising;
expressing a heterologous nucleic acid within the host or one or more cells thereof, following an earlier step of introducing the nucleic acid into the host or an ancestor of either,
wherein the heterologous nucleic acid encodes one or more of;
(i) a SoC28 oxidase capable of oxidising β-amyrin at the C28 position to a carboxylic acid to form oleanolic acid; said SoC28 oxidase having at least 80% sequence identity to SEQ ID NO: 2;
(ii) a SoC28C16 oxidase capable of oxidising β-amyrin at the C28 position to a carboxylic acid and at the C16 position to an alcohol (“C16 oxidase”) to form echinocystic acid; said SoC28C16 oxidase having at least 50% sequence identity to SEQ ID NO: 4;
(iii) a SoC23 oxidase capable of oxidising echinocystic acid at the C-23 position to an aldehyde to form quillaic acid (QA), said SoC23 oxidase having at least 50% sequence identity to SEQ ID NO: 6;
(iv) a Saponaria officinalis QA 3-O glucuronosyl transferase (“SoCSL”) for attachment of D-glucuronic acid (“GlcA”) to the 3-O position of quillaic acid to form 3-O-{β-D-glucopyranosiduronic acid}-quillaic acid (“QA-GlcA”); said SoCSL having at least 60% sequence identity to SEQ ID NO: 10;
(v) Saponaria officinalis QA-GlcA galactosyl transferase (“SoC3Gal”) for attachment D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-GlcA-Gal”); wherein the amino acid sequence of the SoC3Gal has at least 50% sequence identity to SEQ ID NO: 12; and
(vi) a Saponaria officinalis QA-GlcA-Gal xylosyl transferase (“SoC3Xyl”) for attachment of D-Xylose (“Xyl”) via a 1,3 linkage to QA-GlcA-Gal to form 3-O-{β-D-xylopyranosyl-(1->3)-[β-D-galactopyranosyl-(1->2)]-β-D-glucopyranosiduronic acid}-quillaic acid (“QA-GlcA-[Gal]-Xyl” QA-Tri); wherein the amino acid sequence of the SoC3Xyl has at least 50% sequence identity to SEQ ID NO: 14; and
(vii) a Saponaria officinalis QA-Tri fucosyl transferase (“SoC28Fu”) for the attachment of fucose(“Fuc”) to the 28-O position of QA-Tri to form 3-O-{β-D-xylopyranosyl-(1->3)-[3-D-galactopyranosyl-(1->2)]-β-D-glucopyranosiduronic acid}-28-O-{β-D-fucopyranosyl ester}-quillaic acid (QA-TriF); said SoC28Fu having at least 60% sequence identity to SEQ ID NO: 16;
(viii) a Saponaria officinalis QA-TriF rhamnosyl transferase (“SoC28Rha”) for the attachment of rhamnose (“Rha”) via a 1, 2 linkage to QA-TriF to form 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR); said SoC28Rha having at least 50% sequence identity to SEQ ID NO: 18;
(ix) Saponaria officinalis QA-TriFR xylosyl transferase (“SoC28Xyl1”) for attachment of D-Xylose (“Xyl”) via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX)”); wherein the amino acid sequence of the SoC28Xyl1 has at least 50% sequence identity to SEQ ID NO: 20;
(x) a Saponaria officinalis QA-TriFRX xylosyl transferase (“SoC28Xyl2”) for attachment of D-Xylose (“Xyl”) via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX); wherein the amino acid sequence of SoC28Xyl2has at least 80% sequence identity to SEQ ID NO:22,
(xi) a Saponaria officinalis QA-TriFRXX quinovosyl transferase (“SoGH1”) for attachment of quinovose via a 1, 4 linkage to QA-TrFRXX to form 3-O-{β-D-xylopyranosyl-(1→3)-[3-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[3-D-quinovopyranosyl-(1→4)]-3-D-fucopyranosyl ester}-quillaic acid (QA-TriF(Q)RXX), wherein the amino acid sequence of SoGH1 has at least 50% sequence identity to SEQ ID NO: 34, and/or
(xii) a Saponaria officinalis QA-TriF(Q)RXX acetyl transferase (“SoBAHD1”) for attachment of an acetyl group to QA-TriF(Q)RXX to form saponarioside B, wherein the amino acid sequence of SoBAHD1 has at least 50% sequence identity to SEQ ID NO: 36.

8. A method according to claim 7 wherein the heterologous nucleic acid encodes the following polypeptides;
(i) a SoC28 oxidase capable of oxidising β-amyrin thereof at the C28 position to a carboxylic acid to form oleanolic acid; said SoC28 oxidase having at least 80% sequence identity to SEQ ID NO: 2;
(ii) a SoC28C16 oxidase capable of oxidising β-amyrin at the C28 position to a carboxylic acid and at the C16 position to an alcohol (“C16 oxidase”) to form echinocystic acid; said SoC28C16 oxidase having at least 50% sequence identity to SEQ ID NO: 4; and
(iii) a SoC23 oxidase capable of oxidising echinocystic acid at the C-23 position to an aldehyde to form quillaic acid (QA), said SoC23 oxidase having at least 50% sequence identity to SEQ ID NO: 6.

9. A method according to claim 8 wherein the heterologous nucleic acid further encodes a Saponaria officinalis β-amyrin synthase (SobAS) for cyclisation of OS to a triterpene; said SobAS having at least 80% sequence identity to SEQ ID NO: 8.

10. A method according to claim 8 or claim 9 wherein the heterologous nucleic acid further encodes the following polypeptides;
(iv) a Saponaria officinalis QA 3-O glucuronosyl transferase (“SoCSL”) for attachment of D-glucuronic acid (“GlcA”) to the 3-O position of quillaic acid to form 3-O-{β-D-glucopyranosiduronic acid}-quillaic acid (“QA-GlcA”); said SoCSL having at least 60% sequence identity to SEQ ID NO: 10;
(v) Saponaria officinalis QA-GlcA galactosyl transferase (“SoC3Gal”) for attachment D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-GlcA-Gal”); wherein the amino acid sequence of the SoC3Gal has at least 50% sequence identity to SEQ ID NO: 12; and
(vi) a Saponaria officinalis QA-GlcA-Gal xylosyl transferase (“SoC3Xyl”) for attachment of D-Xylose (“Xyl”) via a 1,3 linkage to QA-GlcA-Gal to form 3-O-{β-D-xylopyranosyl-(1->3)-[β-D-galactopyranosyl-(1->2)]-β-D-glucopyranosiduronic acid}-quillaic acid (“QA-GlcA-Gal-Xyl” or “QA-Tri”); wherein the amino acid sequence of SoC3Xyl has at least 50% sequence identity to SEQ ID NO: 14.

11. A method according to claim 10 wherein the heterologous nucleic acid further encodes the following polypeptides;
(vii) a Saponaria officinalis QA-Tri fucosyl transferase (“SoC28Fu”) for the attachment of fucose(“Fuc”) to the 28-O position of QA-Tri to form 3-O-{β-D-xylopyranosyl-(1->3)-[β-D-galactopyranosyl-(1->2)]-3-D-glucopyranosiduronic acid}-28-O-{β-D-fucopyranosyl ester}-quillaic acid (QA-TriF); said SoC28Fu having at least 60% sequence identity to SEQ ID NO: 16;
(viii) a Saponaria officinalis QA-TriF rhamnosyl transferase (“SoC28Rha”) for the attachment of rhamnose (“Rha”) via a 1, 2 linkage to QA-TriF to form 3-O-{β-D-xylopyranosyl-(1->3)-[3-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR); said SoC28Rha having at least 50% sequence identity to SEQ ID NO: 18;
(ix) Saponaria officinalis QA-TriFR xylosyl transferase (“SoC28Xyl1”) for attachment of D-Xylose (“Xyl”) via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX); wherein the amino acid sequence of the SoC28Xyl1 has at least 50% sequence identity to SEQ ID NO: 20; and
(x) a Saponaria officinalis QA-TriFRX xylosyl transferase (“SoC28Xyl2”) for attachment of D-Xylose (“Xyl”) via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX); wherein the amino acid sequence of SoC28Xyl2 has at least 80% sequence identity to SEQ ID NO:22.

12. A method according to claim 11 wherein the heterologous nucleic acid further encodes the following polypeptides;
(xi) a Saponaria officinalis QA-TriFRXX quinovosyl transferase (“SoGH1”) for attachment of quinovose (Q) via a 1, 4 linkage to QA-TrFRXX to form 3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[3-D-quinovopyranosyl-(1→4)]-β-D-fucopyranosyl ester}-quillaic acid (QA-TriF(Q)RXX), wherein the amino acid sequence of SoGH1 has at least 50% sequence identity to SEQ ID NO: 34, and
(xii) a Saponaria officinalis QA-TriF(Q)RXX acetyl transferase (“SoBAHD1”) for attachment of an acetyl group to QA-TriF(Q)RXX to form saponarioside B, wherein the amino acid sequence of SoBAHD1 has at least 50% sequence identity to SEQ ID NO: 36.

13. A host cell containing or transformed with a heterologous nucleic acid which comprises a plurality of nucleotide sequences each of which encodes a polypeptide which in combination have triterpenoid biosynthesis activity,
wherein the plurality of nucleotide sequences encode one or more of the following polypeptides;
(i) a Saponaria officinalis β-amyrin synthase (SobAS) for cyclisation of OS to a triterpene; said SobAS having at least 80% sequence identity to SEQ ID NO: 8.
(ii) a SoC28 oxidase capable of oxidising β-amyrin thereof at the C28 position to a carboxylic acid to form oleanolic acid; said SoC28 oxidase having at least 80% sequence identity to SEQ ID NO: 2;
(iii) a SoC28C16 oxidase capable of oxidising β-amyrin at the C28 position to a carboxylic acid and at the C16 position to an alcohol (“C16 oxidase”) to form echinocystic acid; said SoC28C16 oxidase having at least 50% sequence identity to SEQ ID NO: 4; and
(iv) a SoC23 oxidase capable of oxidising echinocystic acid at the C-23 position to an aldehyde to form quillaic acid (QA), said SoC23 oxidase having at least 50% sequence identity to SEQ ID NO: 6;
(v) a Saponaria officinalis QA 3-O glucuronosyl transferase (“SoCSL”) for attachment of D-glucuronic acid (“GlcA”) to the 3-O position of quillaic acid to form 3-O-{β-D-glucopyranosiduronic acid}-quillaic acid (“QA-GlcA”); said SoQA-GlcT having at least 60% sequence identity to SEQ ID NO: 10;
(vi) Saponaria officinalis QA-GlcA galactosyl transferase (“SoC3Gal”) for attachment D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-GlcA-Gal”); wherein the amino acid sequence of the QA-GlcA-Gal has at least 50% sequence identity to SEQ ID NO: 12;
(vii) a Saponaria officinalis QA-GlcA-Gal xylosyl transferase (“SoC3Xyl”) for attachment of D-Xylose (“Xyl”) via a 1,3 linkage to QA-GlcA-Gal to form 3-O-{β-D-xylopyranosyl-(1->3)-[β-D-galactopyranosyl-(1->2)]-3-D-glucopyranosiduronic acid}-quillaic acid (“QA-GlcA-[Gal]-Xyl” QA-Tri), wherein the amino acid sequence of SoC3Xyl has at least 50% sequence identity to SEQ ID NO: 14;
(viii) a Saponaria officinalis QA-Tri fucosyl transferase (“SoC28Fu”) for the attachment of fucose(“Fuc”) to the 28-O position of QA-Tri to form 3-O-{β-D-xylopyranosyl-(1->3)-[3-D-galactopyranosyl-(1->2)]-β-D-glucopyranosiduronic acid}-28-O-{β-D-fucopyranosyl ester}-quillaic acid (QA-TriF); said SoC28Fu having at least 60% sequence identity to SEQ ID NO: 16;
(ix) a Saponaria officinalis QA-TriF rhamnosyl transferase (“SoC28Rha”) for the attachment of rhamnose (“Rha”) via a 1, 2 linkage to QA-TriF to form 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR); said SoC28Rha having at least 50% sequence identity to SEQ ID NO: 18;
(x) Saponaria officinalis QA-TriFR xylosyl transferase (“SoC28Xyl1”) for attachment of D-Xylose (“Xyl”) via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX); wherein the amino acid sequence of the SoC28Xyl1 has at least 50% sequence identity to SEQ ID NO: 20; and/or
(xi) a Saponaria officinalis QA-TriFRX xylosyl transferase (“SoC28Xyl2”) for attachment of D-Xylose (“Xyl”) via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX); wherein the amino acid sequence of SoC28Xyl2has at least 50% sequence identity to SEQ ID NO:22.
(xii) a Saponaria officinalis QA-TriFRXX quinovosyl transferase (“SoGH1”) for attachment of quinovose (Q) via a 1, 4 linkage to QA-TriFRXX to form 3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[β-D-quinovopyranosyl-(1→4)]-β-D-fucopyranosyl ester}-quillaic acid (QA-TriF(Q)RXX), wherein the amino acid sequence of SoGH1 has at least 50% sequence identity to SEQ ID NO: 34, and/or
(xiii) a Saponaria officinalis QA-TriF(Q)RXX acetyl transferase (“SoBAHD1”) for attachment of an acetyl group to QA-TriF(Q)RXX to form saponarioside B, wherein the amino acid sequence of SoBAHD1 has at least 50% sequence identity to SEQ ID NO: 36.
wherein expression of said nucleic acid imparts on the transformed host the ability to carry out triterpenoid biosynthesis.

14. A host cell according to claim 13 wherein the plurality of nucleotide sequences encode the following polypeptides;
(i) a SoC28 oxidase capable of oxidising β-amyrin thereof at the C28 position to a carboxylic acid to form oleanolic acid; said SoC28 oxidase having at least 80% sequence identity to SEQ ID NO: 2;
(ii) a SoC28C16 oxidase capable of oxidising β-amyrin at the C28 position to a carboxylic acid and at the C16 position to an alcohol (“C16 oxidase”) to form echinocystic acid; said SoC28C16 oxidase having at least 50% sequence identity to SEQ ID NO: 4; and
(iii) a SoC23 oxidase capable of oxidising echinocystic acid at the C-23 position to an aldehyde to form quillaic acid (QA), said SoC23 oxidase having at least 50% sequence identity to SEQ ID NO: 6.
wherein expression of said nucleic acid imparts on the transformed host the ability to carry out QA biosynthesis.

15. A host cell according to claim 14 wherein the heterologous nucleic acid further encodes a Saponaria officinalis β-amyrin synthase (SobAS) for cyclisation of OS to a triterpene; said SobAS having at least 80% sequence identity to SEQ ID NO: 8.

16. A host cell according to claim 14 or claim 15 wherein the heterologous nucleic acid further encodes the following polypeptides
(iv) a Saponaria officinalis QA 3-O glucuronosyl transferase (“SoCSL”) for attachment of D-glucuronic acid (“GlcA”) to the 3-O position of quillaic acid to form 3-O-{β-D-glucopyranosiduronic acid}-quillaic acid (“QA-GlcA”); said SoCSL having at least 60% sequence identity to SEQ ID NO: 10;
(v) Saponaria officinalis QA-GlcA galactosyl transferase (“SoC3Gal”) for attachment D-Galactose (“Gal”) via a β-1->2 linkage to QA-GlcA to form 3-O-{[β-D-galactopyranosyl-(1->2)]-β-D-glucopyranosiduronic acid}-quillaic acid (“QA-GlcA-Gal”); wherein the amino acid sequence of the SoC3Gal has at least 50% sequence identity to SEQ ID NO: 12; and
(vi) a Saponaria officinalis QA-GlcA-Gal xylosyl transferase (“SoC3Xyl”) for attachment of D-Xylose (“Xyl”) via a 1,3 linkage to QA-GlcA-Gal to form 3-O-{β-D-xylopyranosyl-(1->3)-[β-D-galactopyranosyl-(1->2)]-β-D-glucopyranosiduronic acid}-quillaic acid (“QA-GlcA-Gal-Xyl” QA-Tri), wherein the amino acid sequence of SoC3Xyl has at least 50% sequence identity to SEQ ID NO: 14.

17. A host cell according to claim 16 wherein the heterologous nucleic acid further encodes one, two, three or all four of the following polypeptides;
(vii) a Saponaria officinalis QA-Tri fucosyl transferase (“SoC28Fu”) for the attachment of fucose(“Fuc”) to the 28-O position of QA-Tri to form 3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid (QA-TriF); said SoC28Fu having at least 60% sequence identity to SEQ ID NO: 16;
(viii) a Saponaria officinalis QA-TriF rhamnosyl transferase (“SoC28Rha”) for the attachment of rhamnose (“Rha”) via a 1, 2 linkage to QA-TriF to form 3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid (QA-TriFR); said SoC28Rha having at least 50% sequence identity to SEQ ID NO: 18;
(ix) Saponaria officinalis QA-TriFR xylosyl transferase (“SoC28Xyl1”) for attachment of D-Xylose (“Xyl”) via a 1,4 linkage to QA-TriFR to form 3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid (QA-TriFRX)”); wherein the amino acid sequence of the SoC28Xyl1 has at least 50% sequence identity to SEQ ID NO: 20; and
(x) a Saponaria officinalis QA-TriFRX xylosyl transferase (“SoC28Xyl2”) for attachment of D-Xylose (“Xyl”) via a 1,3 linkage to QA-TriFRX to form 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriFRXX), wherein the amino acid sequence of SoC28Xyl2has at least 50% sequence identity to SEQ ID NO:22.

18. A host cell according to claim 17 wherein the heterologous nucleic acid further encodes one or both of the following polypeptides;
(xi) a Saponaria officinalis QA-TriFRXX quinovosyl transferase (“SoGH1”) for attachment of quinovose (Q) via a 1, 4 linkage to QA-TrFRXX to form 3-O-{β-D-xylopyranosyl-(1→3)-[β-D-galactopyranosyl-(1→2)]-β-D-glucopyranosiduronic acid}-28-O-{β-D-xylopyranosyl-(1→3)-β-D-xylopyranosyl-(1→4)-α-L-rhamnopyranosyl-(1→2)-[β-D-quinovopyranosyl-(1→4)]-β-D-fucopyranosyl ester}-quillaic acid (QA-TriF(Q)RXX), wherein the amino acid sequence of SoGH1 has at least 50% sequence identity to SEQ ID NO: 34, and
(xii) a Saponaria officinalis QA-TriF(Q)RXX acetyl transferase (“SoBAHD1”) for attachment of an acetyl group to QA-TriF(Q)RXX to form saponarioside B, wherein the amino acid sequence of SoBAHD1 has at least 50% sequence identity to SEQ ID NO: 36.

19. An isolated polypeptide comprising;
(i) a SobAS amino acid sequence with at least 80% sequence identity to SEQ ID NO: 8;
(ii) a SoC28 oxidase amino acid sequence with at least 80% sequence identity to SEQ ID NO: 2
(iii) a SoC16C28 oxidase amino acid sequence with at least 50% sequence identity to SEQ ID NO: 4;
(iv) a SoC23 oxidase amino acid sequence with at least 50% sequence identity to SEQ ID NO: 6;
(v) a SoCSL amino acid sequence with at least 60% sequence identity to SEQ ID NO: 10;
(vi) a SoC3Gal amino acid sequence with at least 50% sequence identity to SEQ ID NO: 12;
(vii) a SoQA-RXylT amino acid sequence with at least 50% sequence identity to SEQ ID NO: 14;
(viii) a SoC28Fu amino acid sequence with at least 60% sequence identity to SEQ ID NO: 16;
(ix) a SoC28Rha amino acid sequence with at least 50% sequence identity to SEQ ID NO: 18;
(x) a SoC28Xyl1 amino acid sequence with at least 50% sequence identity to SEQ ID NO: 20;
(xi) a SoC28Xyl2 amino acid sequence with at least 50% sequence identity to SEQ ID NO: 22;
(xii) a SoGH1 amino acid sequence with at least 50% sequence identity to SEQ ID NO: 34 and/or (xiii) a SoBAHD1 amino acid sequence with at least 50% sequence identity to SEQ ID NO: 36.

20. An isolated nucleic acid encoding one or more polypeptides according to claim 19.

21. A vector comprising a nucleic acid according to claim 20.

22. A host cell comprising a nucleic acid according to claim 20 or a vector according to claim 21.

23. A method of producing a host cell comprising transforming or transfecting a host cell with a heterologous nucleic acid which comprises a plurality of nucleotide sequences as set out in any one of claims 7 to 18 and 20.

24. A method according to claim 23 wherein the host cell is a plant cell

25. A process for producing a transgenic plant which method comprises the steps of:
(a) performing a method of claim 24, and
(b) regenerating a plant from the transformed plant cell.

26. A transgenic plant which is obtainable by the method of claim 25, or which is a clone, or selfed or hybrid progeny or other descendant of said transgenic plant,
wherein expression of said heterologous nucleic acid imparts an increased ability to carry out the triterpenoid biosynthesis compared to a wild-type plant otherwise corresponding to said transgenic plant.

27. A method of producing a triterpenoid in a heterologous host, which method comprises culturing a host cell as set out in any one of claims 13 to 18 and 22 and purifying the triterpenoid therefrom.

28. A method of producing a triterpenoid in a heterologous host, which method comprises growing a plant according to claim 26 and then harvesting it and purifying the triterpenoid therefrom.

29. A method according to claim 27 or 28 wherein the triterpenoid is QA or glycosylated QA.

30. A method according to claim 29 wherein the glycosylated QA is QA-Tri, QA-TriFRXX or QA-TriF(Q-Ac)RXX.
